(19) United States
(12) Patent Application Publication
(10) Pub. No.: US 2026/0176249 A1
(43) Pub. Date: Jun. 25, 2026

(54) PROTEIN KINASE INHIBITOR, PREPARATION METHOD THEREFOR, AND APPLICATION THEREOF

(71) Applicant: SHANDONG LUYE PHARMACEUTICAL CO., LTD., Yantai, Shandong (CN)

(72) Inventors: Jingwei TIAN, Yantai, Shandong (CN); Liang YE, Yantai, Shandong (CN); Jing LU, Yantai, Shandong (CN); Jianzhao ZHANG, Yantai, Shandong (CN); Hui LEI, Yantai, Shandong (CN); Xinfa BAI, Yantai, Shandong (CN); Wenyan WANG, Yantai, Shandong (CN); Rui ZHANG, Yantai, Shandong (CN)

(21) Appl. No.: 18/718,412

(22) PCT Filed: Dec. 9, 2022
(86) PCT No.: PCT/CN2022/137824

§ 371 Date: Jun. 10, 2024

(30) Foreign Application Priority Data
Dec. 10, 2021 (CN) 202111509875.0
Jan. 28, 2022 (CN) 202210106980.8

(51) Int. Cl. C07D 401/12; C07D 401/04; C07D 487/04

(52) U.S. Cl. CPC C07D 401/12; C07D 401/04; C07D 487/04

(57) ABSTRACT

The present invention relates to a series of compounds for regulating activity of a protein kinase, in particular a compound for inhibiting activity of PLK1. The present invention further provides a pharmaceutical composition containing the compound, and a use of the compound and the pharmaceutical composition thereof in preparing a drug for preventing or treating diseases caused by and/or related to dysregulation of PLK1 kinase activity.

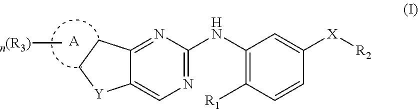

### TECHNICAL FIELD

The present invention belongs to the technical field of medicines, and relates to compounds and pharmaceutical compositions for the prevention or treatment of diseases related to dysregulation of protein kinase activity, as well as a method of using same and the use thereof. In particular, the present invention provides compounds that can serve as inhibitors of protein kinases, especially PLK1 kinase, and the use thereof.

### BACKGROUND ART

PLK1 is a serine/threonine kinase widely present in eukaryotic cells. PLK1 is involved in most processes of cell division, mainly including the entry of cell division, rupture of the nuclear membrane, centriole duplication, complete arrangement of chromosomes, initiation of mitotic checkpoints, and cytokinesis and other biological processes.

In G2 phase, PLK1 plays an important role in the activation of cyclin B-CDK1 complex through at least two mechanisms. First, it activates CDC25C phosphatase, which in turn eliminates inhibitory phosphorylation of CDK1, thereby activating CDK1. Secondly, PLK1 induces the phosphorylation-dependent degradation of WEE1, thereby preventing further phosphorylation of CDK1 and allowing cells to enter M phase. In addition, PLK1 is also involved in the regulation of DNA damage checkpoints in G2 and M phases.

Studies have confirmed that PLK1 is overexpressed in a variety of tumor tissues. Overexpression of wild-type PLK1 may lead to multinucleation, and the expression of overactive PLK1 may allow cells to bypass the DNA damage-induced G2 arrest checkpoint. In addition, PLK1 is closely related to the occurrence and development of tumors, and the expression level of PLK1 is associated with poor prognosis of clinical patients. Therefore, PLK1 inhibitors are expected to become effective anti-tumor drug targets.

Volasertib and Onvansertib are ATP-competitive PLK1 small molecule inhibitors with rapid clinical trial progress at present. Whether used as single agents or in combination, they have shown significant anti-tumor effects in a variety of preclinical models or clinical studies. Volasertib is an injectable formulation for which an increased rate of higher-grade adverse events are observed in clinical trials. Onvansertib is an oral formulation that has shown low bioavailability in preclinical studies and a narrow safety margin in clinical trials. Therefore, there is still a need in the art for compounds with high selectivity, high activity and better safety to meet clinical needs.

### SUMMARY OF THE INVENTION

In one embodiment of the present invention, there is provided a compound of formula (I), or a pharmaceutically acceptable salt or stereoisomer thereof:

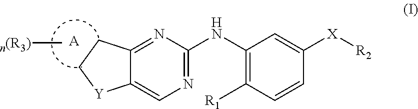

wherein:
X is independently selected from: divalent group of piperidine, piperazine, 3,8-diazabicyclo[3.2.1]octane, —CH₂-piperazine-, —NR-pyrrolidine-, —NR-azetidine-, or —NR—CH₂-pyrrolidine-; the R is independently selected from: H, D, or C₁-C₆ alkyl;
Y is independently selected from: divalent group of —C(R₄)₂—C(R₄)₂—, —CR₄═CR₄—, —C(R₄)₂—NR₄—, —NR₄—C(R₄)₂—, —CR₄═N— or —N═CR₄—, each of the R₄ is independently selected from: H, D, or C₁-C₆ alkyl; ring A is selected from the structure represented by formula (A):

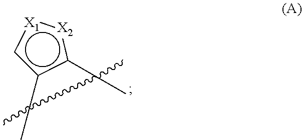

X₁ is independently selected from: C, N, O or S atoms;
X₂ is independently selected from: C, N, O or S atoms;
R₁ is independently selected from: H, D, C₁-C₆ alkyl, C₁-C₆ haloalkyl, C₁-C₆ alkoxy, or C₁-C₆ haloalkoxy;
R₂ is independently selected from: H, D, C₁-C₆ alkyl, wherein the C₁-C₆ alkyl is optionally substituted with one or more halogen, OH, NH₂, or CN group;
Each R₃ is independently selected from: H, D, C₁-C₆ alkyl, C₃-C₆ cycloalkyl, —C(O)NHR₅, wherein the C₁-C₆ alkyl is optionally substituted with one or more halogen, OH, NH₂, CN, or C₁-C₆ alkoxy, the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

In some embodiments of the present invention, in the compound of formula (I), or the pharmaceutically acceptable salt or stereoisomer thereof, X is preferably independently selected from:

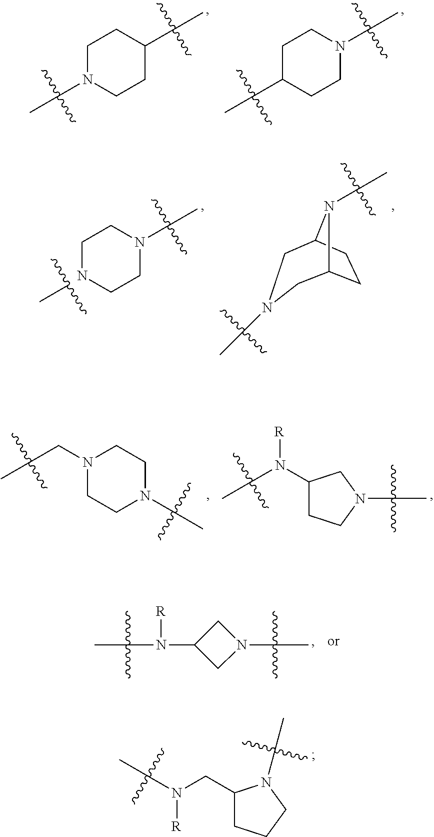

the R is independently selected from: H, D, methyl, ethyl, n-propyl, or isopropyl.

In some embodiments of the present invention, in the compound of formula (I), a pharmaceutically acceptable salt or stereoisomer thereof, Y is preferably independently selected from: divalent group of —CH₂—CH₂—, —CH═CH—, —CH₂—NH—, —NH—CH₂—, —CH₂—N(CH₃)—, —N(CH₃)—CH₂—, —CH═N— or —N═CH—; and more preferably, —CH═CH—.

In some embodiments of the present invention, in the compound of formula (I), a pharmaceutically acceptable salt or stereoisomer thereof, R₁ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, methoxy, ethoxy, n-propoxy, isopropoxy, n-butoxy, sec-butoxy, tert-butoxy, n-pentyloxy, S-pentyloxy, hexyloxy, 2-ethylbutoxy, fluoromethoxy, chloromethoxy, difluoromethoxy, dichloromethoxy, trifluoromethoxy, trichloromethoxy, 2,2-difluoroethoxy, 2,2-dichloroethoxy, 3,3,3-trifluoroethoxy, 3,3,3-trichloroethoxy, pentafluoroethoxy, or pentachloroethoxy.

In some embodiments of the present invention, in the compound of formula (I), a pharmaceutically acceptable salt or stereoisomer thereof, R₂ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl, wherein the terminal position of methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl is optionally substituted with one or more F, Cl, Br, OH, NH₂, or CN group.

In some embodiments of the present invention, in the compound of formula (I), a pharmaceutically acceptable salt or stereoisomer thereof, ring A is preferably selected from a structure represented by formula (A-1), formula (A-2), formula (A-3), formula (A-4) or formula (A-5):

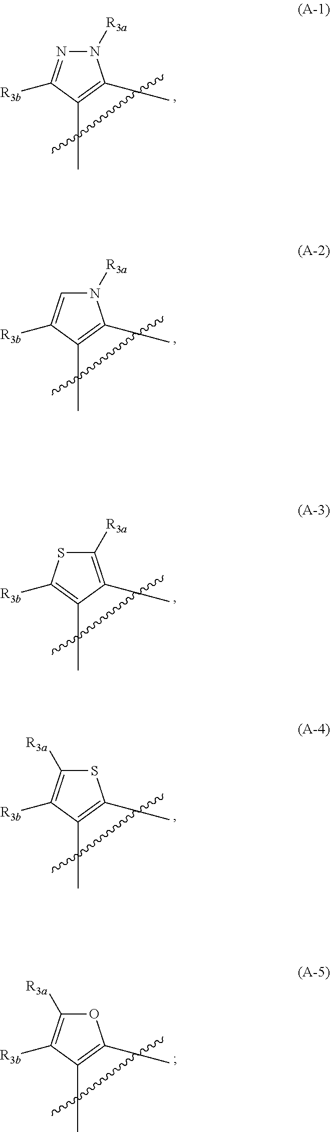

R₃ₐ is independently selected from: H, D, C₁-C₆ alkyl, C₃-C₆ cycloalkyl, wherein the C₁-C₆ alkyl is optionally substituted with one or more halogen, OH, NH₂, CN, or C₁-C₆ alkoxy, R_{3b} is independently selected from: —C(O)NHR₅, the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

In some embodiments of the present invention, in the compound of formula (I), a pharmaceutically acceptable salt or stereoisomer thereof, R₃ₐ is preferably selected from: H, D, C₁-C₆ linear alkyl, C₃-C₆ cycloalkyl; the terminal position of the C₁-C₆ linear alkyl is optionally substituted with one or more F, Cl, Br, OH, NH₂, CN, methoxy, or ethoxy.

In some embodiments of the present invention, in the compound of formula (I), a pharmaceutically acceptable salt or stereoisomer thereof, R₃ₐ is particularly preferably selected from: methyl, ethyl, n-propyl, n-butyl, n-pentyl, n-hexyl, cyclopropyl, cyclobutyl, cyclopentyl or cyclohexyl, wherein the terminal position of the methyl, ethyl, n-propyl, n-butyl, n-pentyl or n-hexyl is optionally substituted with one or more F, Cl, Br, OH, NH₂, CN, methoxy or ethoxy.

In some embodiments of the present invention, in the compound of formula (I), a pharmaceutically acceptable salt or stereoisomer thereof, R_{3b} is independently selected from: —C(O)NHR₅, the R₅ is preferably selected from: H, D, C₁-C₆ linear alkyl, C₁-C₆ linear alkoxy, HO—C₁-C₆ linear alkoxy, OH, NH₂, —NHC(O)NH₂, or R₆—S(O)₂—; the R₆ is independently selected from: C₁-C₆ alkyl, C₁-C₆ haloalkyl, or C₃-C₆ cycloalkyl.

In some embodiments of the present invention, in the compound of formula (I), a pharmaceutically acceptable salt or stereoisomer thereof, R_{3b} is independently selected from: —C(O)NHR₅, the R₅ is particularly preferably selected from: H, D, methyl, ethyl, n-propyl, n-butyl, n-pentyl, n-hexyl, methoxy, ethoxy, n-propoxy, n-butoxy, n-pentyloxy, n-hexyloxy, HO-methoxy, HO-ethoxy, HO-n-propoxy, HO-n-butoxy, HO-n-pentyloxy, HO-n-hexyloxy, OH, NH₂, —NHC(O)NH₂, R₆—S(O)₂—, the R₆ is independently selected from: methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, cyclopropyl, cyclobutyl, cyclopentyl, or cyclohexyl.

In another embodiment of the present invention, there is provided a compound of formula (II), a pharmaceutically acceptable salt or stereoisomer thereof:

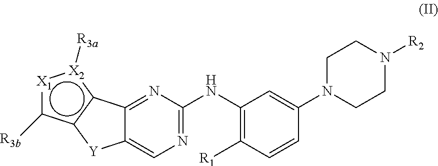

wherein:
Y is independently selected from: divalent group of —C(R₄)₂—C(R₄)₂—, —CR₄═CR₄—, —C(R₄)₂—NR₄—, —NR₄—C(R₄)₂—, —CR₄═N— or —N═CR₄—, each of the R₄ is independently selected from: H, D, or C₁-C₆ alkyl;
X₁ is independently selected from: C, N, O or S atoms;
X₂ is independently selected from: C, N, O or S atoms;
R₁ is independently selected from: H, D, C₁-C₆ alkyl, C₁-C₆ haloalkyl, C₁-C₆ alkoxy, or C₁-C₆ haloalkoxy;
R₂ is independently selected from: H, D, C₁-C₆ alkyl, wherein the C₁-C₆ alkyl is optionally substituted with one or more halogen, OH, NH₂, or CN group;
R₃ₐ is independently selected from: H, D, C₁-C₆ alkyl, or C₃-C₆ cycloalkyl, wherein the C₁-C₆ alkyl is optionally substituted with one or more halogen, OH, NH₂, or CN.
R_{3b} is independently selected from: —C(O)NHR₅,
the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

In some embodiments of the present invention, in the compound of formula (II), a pharmaceutically acceptable salt or stereoisomer thereof, Y is preferably independently selected from: divalent group of —CH₂—CH₂—, —CH═CH—, —CH₂—NH—, —NH—CH₂—, —CH₂—N(CH₃)—, —N(CH₃)—CH₂—, —CH═N— or —N═CH—; and more preferably, —CH═CH—.

In some embodiments of the present invention, in the compound of formula (II), a pharmaceutically acceptable salt or stereoisomer thereof, R₁ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, methoxy, ethoxy, n-propoxy, isopropoxy, n-butoxy, sec-butoxy, tert-butoxy, n-pentyloxy, S-pentyloxy, hexyloxy, 2-ethylbutoxy, fluoromethoxy, chloromethoxy, difluoromethoxy, dichloromethoxy, trifluoromethoxy, trichloromethoxy, 2,2-difluoroethoxy, 2,2-dichloroethoxy, 3,3,3-trifluoroethoxy, 3,3,3-trichloroethoxy, pentafluoroethoxy, or pentachloroethoxy.

In some embodiments of the present invention, in the compound of formula (II), a pharmaceutically acceptable salt or stereoisomer thereof, R₂ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl, wherein the terminal position of methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl is optionally substituted with one or more F, Cl, Br, OH, NH₂, or CN group.

In some embodiments of the present invention, in the compound of formula (II), a pharmaceutically acceptable salt or stereoisomer thereof, R₃ₐ is preferably selected from: H, D, C₁-C₆ linear alkyl, C₃-C₆ cycloalkyl; the terminal position of the C₁-C₆ linear alkyl is optionally substituted with one or more F, Cl, Br, OH, NH₂, or CN group.

In some embodiments of the present invention, in the compound of formula (II), a pharmaceutically acceptable salt or stereoisomer thereof, R₃ₐ is particularly preferably selected from: methyl, ethyl, n-propyl, n-butyl, n-pentyl, n-hexyl, cyclopropyl, cyclobutyl, cyclopentyl or cyclohexyl, wherein the terminal position of the methyl, ethyl, n-propyl, n-butyl, n-pentyl or n-hexyl is optionally substituted with one or more F, Cl, Br, OH, NH₂, or CN group.

In some embodiments of the present invention, in the compound of formula (II), a pharmaceutically acceptable salt or stereoisomer thereof, R_{3b} is independently selected from: —C(O)NHR₅, the R₅ is preferably selected from: H, D, C₁-C₆ linear alkyl, C₁-C₆ linear alkoxy, HO—C₁-C₆ linear alkoxy, OH, NH₂, —NHC(O)NH₂, or R₆—S(O)₂—; the R₆ is independently selected from: C₁-C₆ alkyl, C₁-C₆ haloalkyl, or C₃-C₆ cycloalkyl.

In some embodiments of the present invention, in the compound of formula (II), a pharmaceutically acceptable salt or stereoisomer thereof, R_{3b} is independently selected from: —C(O)NHR₅, the R₅ is particularly preferably selected from: H, D, methyl, ethyl, n-propyl, n-butyl, n-pentyl, n-hexyl, methoxy, ethoxy, n-propoxy, n-butoxy, n-pentyloxy, n-hexyloxy, HO-methoxy, HO-ethoxy, HO-n-propoxy, HO-n-butoxy, HO-n-pentyloxy, HO-n-hexyloxy, OH, NH₂, —NHC(O)NH₂, R₆—S(O)₂—, the R₆ is independently selected from: methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, cyclopropyl, cyclobutyl, cyclopentyl, or cyclohexyl.

In another embodiment of the present invention, there is provided a compound of formula (IIa), formula (IIb), formula (IIc), formula (IId) or formula (IIe), or a pharmaceutically acceptable salt or stereoisomer thereof:

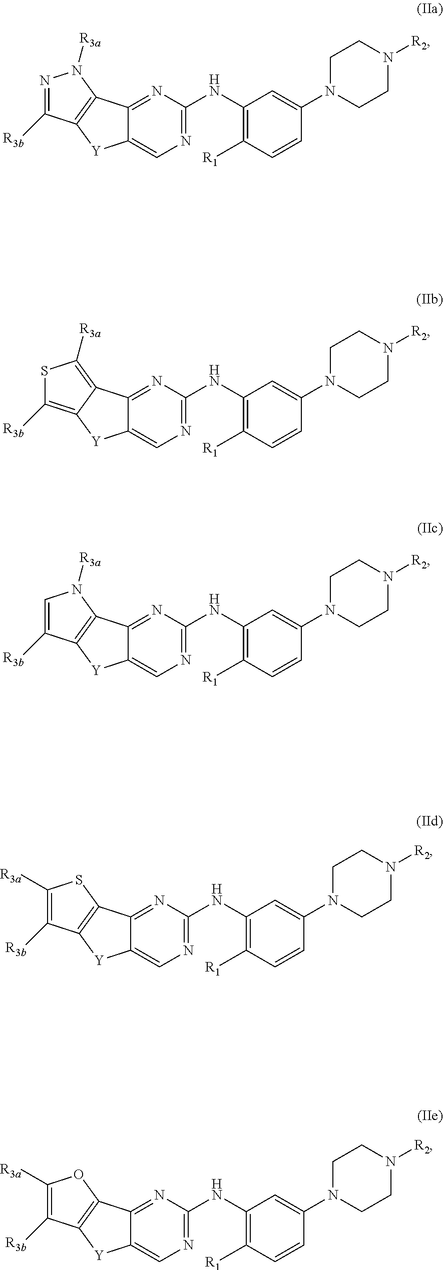

wherein:
Y is independently selected from: divalent group of —C(R₄)₂—C(R₄)₂—, —CR₄═CR₄—, —C(R₄)₂—NR₄—, —NR₄—C(R₄)₂—, —CR₄═N— or —N═CR₄—, each of the R₄ is independently selected from: H, D, or C₁-C₆ alkyl;
R₁ is independently selected from: H, D, C₁-C₆ alkyl, C₁-C₆ haloalkyl, C₁-C₆ alkoxy, or C₁-C₆ haloalkoxy;
R₂ is independently selected from: H, D, C₁-C₆ alkyl, wherein the C₁-C₆ alkyl is optionally substituted with one or more halogen, OH, NH₂, or CN group;
R₃ₐ is independently selected from: H, D, C₁-C₆ alkyl, or C₃-C₆ cycloalkyl, wherein the C₁-C₆ alkyl is optionally substituted with one or more halogen, OH, NH₂, or CN.
R_{3b} is independently selected from: —C(O)NHR₅,
the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

In some embodiments of the present invention, in the compound represented by formula (IIa), formula (IIb), formula (IIc), formula (IId) or formula (IIe), a pharmaceutically acceptable salt or stereoisomer thereof, Y is preferably independently selected from: divalent group of —CH₂—CH₂—, —CH═CH—, —CH₂—NH—, —NH—CH₂—, —CH₂—N(CH₃)—, —N(CH₃)—CH₂—, —CH═N— or —N═CH—; and more preferably, —CH═CH—.

In some embodiments of the present invention, in the compound represented by formula (IIa), formula (IIb), formula (IIc), formula (IId) or formula (IIe), a pharmaceutically acceptable salt or stereoisomer thereof, R₁ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, methoxy, ethoxy, n-propoxy, isopropoxy, n-butoxy, sec-butoxy, tert-butoxy, n-pentyloxy, S-pentyloxy, hexyloxy, 2-ethylbutoxy, fluoromethoxy, chloromethoxy, difluoromethoxy, dichloromethoxy, trifluoromethoxy, trichloromethoxy, 2,2-difluoroethoxy, 2,2-dichloroethoxy, 3,3,3-trifluoroethoxy, 3,3,3-trichloroethoxy, pentafluoroethoxy, or pentachloroethoxy.

In some embodiments of the present invention, in the compound represented by formula (IIa), formula (IIb), formula (IIc), formula (IId) or formula (IIe), a pharmaceutically acceptable salt or stereoisomer thereof, R₂ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl, wherein the terminal position of methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl is optionally substituted with one or more F, Cl, Br, OH, NH₂, or CN group.

In some embodiments of the present invention, in the compound represented by formula (IIa), formula (IIb), formula (IIc), formula (IId) or formula (IIe), a pharmaceutically acceptable salt or stereoisomer thereof, R₃ₐ is preferably selected from: H, D, C₁-C₆ linear alkyl, C₃-C₆ cycloalkyl; the terminal position of the C₁-C₆ linear alkyl is optionally substituted with one or more F, Cl, Br, OH, NH₂, or CN group.

In some embodiments of the present invention, in the compound represented by formula (IIa), formula (IIb), formula (IIc), formula (IId) or formula (IIe), a pharmaceutically acceptable salt or stereoisomer thereof, R₃ₐ is particularly preferably selected from: methyl, ethyl, n-propyl, n-butyl, n-pentyl, n-hexyl, cyclopropyl, cyclobutyl, cyclopentyl or cyclohexyl, wherein the terminal position of the methyl, ethyl, n-propyl, n-butyl, n-pentyl or n-hexyl is optionally substituted with one or more F, Cl, Br, OH, NH₂, or CN group.

In some embodiments of the present invention, in the compound represented by formula (IIa), formula (IIb), formula (IIc), formula (IId) or formula (IIe), a pharmaceutically acceptable salt or stereoisomer thereof, R_{3b} is independently selected from: —C(O)NHR₅, the R₅ is preferably selected from: H, D, C₁-C₆ linear alkyl, C₁-C₆ linear alkoxy, HO—C₁-C₆ linear alkoxy, OH, NH₂, —NHC(O)NH₂, or R₆—S(O)₂—; the R₆ is independently selected from: C₁-C₆ alkyl, C₁-C₆ haloalkyl, or C₃-C₆ cycloalkyl.

In some embodiments of the present invention, in the compound represented by formula (IIa), formula (IIb), formula (IIc), formula (IId) or formula (IIe), a pharmaceutically acceptable salt or stereoisomer thereof, R_{3b} is independently selected from: —C(O)NHR₅, the R₅ is particularly preferably selected from: H, D, methyl, ethyl, n-propyl, n-butyl, n-pentyl, n-hexyl, methoxy, ethoxy, n-propoxy, n-butoxy, n-pentyloxy, n-hexyloxy, HO-methoxy, HO-ethoxy, HO-n-propoxy, HO-n-butoxy, HO-n-pentyloxy, HO-n-hexyloxy, OH, NH₂, —NHC(O)NH₂, R₆—S(O)₂—, the R₆ is independently selected from: methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, cyclopropyl, cyclobutyl, cyclopentyl, or cyclohexyl.

In some embodiments of the present invention, in the compound of formula (IIa), a pharmaceutically acceptable salt or stereoisomer thereof, Y is —CR₄═CR₄—; Each of the R₄ is independently selected from: H, D, or C₁-C₆ alkyl; preferably Y is —CH═CH—.

R₁ is independently selected from: H, D, C₁-C₆ alkyl, C₁-C₆ haloalkyl, C₁-C₆ alkoxy, or C₁-C₆ haloalkoxy.

R₂ is independently selected from: H, D, C₁-C₆ alkyl, wherein the C₁-C₆ alkyl is optionally substituted with one or more halogen, OH, NH₂, or CN group.

R₃ₐ is independently selected from: H, D, C₁-C₆ alkyl, or C₃-C₆ cycloalkyl, wherein the C₁-C₆ alkyl is optionally substituted with one or more halogen, OH, NH₂, or CN.

R_{3b} is independently selected from: —C(O)NHR₅, the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

In some embodiments of the present invention, in the compound of formula (IIa), a pharmaceutically acceptable salt or stereoisomer thereof, R₁ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, methoxy, ethoxy, n-propoxy, isopropoxy, n-butoxy, sec-butoxy, tert-butoxy, n-pentyloxy, S-pentyloxy, hexyloxy, 2-ethylbutoxy, fluoromethoxy, chloromethoxy, difluoromethoxy, dichloromethoxy, trifluoromethoxy, trichloromethoxy, 2,2-difluoroethoxy, 2,2-dichloroethoxy, 3,3,3-trifluoroethoxy, 3,3,3-trichloroethoxy, pentafluoroethoxy, or pentachloroethoxy.

In some embodiments of the present invention, in the compound of formula (IIa), a pharmaceutically acceptable salt or stereoisomer thereof, R₂ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl, wherein the terminal position of methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl is optionally substituted with one or more F, Cl, Br, OH, NH₂, or CN group.

In some embodiments of the present invention, in the compound of formula (IIa), a pharmaceutically acceptable salt or stereoisomer thereof, R₃ₐ is preferably selected from: H, D, C₁-C₆ linear alkyl, C₃-C₆ cycloalkyl; the terminal position of the C₁-C₆ linear alkyl is optionally substituted with one or more F, Cl, Br, OH, NH₂, or CN group.

In some embodiments of the present invention, in the compound of formula (IIa), a pharmaceutically acceptable salt or stereoisomer thereof, R₃ₐ is preferably selected from: methyl, ethyl, n-propyl, n-butyl, n-pentyl, n-hexyl, cyclopropyl, cyclobutyl, cyclopentyl or cyclohexyl, wherein the terminal position of the methyl, ethyl, n-propyl, n-butyl, n-pentyl or n-hexyl is optionally substituted with one or more F, Cl, Br, OH, NH₂, or CN group; R₃ₐ is particularly preferably selected from methyl, ethyl, cyclopropyl, —CH₂CH₂F, —CH₂CF₃, or —CH₂CH₂OH.

In some embodiments of the present invention, in the compound of formula (IIa), a pharmaceutically acceptable salt or stereoisomer thereof, R_{3b} is independently selected from: —C(O)NHR₅, the R₅ is preferably selected from: H, D, C₁-C₆ linear alkyl, C₁-C₆ linear alkoxy, HO—C₁-C₆ linear alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

In some embodiments of the present invention, in the compound of formula (IIa), a pharmaceutically acceptable salt or stereoisomer thereof, R_{3b} is independently selected from: —C(O)NHR₅, the R₅ is particularly preferably selected from: H, D, methyl, ethyl, n-propyl, n-butyl, n-pentyl, n-hexyl, methoxy, ethoxy, n-propoxy, n-butoxy, n-pentyloxy, n-hexyloxy, HO-methoxy, HO-ethoxy, HO-n-propoxy, HO-n-butoxy, HO-n-pentyloxy, HO-n-hexyloxy, OH, NH₂, —NHC(O)NH₂, R₆—S(O)₂—, the R₆ is independently selected from: methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, cyclopropyl, cyclobutyl, cyclopentyl, or cyclohexyl.

In some aspects of the present invention, the compound of formula (I) is selected from:

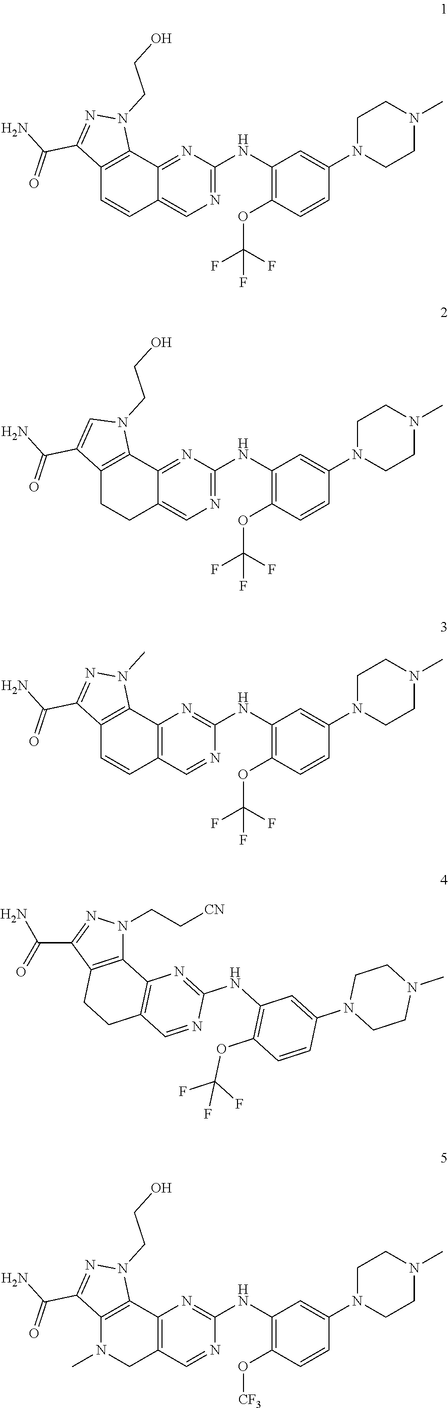 [SMILES: CN1CCN(c2ccc(OC(F)(F)F)c(Nc3ncc4ccc5c(C(N)=O)nn(CCO)c5c4n3)c2)CC1 ; CN1CCN(c2ccc(OC(F)(F)F)c(Nc3ncc4c(n3)-c3c(c(C(N)=O)cn3CCO)CC4)c2)CC1 ; CN1CCN(c2ccc(OC(F)(F)F)c(Nc3ncc4ccc5c(C(N)=O)nn(C)c5c4n3)c2)CC1 ; CN1CCN(c2ccc(OC(F)(F)F)c(Nc3ncc4c(n3)-c3c(c(C(N)=O)nn3CCC#N)CC4)c2)CC1 ; CN1CCN(c2ccc(OC(F)(F)F)c(Nc3ncc4c(n3)-c3c(c(C(N)=O)nn3CCO)N(C)C4)c2)CC1]

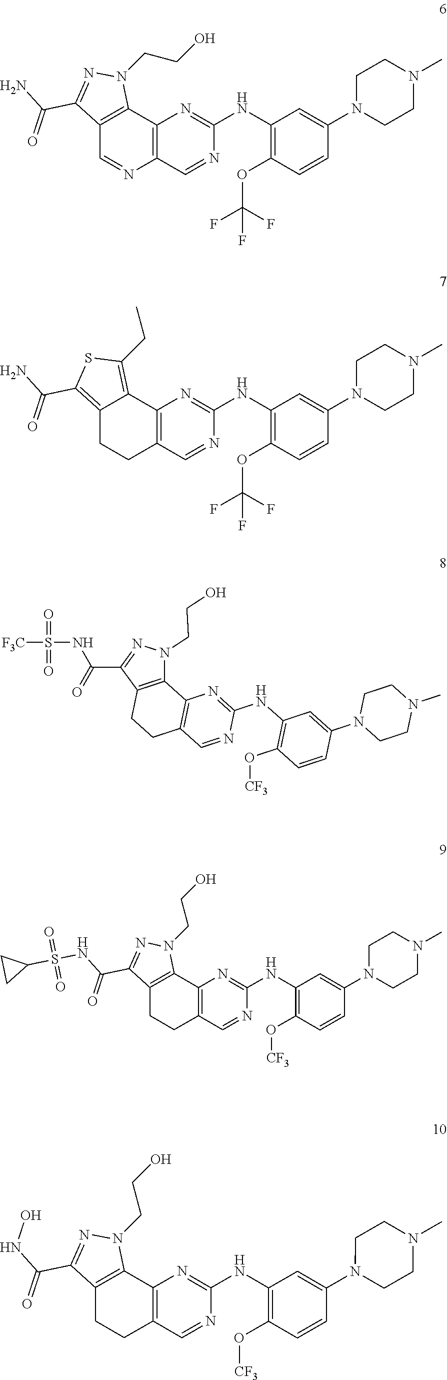 [SMILES: CN1CCN(c2ccc(OC(F)(F)F)c(Nc3ncc4ncc5c(C(N)=O)nn(CCO)c5c4n3)c2)CC1 ; CCc1sc(C(N)=O)c2c1-c1nc(Nc3cc(N4CCN(C)CC4)ccc3OC(F)(F)F)ncc1CC2 ; CN1CCN(c2ccc(OC(F)(F)F)c(Nc3ncc4c(n3)-c3c(c(C(=O)NS(=O)(=O)C(F)(F)F)nn3CCO)CC4)c2)CC1 ; CN1CCN(c2ccc(OC(F)(F)F)c(Nc3ncc4c(n3)-c3c(c(C(=O)NS(=O)(=O)C5CC5)nn3CCO)CC4)c2)CC1 ; CN1CCN(c2ccc(OC(F)(F)F)c(Nc3ncc4c(n3)-c3c(c(C(=O)NO)nn3CCO)CC4)c2)CC1]

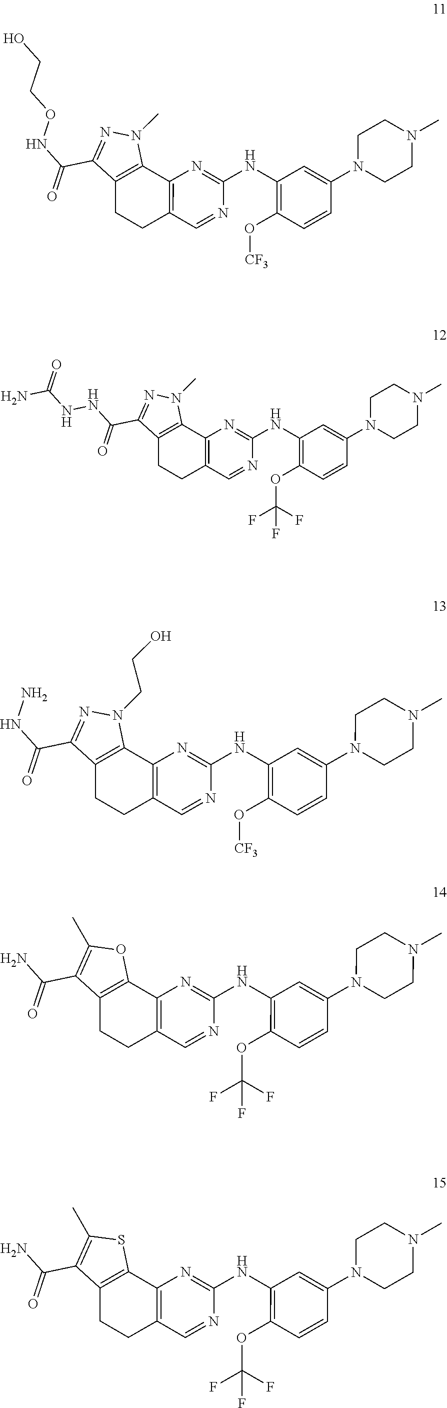 [SMILES: CN1CCN(c2ccc(OC(F)(F)F)c(Nc3ncc4c(n3)-c3c(c(C(=O)NOCCO)nn3C)CC4)c2)CC1 ; CN1CCN(c2ccc(OC(F)(F)F)c(Nc3ncc4c(n3)-c3c(c(C(=O)NNC(N)=O)nn3C)CC4)c2)CC1 ; CN1CCN(c2ccc(OC(F)(F)F)c(Nc3ncc4c(n3)-c3c(c(C(=O)NN)nn3CCO)CC4)c2)CC1 ; Cc1oc2c(c1C(N)=O)CCc1cnc(Nc3cc(N4CCN(C)CC4)ccc3OC(F)(F)F)nc1-2 ; Cc1sc2c(c1C(N)=O)CCc1cnc(Nc3cc(N4CCN(C)CC4)ccc3OC(F)(F)F)nc1-2]

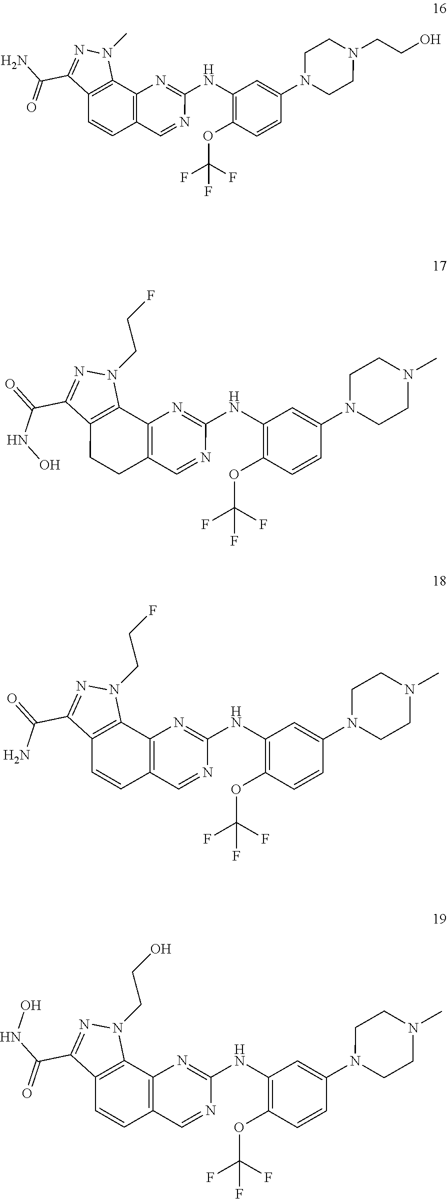 [SMILES: Cn1nc(C(N)=O)c2ccc3cnc(Nc4cc(N5CCN(CCO)CC5)ccc4OC(F)(F)F)nc3c21 ; CN1CCN(c2ccc(OC(F)(F)F)c(Nc3ncc4c(n3)-c3c(c(C(=O)NO)nn3CCF)CC4)c2)CC1 ; CN1CCN(c2ccc(OC(F)(F)F)c(Nc3ncc4ccc5c(C(N)=O)nn(CCF)c5c4n3)c2)CC1 ; CN1CCN(c2ccc(OC(F)(F)F)c(Nc3ncc4ccc5c(C(=O)NO)nn(CCO)c5c4n3)c2)CC1]

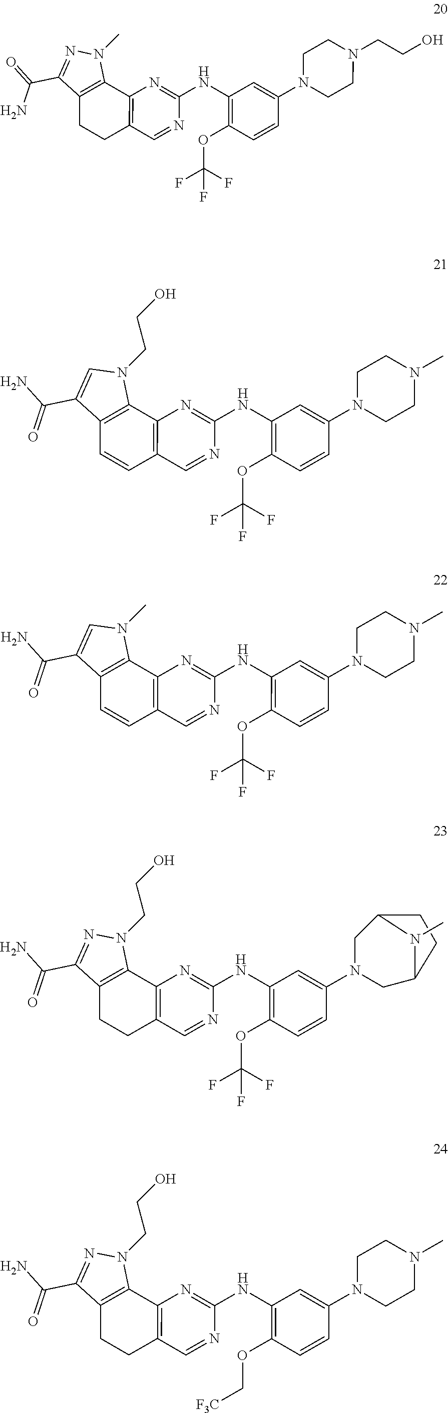 [SMILES: Cn1nc(C(N)=O)c2c1-c1nc(Nc3cc(N4CCN(CCO)CC4)ccc3OC(F)(F)F)ncc1CC2 ; CN1CCN(c2ccc(OC(F)(F)F)c(Nc3ncc4ccc5c(C(N)=O)cn(CCO)c5c4n3)c2)CC1 ; CN1CCN(c2ccc(OC(F)(F)F)c(Nc3ncc4ccc5c(C(N)=O)cn(C)c5c4n3)c2)CC1 ; CN1C2CCC1CN(c1ccc(OC(F)(F)F)c(Nc3ncc4c(n3)-c3c(c(C(N)=O)nn3CCO)CC4)c1)C2 ; CN1CCN(c2ccc(OCC(F)(F)F)c(Nc3ncc4c(n3)-c3c(c(C(N)=O)nn3CCO)CC4)c2)CC1]

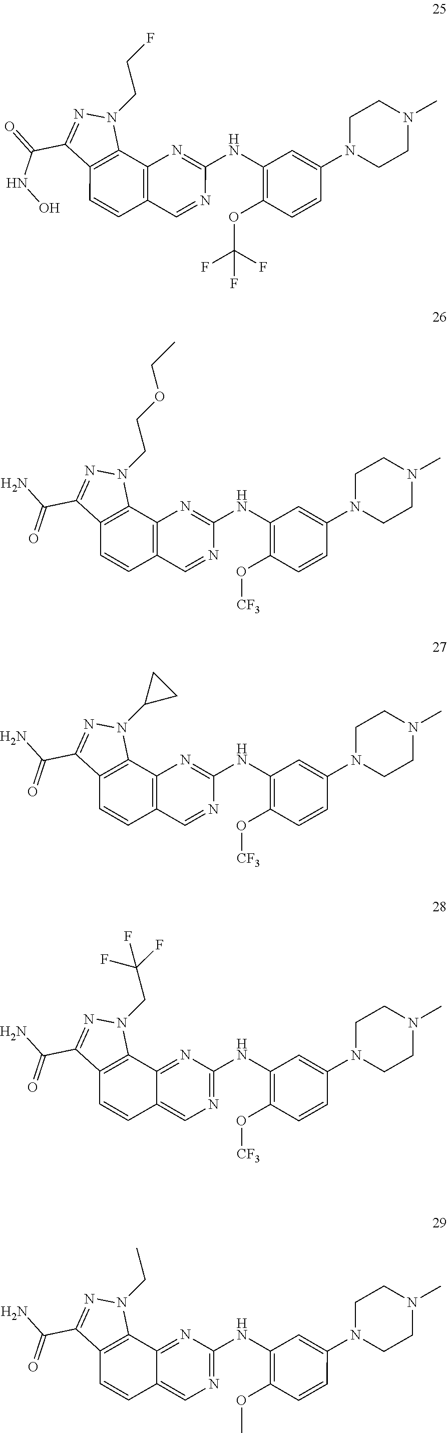 [SMILES: CN1CCN(c2ccc(OC(F)(F)F)c(Nc3ncc4ccc5c(C(=O)NO)nn(CCF)c5c4n3)c2)CC1 ; CCOCCn1nc(C(N)=O)c2ccc3cnc(Nc4cc(N5CCN(C)CC5)ccc4OC(F)(F)F)nc3c21 ; CN1CCN(c2ccc(OC(F)(F)F)c(Nc3ncc4ccc5c(C(N)=O)nn(C6CC6)c5c4n3)c2)CC1 ; CN1CCN(c2ccc(OC(F)(F)F)c(Nc3ncc4ccc5c(C(N)=O)nn(CC(F)(F)F)c5c4n3)c2)CC1 ; CCn1nc(C(N)=O)c2ccc3cnc(Nc4cc(N5CCN(C)CC5)ccc4OC)nc3c21]

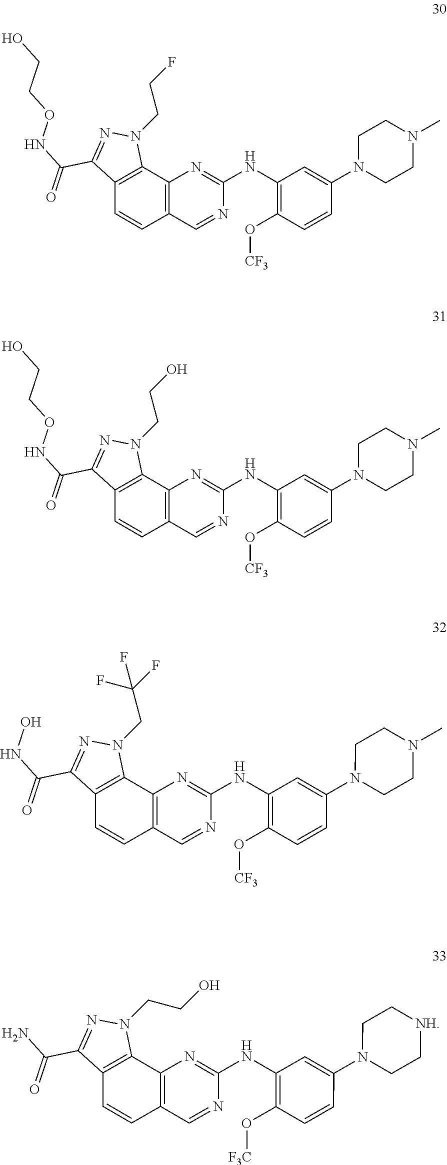 [SMILES: CN1CCN(c2ccc(OC(F)(F)F)c(Nc3ncc4ccc5c(C(=O)NOCCO)nn(CCF)c5c4n3)c2)CC1 ; CN1CCN(c2ccc(OC(F)(F)F)c(Nc3ncc4ccc5c(C(=O)NOCCO)nn(CCO)c5c4n3)c2)CC1 ; CN1CCN(c2ccc(OC(F)(F)F)c(Nc3ncc4ccc5c(C(=O)NO)nn(CC(F)(F)F)c5c4n3)c2)CC1 ; NC(=O)c1nn(CCO)c2c1ccc1cnc(Nc3cc(N4CCNCC4)ccc3OC(F)(F)F)nc12]

The present invention also provides a pharmaceutical composition, including any one of the above compounds or the pharmaceutically acceptable salt thereof and a pharmaceutically acceptable carrier. The pharmaceutical composition can be prepared into various pharmaceutically acceptable dosage forms, such as tablets, capsules, oral liquid preparations, granules, injections or various sustained and controlled release preparations. The pharmaceutical composition can be administered by oral administration or parenteral mode (such as intravenous, subcutaneous or topically). Dosage of administration can be appropriately adjusted according to the age, gender and disease type of the patients, and the daily dosage is generally about 1-200 mg. The present invention also provides the use of the above-mentioned compound or the pharmaceutically acceptable salt thereof or the pharmaceutical composition in preparing a drug for the prevention or treatment of diseases caused by and/or related to dysregulation of protein kinase activity, preferably in preparing a drug for the prevention or treatment of diseases caused by and/or related to dysregulation of PLK1 kinase activity. The disease is selected from a cancer, a cell proliferative disease, a viral infection, an autoimmune and neurodegenerative disease.

Such a cancer includes, but not limited to: a cancer such as bladder cancer, breast cancer, colon cancer, kidney cancer, liver cancer, lung cancer (including small cell lung cancer), esophageal cancer, gallbladder cancer, ovarian cancer, pancreatic cancer, gastric cancer, cervical cancer, thyroid cancer, prostate cancer and skin cancer (including squamous cell cancer); hematopoietic tumors of the lymphoid system, including leukemia, acute lymphocitic leukemia, acute lymphoblastic leukemia, B-cell lymphoma, T-cell lymphoma, Hodgkin's lymphoma, non-Hodgkin's lymphoma, hairy cell lymphoma, and Burkett's lymphoma; hematopoietic tumors of the myeloid system, including acute and chronic myeloid leukemias, myelodysplastic syndromes, and promyelocytic leukemia; tumors of mesenchymal origin, including fibrosarcoma and rhabdomyosarcoma; tumors of the central and peripheral nervous system, including astrocytomas, neuroblastomas, gliomas, and schwannomas; other tumors, including melanoma, seminoma, teratoma, osteosarcoma, xeroderma pigmentosum, keratoacanthoma, follicular thyroid carcinoma, and Kaposi's sarcoma.

Such cell proliferative diseases are such as benign prostatic hyperplasia, familial adenoma, polyposis, neurofibromatosis, psoriasis, vascular smooth cell proliferation associated with atherosclerosis, pulmonary fibrosis, arthritis, glomerulonephritis, and postoperative stenosis and restenosis.

### Definition and Description

Unless otherwise stated, the following terms and phrases used herein are intended to have the following meanings. A specific term or phrase should not be considered uncertain or unclear unless specifically defined, but should be understood in its ordinary meaning. When a trade name appears herein, it is intended to refer to the corresponding commodity or an active ingredient thereof.

The “pharmaceutically acceptable” in the present invention refers to compounds, compositions and/or dosage forms, which are, within the scope of sound medical judgment, suitable for use in contact with human and animal tissues, without excessive toxicity, irritation, allergic reactions or other problems or complications, which is commensurate with a reasonable benefit/risk ratio.

The “pharmaceutically acceptable salt” in the present invention refers to a salt of the compound of the present invention, which is prepared from the compound having specific substituents found in the present invention with relatively non-toxic acids or bases. When compounds of the present invention contain relatively basic functional groups, acid addition salts can be obtained by contacting the neutral form of such compounds with a sufficient amount of acid, either in pure solution or a suitable inert solvent. Examples of pharmaceutically acceptable acid addition salts include inorganic acid salts or organic acid salts; and also include salts of amino acids (such as arginine), and salts of organic acids such as glucuronic acid. Certain specific compounds of the present invention contain acidic functional groups and thus can be converted to any acid addition salt.

Certain compounds of the present invention may have asymmetric carbon atoms (optical centers) or double bonds. Racemates, diastereomers, geometric isomers and individual isomers are encompassed within the scope of the present invention.

The compounds of the present invention may exist in specific geometric or stereoisomeric forms. The present invention contemplates all such compounds, including cis and trans isomers, (−)- and (+)-enantiomers, (R)- and (S)-enantiomers, diastereomers, (D)-isomers, (L)-isomers, and racemic mixtures and other mixtures thereof, such as enantiomerically or diastereomerically enriched mixtures, all of which fall within the scope of the present invention. Additional asymmetric carbon atoms may be present in a substituent such as an alkyl group. All these isomers and mixtures thereof are encompassed within the scope of the present invention.

The pharmaceutically acceptable salts of the present invention can be synthesized from a parent compound containing acid radicals or base radicals by conventional chemical methods. In general, a method for preparing such salts comprises: in water or an organic solvent or a mixture of both, reacting these compounds in free acid or base forms with a stoichiometric amount of a suitable base or acid to prepare the salts.

The term “pharmaceutically acceptable carrier” refers to any preparation or carrier medium that can deliver an effective amount of active substances in the present invention, does not interfere with the biological activity of the active substances, and has no toxic or side effects on a host or patient. Representative carriers include, but are not limited to: a binder, a filler, a lubricant, a disintegrant, a wetting agent, a dispersing agent, a solubilizer, a suspending agent, etc.

The present invention is intended to include all isotopes of atoms present in the compounds of the present invention. The isotopes comprise those atoms with the same atomic number but different mass numbers. By way of general example, and not as a limitation, isotopes of hydrogen comprise deuterium and tritium. Isotopes of carbon comprise ¹³C and ¹⁴C. Isotope-labeled compounds of the present invention can generally be prepared by using an appropriate isotope-labeled reagent in place of a non-labeled reagent otherwise used by means of conventional techniques known to a person skilled in the art or by means of methods analogous to those described herein.

Unless otherwise specified, the term “alkyl” is used to represent a linear or branched saturated hydrocarbon group, which may be monosubstituted (e.g., —CH₂F) or polysubstituted (e.g., —CF₃) and may be monovalent (e.g., methyl), divalent (e.g., methylene) or polyvalent (e.g., methine). For example, C₁-C₆ represents 1 to 6 carbons, C₁₋₆ is selected from C₁, C₂, C₃, C₄, C₅, or C₆; Examples of alkyl include methyl (Me), ethyl (Et), propyl (e.g., n-propyl and isopropyl), butyl (e.g., n-butyl, isobutyl, s-butyl and t-butyl), pentyl (e.g., n-pentyl, isopentyl, neopentyl and 1-ethylpropyl), hexyl (e.g., n-hexyl, isohexyl, 1,1-dimethylbutyl, 2,2-dimethylbutyl, 3,3-dimethylbutyl and 2-ethylbutyl), etc.

Unless otherwise specified, the term “halo” or “halogen” by itself or as part of another substituent means a fluorine, chlorine, bromine or iodine atom.

“Haloalkyl” is used to represent a monohalogenated/polyhalogenated linear or branched alkyl group. Typical haloalkyl includes C₁₋₆ haloalkyl, for example: C₁, C₂, C₃, C₄, C₅, or C₆ haloalkyl. For example, examples of C₁-C₆ haloalkyl includes but not limited to: fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, and pentachloroethyl.

Unless otherwise specified, the “alkoxy” represents the above alkyl (including cycloalkyl or haloalkyl) having a specific number of carbon atoms and connected via an oxygen bridge. Typical alkoxy includes C₁₋₆ alkoxy, such as: C₁, C₂, C₃, C₄, C₅, or C₆ alkoxy, C₃, C₄, C₅, or C₆ cycloalkoxy, C₁, C₂, C₃, C₄, C₅, or C₆ haloalkoxy. Examples of alkoxy include, but are not limited to: methoxy, ethoxy, n-propoxy, isopropoxy, n-butoxy, sec-butoxy, tert-butoxy, n-pentoxy, S-pentoxy, hexyloxy, or 2-ethylbutoxy. Examples of cycloalkoxy include, but are not limited to: cyclopropoxy, cyclobutoxy, cyclopentoxy, and cyclohexoxy. Examples of haloalkoxy include, but are not limited to: fluoromethoxy, chloromethoxy, difluoromethoxy, dichloromethoxy, trifluoromethoxy, trichloromethoxy, 2,2-difluoroethoxy, 2,2-dichloroethoxy, 3,3,3-trifluoroethoxy, 3,3,3-trichloroethoxy, pentafluoroethoxy, or pentachloroethoxy.

Unless otherwise specified, cycloalkyl includes any stable cyclic or polycyclic hydrocarbon group, and any carbon atom is saturated, which may be monosubstituted or polysubstituted and may be monovalent, divalent or polyvalent. Examples of these cycloalkyl groups include, but are not limited to, cyclopropyl, cyclobutyl, cyclopentyl, cyclohexyl, and the like.

Compounds are named by hand or ChemDraw® software, and commercially available compounds are named by the supplier catalog names.

### BRIEF DESCRIPTION OF THE DRAWINGS

FIG. 1 shows in vivo pharmacodynamic study of compound 1 in mice—tumor growth curve in animal.

FIG. 2 shows in vivo pharmacodynamic study of compound 1 in mice—body weight during test in animal.

FIG. 3 shows in vivo pharmacodynamic study of compound 18 in mice—tumor growth curve in animal.

FIG. 4 shows in vivo pharmacodynamic study of compound 18 in mice—body weight during test in animal.

### DETAILED DESCRIPTION OF EMBODIMENTS

The present invention is further described below in conjunction with specific examples and test examples, but the scope of the present invention is not limited in any way.

In the following examples, the following reagent abbreviations have the following meanings. If not defined, they have meanings well-known in the art.

| Abbreviations | Reagent meaning/Chinese name |
|---|---|
| PTSA | sodium p-toluenesulfonate |
| LiHMDS | lithium bis(trimethylsily1)amide |
| DMF-DMA | N,N-dimethylformamide dimethyl acetal |
| DMF | N,N-dimethylformamide |
| TFA | trifluoroacetic acid |
| TEMPO | 2,2,6,6-tetramethylpiperidine oxide |
| TosMIC | p-toluenesulfonylmethyl isonitrile |
| TFAA | trifluoroacetic anhydride |
| HOBt | 1-hydroxybenzotriazole |
| DIEA | N,N-diisopropylethylamine |
| EDCI | 1-ethyl-(3-dimethylaminopropyl)carbodiimide |
| | hydrochloride |
| DDQ | 2,3-dichloro-5,6-dicyano-1,4-benzoquinone |
| CAN | ceric ammonium nitrate |
| NMP | N-methylpyrrolidone |
| DMAP | 4-(dimethylamino)pyridine |
| CDI | 1,1′-carbonyldiimidazole |
| DAST | diethylamine sulfur trifluoride |
| HATU | 2-(7-azabenzotriazol-1-yl)-N,N,N′,N′- |
| | tetramethyluronium hexafluorophosphate |
| TBDPSCl | tert-butyldiphenylchlorosilane |
| DavePhos | 2-dicyclohexylphosphino-2′-(N,N- |
| | dimethylamine)biphenyl |
| Pd2(dba)3 | tris(dibenzylideneacetone)dipalladium |
| KHMDS | potassium bis(trimethylsilyl)amide |
| TMSCl | trimethylchlorosilane |
| BFMO | N1,N2-bis(furan-2-ylmethyl)oxalamide |
| DCM | dichloromethane |

### Example 1: Synthesis of Compound 1

### Synthesis Route:

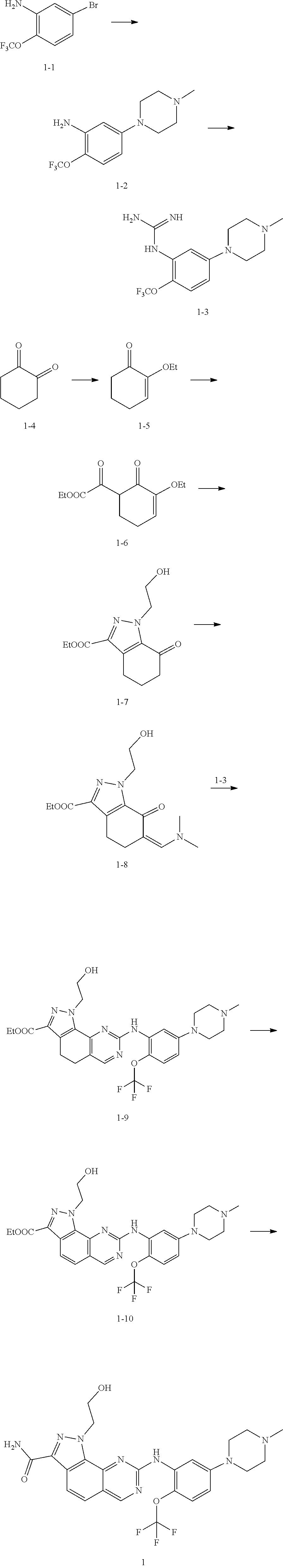

### 1.1 Synthesis of Intermediate 1-2

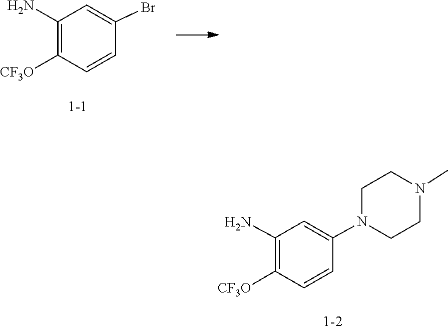

Intermediate 1-1 (20.0 g, 78.1 mmol), bis(dibenzylideneacetone)palladium (0.715 g, 0.78 mmol), and 2-dicyclohexylphosphino-2′-(N,N-dimethylamine)-biphenyl (0.615 g, 1.56 mmol) were dissolved in tetrahydrofuran (32 mL), and N-methylpiperazine (12.5 g, 125 mmol) and lithium bis(trimethylsilyl)amide (1.00 M, 187 mL) were added at 0° C. The mixture was reacted at 70° C. for 3 h. Water (200 mL) was added to quench the reaction, and dichloromethane (100 mL×3) was added for extraction. The organic phases were combined, dried over anhydrous sodium sulfate, filtered, and concentrated under reduced pressure. The residue was subjected to silica gel column chromatography (methanol/dichloromethane=1%-10%) to obtain intermediate 1-2 (15.5 g, yield 72.2%, brown solid). MS (ESI) m/z=276.0 [M+H]⁺.

### 1.2 Synthesis of Intermediate 1-3

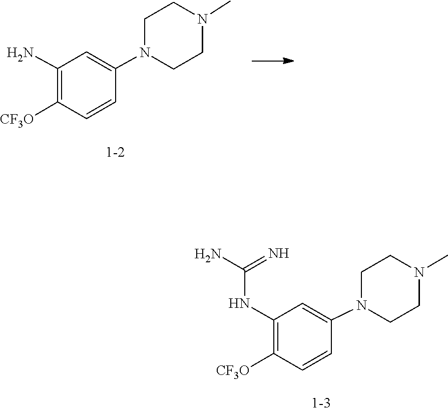

Intermediate 1-2 (0.372 g, 1.35 mmol) was dissolved in 6 N HCl (2 mL), and cyanamide (0.455 g, 10.8 mmol) was added. The mixture was reacted at 80° C. for 12 h. The reaction liquid was concentrated under reduced pressure, and the residue was subjected to column chromatography (C18, acetonitrile/water=0%-10%). The intermediate 1-3 (0.19 g, yield 45%, brown solid) was obtained. MS (ESI) m/z=318.1 [M+H]⁺.

### 1.3 Synthesis of Intermediate 1-5

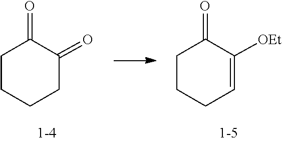 [SMILES: O=C1CCCCC1=O ; CCOC1=CCCCC1=O]

Intermediate 1-4 (100 g, 892 mmol) was dissolved in toluene (1000 mL) and ethanol (123 g, 2680 mmol), PTSA (15.4 g, 89.2 mmol) was added, and the mixture was reacted at 120° C. for 12 h. Water (1000 mL×2) was added for extraction. The organic phases were collected, dried over anhydrous sodium sulfate, filtered, and the organic phase was concentrated under reduced pressure. The residue was subjected to silica gel column chromatography (ethyl acetate/petroleum ether=0%-50%) to obtain intermediate 1-5 (57.9 g, yield 46.4%, oil).

### Synthesis of Intermediate 1-6

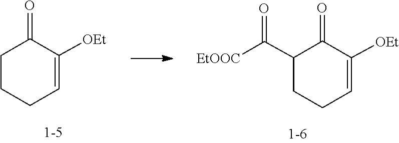 [SMILES: CCOC1=CCCCC1=O ; CCOC(=O)C(=O)C1CCC=C(OCC)C1=O]

Intermediate 1-5 (30.0 g, 214 mmol) was dissolved in tetrahydrofuran (450 mL), LiHMDS (1 M, 256 mL) was added dropwise at −50.0° C., and the mixture was stirred for 30 min. Diethyl oxalate (37.8 g, 258 mmol) was added and the mixture was reacted at room temperature for 16 h. Saturated ammonium chloride (500 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-20%) to obtain intermediate 1-6 (25.0 g, yield 48.6%, oil). MS (ESI) m/z=241.1 [M+H]⁺.

### 1.5 Synthesis of Intermediate 1-7

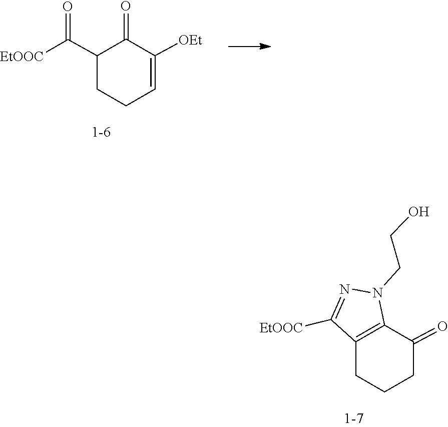 [SMILES: CCOC(=O)C(=O)C1CCC=C(OCC)C1=O ; CCOC(=O)c1nn(CCO)c2c1CCCC2=O]

Intermediate 1-6 (25.0 g, 104 mmol) was dissolved in acetic acid (120 mL), hydroxyethylhydrazine (8.16 g, 107 mmol) was added, and the mixture was reacted at room temperature for 15 h. water (500 mL) was added to quench the reaction, saturated Na₂CO₃ (800 mL) was added, and dichloromethane (800 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-5%) to obtain intermediate 1-7 (19.9 g, yield 76%, yellow solid). MS (ESI) m/z=253.0 [M+H]⁺.

### 1.6 Synthesis of Intermediate 1-8

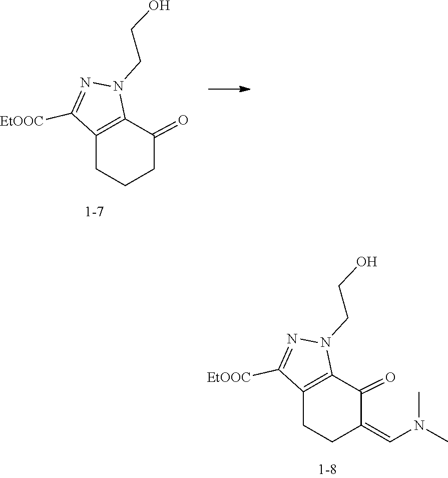 [SMILES: CCOC(=O)c1nn(CCO)c2c1CCCC2=O ; CCOC(=O)c1nn(CCO)c2c1CC/C(=C/N(C)C)C2=O]

Intermediate 1-7 (10.0 g, 39.6 mmol) was dissolved in DMF-DMA (100 mL) and the mixture was reacted at 110° C. for 12 h. The reaction liquid was concentrated under reduced pressure, and the residue was dissolved in methyl tertiary ether and the mixture was stirred for 3 h. The resulting product was filtered, the filter cake was collected, and dried to obtain intermediate 1-8 (6.0 g, crude product), which was directly used in the next step.

### 1.7 Synthesis of Intermediate 1-9

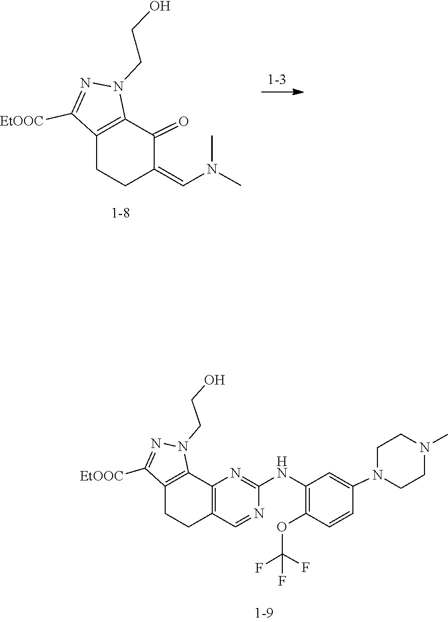 [SMILES: CCOC(=O)c1nn(CCO)c2c1CC/C(=C/N(C)C)C2=O ; CCOC(=O)c1nn(CCO)c2c1CCc1cnc(Nc3cc(N4CCN(C)CC4)ccc3OC(F)(F)F)nc1-2]

Intermediate 1-8 (1.95 g, 6.34 mmol) and intermediate 1-3 (2.03 g, 6.41 mmol) were dissolved in DMF (20 mL) and the mixture was reacted at 110° C. for 12 h. The reaction liquid was concentrated under reduced pressure, and the residue was subjected to silica gel column chromatography (ethyl acetate/petroleum ether=0%-5%) to obtain intermediate 1-9 (1.90 g, yield 53.3%, yellow solid). MS (ESI) m/z=562.2 [M+H]⁺.

### 1.8 Synthesis of Intermediate 1-10

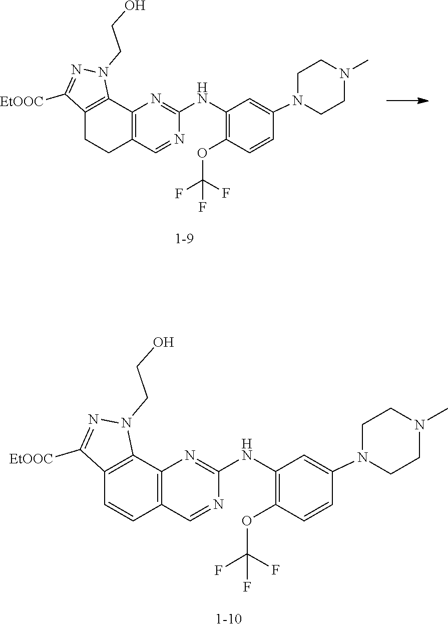 [SMILES: CCOC(=O)c1nn(CCO)c2c1CCc1cnc(Nc3cc(N4CCN(C)CC4)ccc3OC(F)(F)F)nc1-2 ; CCOC(=O)c1nn(CCO)c2c1ccc1cnc(Nc3cc(N4CCN(C)CC4)ccc3OC(F)(F)F)nc12]

Intermediate 1-9 (100 mg, 178 μmol) and DDQ (100 mg, 440 μmol) were dissolved in dioxane (20 mL) and the mixture was reacted at 100° C. for 12 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (C18 150*40 mm*10 μm; mobile phase: [water (TFA)-acetonitrile]; B %: 25%-55%, 10 min). Intermediate 1-10 (10 mg, yield 10%, brown solid) was obtained. MS (ESI) m/z=560.1 [M+H]⁺.

### 1.9 Synthesis of Compound 1

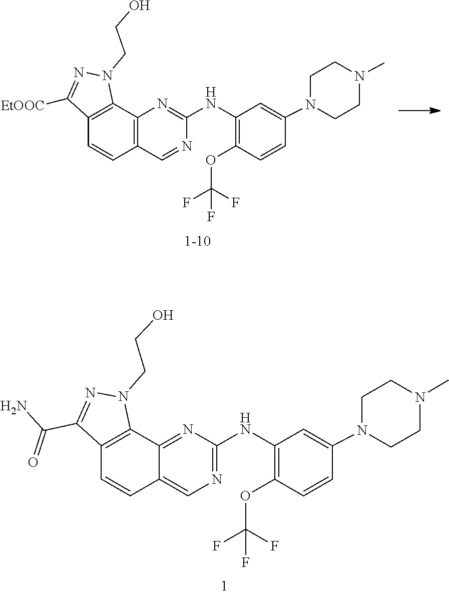 [SMILES: CCOC(=O)c1nn(CCO)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(N)=O)nn(CCO)c5c4n3)c2)CC1]

Intermediate 1-10 (160 mg, 285 μmol) was dissolved in NMP (6 mL), and NH₃/MeOH (50.0 mL) was added. The mixture was reacted for 48 h under sealed conditions at 80.0° C. The reaction liquid was concentrated under reduced pressure, and the residue was purified by preparative liquid chromatogram (C18 30*150 mm*7 μm; mobile phase: [water (TFA)-acetonitrile]; B %: 10%-80%, 40 min). Compound 1 (56.7 mg, yield 37.5%, yellow solid) was obtained. MS (ESI) m/z=531.2 [M+H]. ¹H NMR (400 MHz, DMSO-d6) δ 9.56 (s, 1H), 9.34 (s, 1H), 7.89 (d, J=8.6 Hz, 1H), 7.67 (d, J=8.8 Hz, 1H), 7.33-7.20 (m, 2H), 6.88-6.91 (m, 1H), 4.91 (t, J=4.9 Hz, 2H), 4.65 (d, J=5.3 Hz, 1H), 4.41 (q, J=7.1 Hz, 2H), 3.70-3.54 (m, 3H), 3.28-3.10 (m, 3H), 3.10-3.09 (m, 1H), 3.16-3.09 (m, 1H), 2.34-2.19 (m, 3H), 1.39 (t, J=7.1 Hz, 3H), 1.36-1.36 (m, 1H), 1.31 (t, J=7.1 Hz, 1H), 1.26-1.12 (m, 1H).

### Example 2: Synthesis of Compound 2

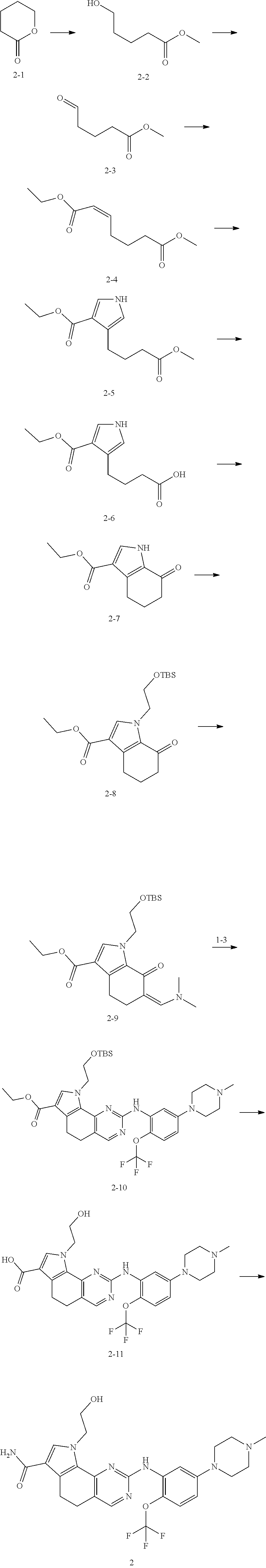

### 2.1 Synthesis of Compound 2-2

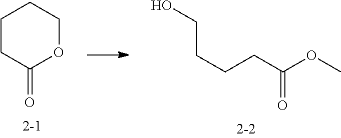

Intermediate 2-1 (5.00 g, 49.9 mmol) was dissolved in methanol (20 mL), concentrated sulfuric acid (0.33 mL) was added dropwise, and the mixture was reacted at 65° C. for 48 h under nitrogen protection. Potassium carbonate (2.00 g) was added and the mixture was stirred at room temperature for 0.5 h. The reaction liquid was filtered, and the filtrate was collected and concentrated under reduced pressure. The residue was dissolved in water (50 mL), and extracted with ethyl acetate (50 mL×3). The organic phases were combined, dried over anhydrous sodium sulfate, filtered, and the organic phase was concentrated under reduced pressure. The intermediate 2-2 (5.48 g, crude product) was obtained, which was used directly in the next step. MS (ESI) m/z=133.2 [M+H]⁺.

### 2.2 Synthesis of Intermediate 2-3

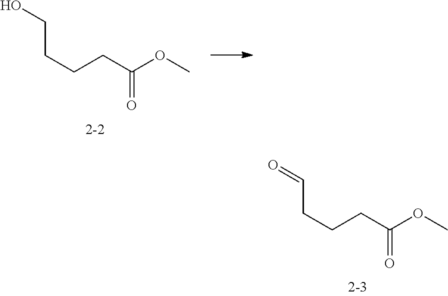

Intermediate 2-2 (5.85 g, 44.3 mmol), TEMPO (0.35 g, 2.20 mmol) and KBr (0.53 g, 4.40 mmol) were dissolved in dichloromethane (60 mL), NaClO (14.3 g, 23% aqueous solution) was added at −5° C., and the mixture was reacted at 0° C. for 1 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. Intermediate 2-3 (5.46 g, crude product, oil) was obtained and directly used in the next step.

### 2.3 Synthesis of Intermediate 2-4

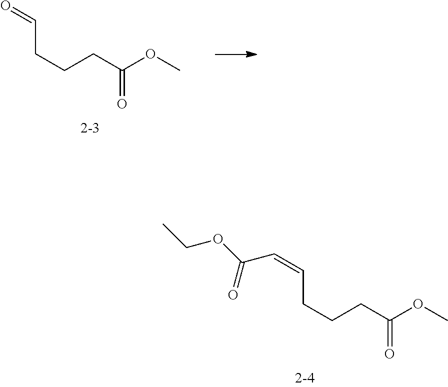

Intermediate 2-3 (5.46 g, 42.0 mmol) and ethyl (triphenylphosphaalkene)acetate (14.9 g, 42.8 mmol) were dissolved in toluene (50 mL) and the mixture was reacted at 120° C. for 12 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-15%) to obtain intermediate 2-4 (2.18 g, yield 21.7%, oil). MS (ESI) m/z=201.2 [M+H]⁺.

### 2.4 Synthesis of Intermediate 2-5

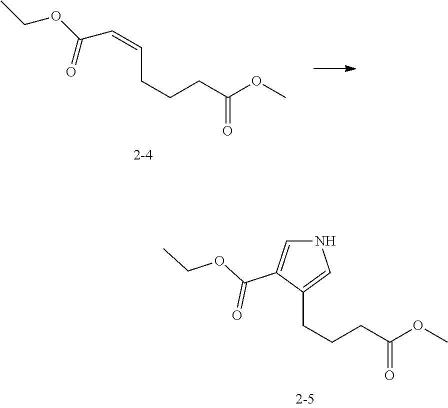

Under nitrogen protection and −78° C., a solution of 1 M LiHMDS in tetrahydrofuran (10.8 mL, 10.8 mmol) was added dropwise to 30 mL of a solution of TosMIC (2.11 g, 10.8 mmol) in tetrahydrofuran. The obtained product was stirred for 40 min at −78° C., a solution of intermediate 2-4 (1.10 g, 5.90 mmol) in tetrahydrofuran (30 mL) was added dropwise, and the mixture was reacted under stirring for 20 min and then brought to room temperature for reaction. After the reaction was completed by detection, water (100 mL) was added to quench the reaction, and dichloromethane (100 mL×3) was added for extraction. The organic phases were combined, dried over anhydrous sodium sulfate, filtered, and the organic phase was concentrated under reduced pressure. The residue was subjected to silica gel column chromatography (ethyl acetate:petroleum ether=30%) to obtain intermediate 2-5 (1.82 g, yield 70.6%, oil). MS (ESI) m/z=240.0 [M+H]⁺.

### 2.5 Synthesis of Intermediate 2-6

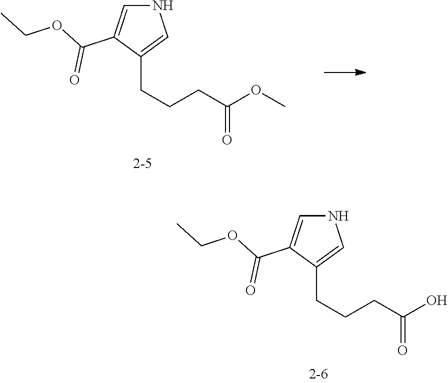

Intermediate 2-5 (1.00 g, 4.20 mmol) was dissolved in dioxane (20 mL) and water (5 mL), lithium hydroxide (0.100 g, 0.420 mmol) was added, and the mixture was reacted at room temperature for 12 h. The pH was adjusted to acidic with 1 N HCl, water (50 mL) was added, and ethyl acetate (50 mL×3) was added for extraction. The organic phases were combined, dried over anhydrous sodium sulfate, filtered, and the organic phase was concentrated under reduced pressure. The intermediate 2-6 (0.974 g, crude product) was obtained, which was used directly in the next step. MS (ESI) m/z=226.0 [M+H]⁺.

### 2.6 Synthesis of Intermediate 2-7

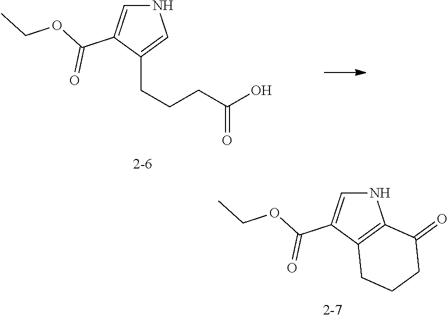 [SMILES: CCOC(=O)c1c[nH]cc1CCCC(=O)O ; CCOC(=O)c1c[nH]c2c1CCCC2=O]

Intermediate 2-6 (0.974 g, 4.32 mmol) was dissolved in trifluoroacetic acid (5 mL), TFAA (0.908 g, 4.32 mmol) was added, and the mixture was reacted at room temperature for 1 h. The reaction liquid was concentrated under reduced pressure. The residue was subjected to silica gel column chromatography (ethyl acetate/petroleum ether=0%-32%) to obtain intermediate 2-7 (0.46 g, yield 50.8%, oil). MS (ESI) m/z=208.0 [M+H]⁺.

### 2.7 Preparation of Intermediate 2-8

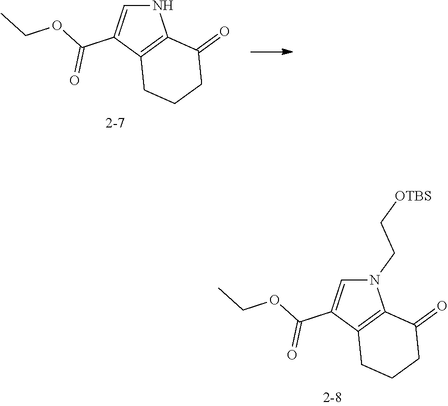 [SMILES: CCOC(=O)c1c[nH]c2c1CCCC2=O ; CCOC(=O)c1cn(CCO[Si](C)(C)C(C)(C)C)c2c1CCCC2=O]

Intermediate 2-7 (0.44 g, 2.12 mmol) and (2-bromoethoxy)-tert-butyldimethylsilane (0.61 g, 2.55 mmol) were dissolved in DMF (5 mL), K₂CO₃ (0.587 g, 4.24 mmol) was added, and the mixture was reacted at room temperature for 12 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-20%) to obtain intermediate 2-8 (0.64 g, yield 83.7%, oil). MS (ESI) m/z=366.2 [M+H]⁺.

### 2.8 Synthesis of Intermediate 2-9

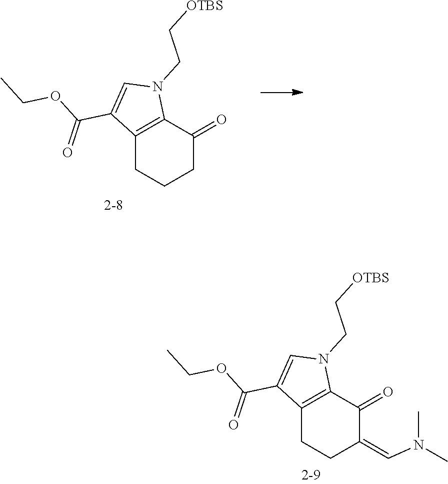 [SMILES: CCOC(=O)c1cn(CCO[Si](C)(C)C(C)(C)C)c2c1CCCC2=O ; CCOC(=O)c1cn(CCO[Si](C)(C)C(C)(C)C)c2c1CC/C(=C/N(C)C)C2=O]

Intermediate 2-8 (0.5 g, 1.37 mmol) was dissolved in DMF (5 mL), tert-butoxy bis(dimethylamino)methane (953 mg, 5.47 mmol) was added, and the mixture was reacted under microwave at 150° C. for 4 h. The mixture was cooled to room temperature, water (50 mL) was added to quench the reaction, and ethyl acetate (50 mL×3) was added for extraction. The organic phases were combined, dried over anhydrous sodium sulfate, filtered, and the organic phase was concentrated under reduced pressure. The intermediate 2-9 (0.6 g, crude product) was obtained, which was used directly in the next step.

### 2.9 Synthesis of Intermediate 2-10

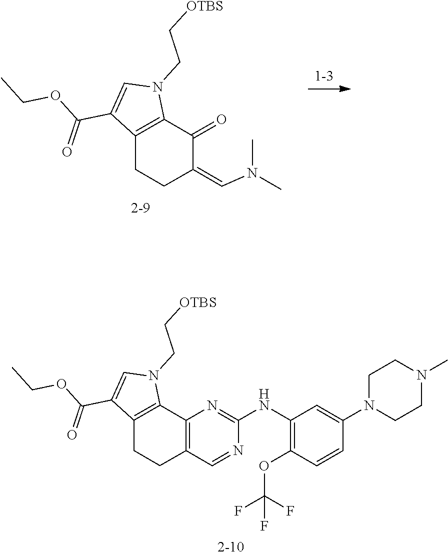

Intermediates 2-9 (0.45 g, 1.07 mmol) and 1-3 (0.34 g, 1.07 mmol) were dissolved in DMF (10 mL). The mixture was reacted at 120° C. for 12 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (methanol/dichloromethane=0%-6%) to obtain intermediate 2-10 (0.30 g, yield 42.1%, brown solid). MS (ESI) m/z=675.4 [M+H]⁺.

### 2.10 Synthesis of Intermediate 2-11

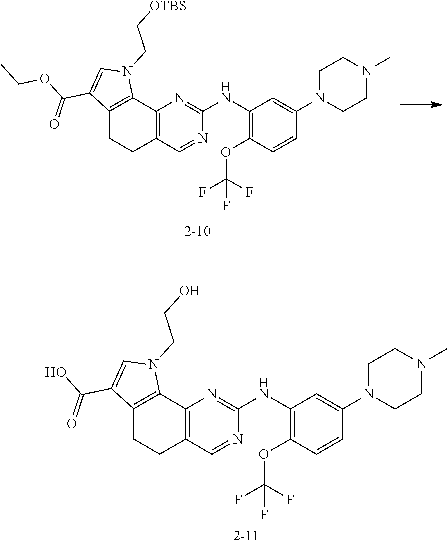 [SMILES: CCOC(=O)c1cn(CCO[Si](C)(C)C(C)(C)C)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)O)cn3CCO)CC4)c2)CC1]

Intermediate 2-10 (0.289 g, 0.428 mmol) and LiOH (0.103 g, 4.28 mmol) were dissolved in methanol (7 mL) and water (3 mL). The mixture was reacted at 50° C. for 24 h. The reaction liquid was adjusted to acidic pH with dilute hydrochloric acid and concentrated under reduced pressure. The residue was subjected to column chromatography (C18, acetonitrile/water=0%-20%) to obtain intermediate 2-11 (0.16 g, yield 72.7%, pink solid). MS (ESI) m/z=533.1[M+H]⁺.

### 2.11 Synthesis of Compound 2

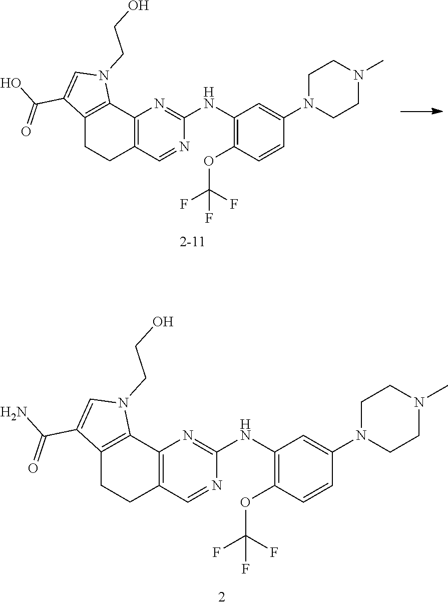 [SMILES: CN1CCN(c2ccc(OC(F)(F)F)c(Nc3ncc4c(n3)-c3c(c(C(=O)O)cn3CCO)CC4)c2)CC1 ; CN1CCN(c2ccc(OC(F)(F)F)c(Nc3ncc4c(n3)-c3c(c(C(N)=O)cn3CCO)CC4)c2)CC1]

Intermediate 2-11 (0.30 g, 0.563 mmol), ammonium chloride (0.302 g, 5.63 mmol), HOBt (0.0989 g, 0.732 mmol) and DIEA (0.291 g, 2.25 mmol) were dissolved in dichloromethane (10 mL), and EDCI (0.14 g, 0.732 mmol) was added. The mixture was reacted at 50° C. for 12 h. The reaction liquid was concentrated under reduced pressure. The residue was subjected to column chromatography (C18, acetonitrile/water (0.1% FA)=38%) to obtain compound 2 (0.10 g, yield 34.2%, yellow solid). MS (ESI) m/z=532.2 [M+H]⁺. ¹HNMR (400 MHz, DMSO-d₆) δ 8.52 (s, 1H), 8.11 (s, 1H), 7.52 (s, 1H), 7.36-7.14 (m, 3H), 6.94-6.66 (m, 2H), 4.68 (t, J=4.8 Hz, 1H), 4.32 (t, J=4.8 Hz, 2H), 3.42-3.37 (m, 2H), 3.17-3.09 (m, 4H), 2.99-2.92 (m, 2H), 2.73-2.66 (m, 2H), 2.46-2.41 (m, 4H), 2.22 (s, 3H).

### Example 3: Synthesis of Compound 3

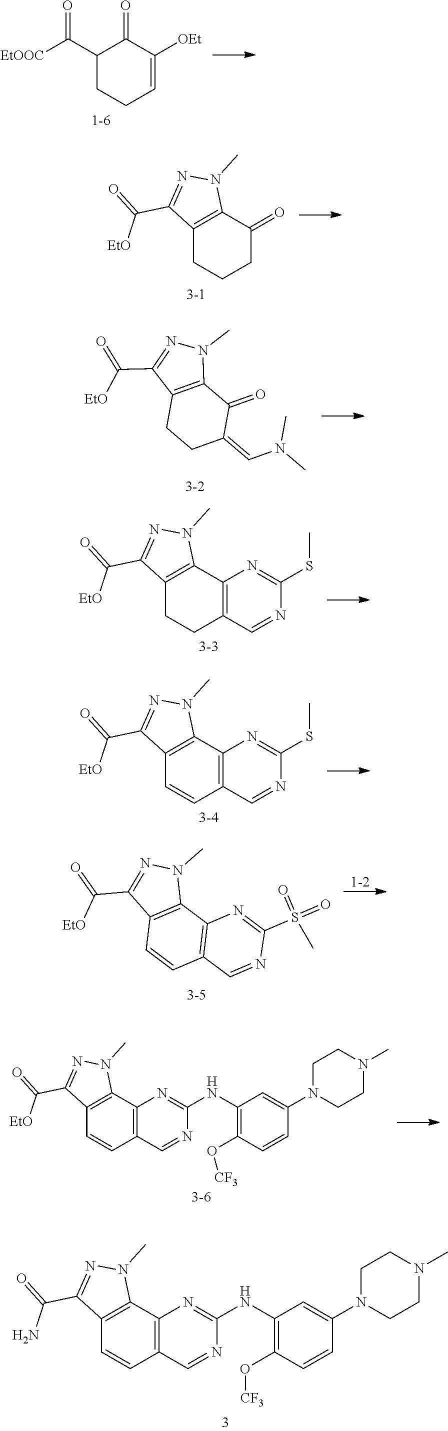

### 3.1 Synthesis of Intermediate 3-1

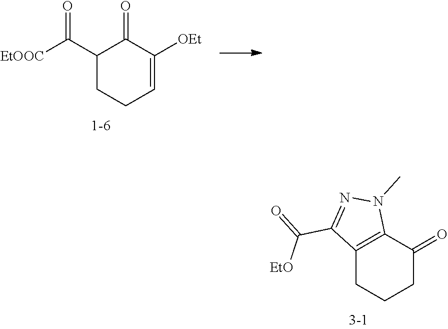 [SMILES: CCOC(=O)C(=O)C1CCC=C(OCC)C1=O ; CCOC(=O)c1nn(C)c2c1CCCC2=O]

Intermediate 1-6 (20 g, 83.2 mmol) was dissolved in acetic acid (100 mL), methylhydrazine (6.2 g, 53.8 mmol) was added at 0° C., and the mixture was reacted at room temperature for 2 h. The reaction liquid was concentrated under reduced pressure, and the residue was dissolved in water (500 mL) and adjusted to pH 7-8 with saturated NaHCO₃. Dichloromethane (100 mL×3) was added for extraction, the organic phases were combined, dried over anhydrous sodium sulfate, and filtered, the organic phase was concentrated under reduced pressure, and the residue was subjected to silica gel column chromatography (dichloromethane 100%) to obtain the intermediate 3-1 (13.0 g, yield 70.3%, off-white solid). MS (ESI) m/z=223.2 [M+H]⁺.

### 3.2 Synthesis of Intermediate 3-2

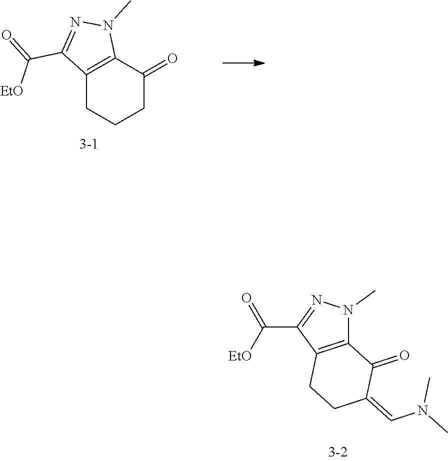 [SMILES: CCOC(=O)c1nn(C)c2c1CCCC2=O ; CCOC(=O)c1nn(C)c2c1CC/C(=C/N(C)C)C2=O]

Intermediate 3-1 (1 g, 4.50 mmol) was dissolved in DMF (5 mL), tert-butoxy bis(dimethylamino)methane (1.83 g, 9.00 mmol) was added, and the mixture was reacted at 80° C. for 4 h. The reaction liquid was concentrated under reduced pressure to obtain intermediate 3-2 (1.2 g, crude product), which was directly used in the next step.

### 3.3 Synthesis of Intermediate 3-3

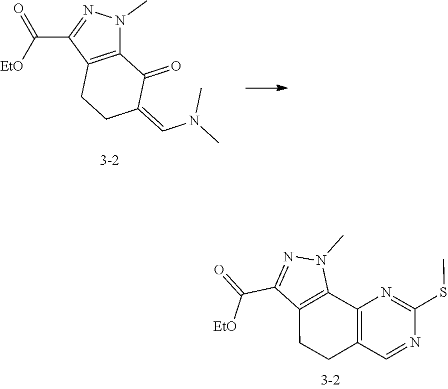 [SMILES: CCOC(=O)c1nn(C)c2c1CC/C(=C/N(C)C)C2=O ; CCOC(=O)c1nn(C)c2c1CCc1cnc(SC)nc1-2]

Intermediate 3-2 (5 g, 18.0 mmol) was dissolved in DMF (50 mL), 2-methyl-2-mercaptourea sulfate (3.01 g, 10.8 mmol) and potassium acetate (1.77 g, 18.0 mmol) were added, and the mixture was reacted 95° C. for 4 h. Water (200 mL) was added to quench the reaction and the solid was precipitated, filtered and collected. The solid was purified by silica gel column chromatography (petroleum ether:ethyl acetate=2:1) to obtain intermediate 3-3 (2.76 g, yield 50.5%, yellow solid). MS (ESI) m/z=305.1 [M+H]⁺.

### 3.4 Synthesis of Intermediate 3-4

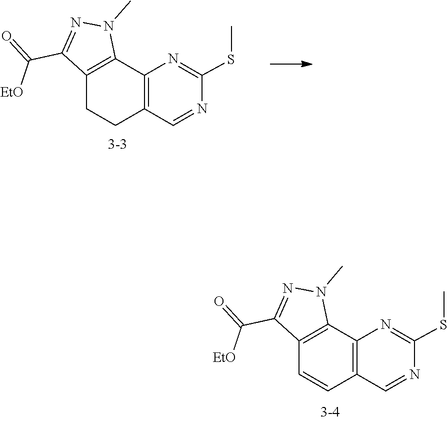 [SMILES: CCOC(=O)c1nn(C)c2c1CCc1cnc(SC)nc1-2 ; CCOC(=O)c1nn(C)c2c1ccc1cnc(SC)nc12]

Intermediate 3-3 (1.32 g, 4.34 mmol) was dissolved in chlorobenzene (50 mL), and DDQ (1.97 g, 8.67 mmol) was added. The mixture was reacted at 135° C. for 16 h. The reaction liquid was concentrated under reduced pressure, and the residue was purified by silica gel column chromatography (dichloromethane) to obtain intermediate 3-4 (0.76 g, yield 57.8%, brown solid). MS (ESI) m/z=302.9 [M+H]⁺.

### 3.5 Synthesis of Intermediate 3-5

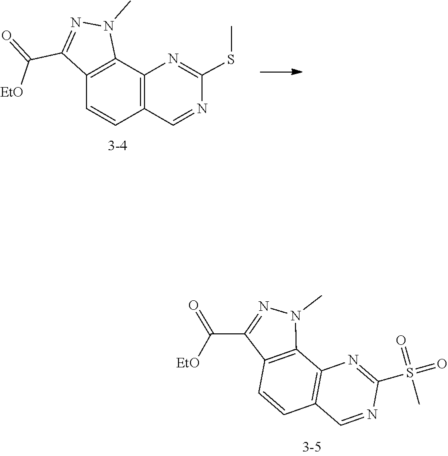 [SMILES: CCOC(=O)c1nn(C)c2c1ccc1cnc(SC)nc12 ; CCOC(=O)c1nn(C)c2c1ccc1cnc(S(C)(=O)=O)nc12]

Intermediate 3-4 (0.86 g, 2.84 mmol) was dissolved in DMF (30 mL), potassium peroxymonosulfonate (5.25 g, 8.53 mmol) was added, and the mixture was reacted at room temperature for 16 h. Ice water was added to quench the reaction, the solid was precipitated, and filtered. The filter cake was dissolved in tetrahydrofuran (30 mL), and the solution was concentrated under reduced pressure. These operations were repeated three times. Intermediate 3-5 (0.80 g, crude product, white solid) was obtained, which was used directly in the next step. MS (ESI) m/z=335.1 [M+H]⁺.

### 3.6 Synthesis of Intermediate 3-6

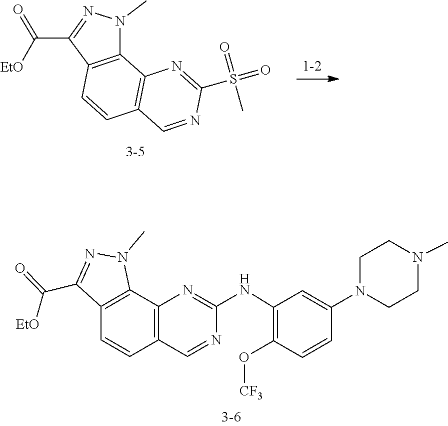 [SMILES: CCOC(=O)c1nn(C)c2c1ccc1cnc(S(C)(=O)=O)nc12 ; CCOC(=O)c1nn(C)c2c1ccc1cnc(Nc3cc(N4CCN(C)CC4)ccc3OC(F)(F)F)nc12]

Intermediate 1-2 (0.284 g, 1.03 mmol) was dissolved in tetrahydrofuran (10 mL), LiHMDS (1 M, 1.55 mL) was added, and the mixture was reacted under stirring at −78° C. for 0.5 h. Intermediate 3-5 (0.23 g, 0.688 mmol) was added and the mixture was reacted at room temperature for 12 h. Water (100 mL) was added to quench the reaction, dichloromethane (50 mL×3) was added for extraction, the organic phases were combined, dried over anhydrous sodium sulfate, and filtered, the organic phase was concentrated under reduced pressure, and the residue was subjected to column chromatograph (C18 150*40 mm*10 μm; mobile phase: [H₂O(NH₃H₂O+NH₄HCO₃)-ACN]; B %: 50%-90%, 10 min) to obtain intermediate 3-6 (0.11 g, yield 20.5%, yellow solid). MS (ESI) m/z=530.3 [M+H]⁺.

### 3.7 Synthesis of Compound 3

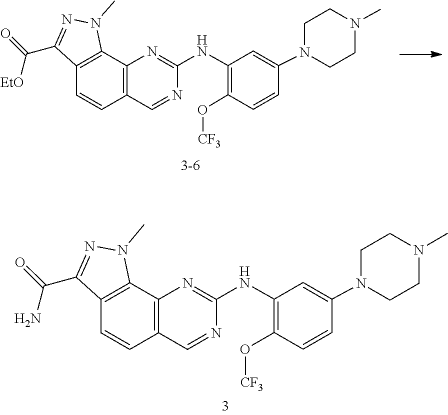 [SMILES: CCOC(=O)c1nn(C)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(N)=O)nn(C)c5c4n3)c2)CC1]

Compound 3-6 (40 mg, 75.5 umol) was dissolved in DMF (10 mL) and a solution of ammonia in methanol (75 mL) was added. The mixture was reacted for 48 h under sealed conditions at 135° C. The reaction liquid was concentrated under reduced pressure, and the residue was subjected to column chromatography (C18 75*30 mm*3 μm; mobile phase: [H₂O(NH₄HCO₃)-ACN]; B %: 30%-90%, 28 min). Compound 3 (3.9 mg, yield 10.4%, white solid) was obtained. MS (ESI) m/z=501.1 [M+H]⁺. ¹H NMR (400 MHz, CD₃OD) δ 9.03-9.09 (m, 1H). 7.98-8.08 (m, 2H) 7.49-7.54 (m, 1H) 7.19-7.27 (m, 1H) 6.76-6.82 (m, 1H) 4.71-4.75 (m, 3H) 4.53-4.57 (m, 4H) 4.30 (br, 1H) 3.29 (br, 4H) 2.59-2.70 (m, 4H) 2.31-2.40 (m, 3H).

### Example 4: Synthesis of Compound 4

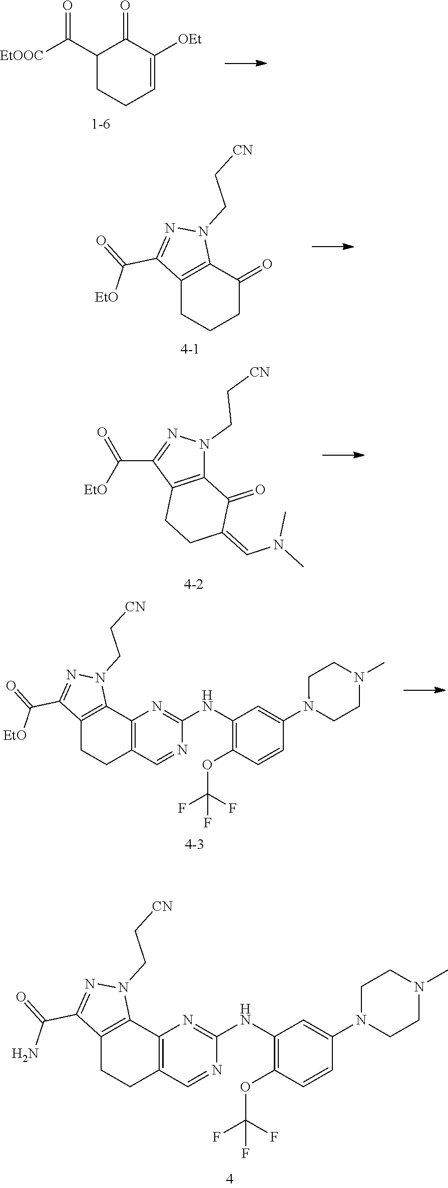 [SMILES: CCOC(=O)C(=O)C1CCC=C(OCC)C1=O ; CCOC(=O)c1nn(CCC#N)c2c1CCCC2=O ; CCOC(=O)c1nn(CCC#N)c2c1CC/C(=C/N(C)C)C2=O ; CCOC(=O)c1nn(CCC#N)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(N)=O)nn3CCC#N)CC4)c2)CC1]

### 4.1 Synthesis of Intermediate 4-1

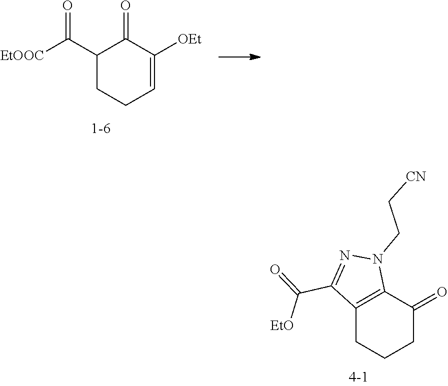 [SMILES: CCOC(=O)C(=O)C1CCC=C(OCC)C1=O ; CCOC(=O)c1nn(CCC#N)c2c1CCCC2=O]

Intermediate 1-6 (10.0 g, 41.6 mmol) was dissolved in acetic acid, 2-cyanoethylhydrazine (4.25 g, 50.0 mmol) was added, and the mixture was reacted at 25° C. for 6 h. The reaction liquid was concentrated under reduced pressure, and the residue was purified by silica gel column chromatography (ethyl acetate:petroleum ether=0-10%) to obtain intermediate 4-1 (5.4 g, yield 49.6%, white solid). MS (ESI) m/z=261.9 [M+H]⁺.

### 4.2 Synthesis of Intermediate 4-2

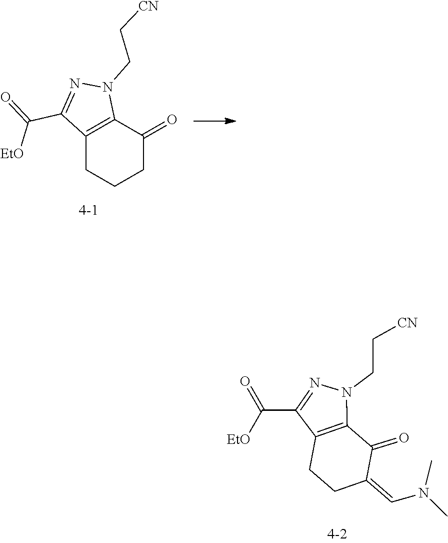 [SMILES: CCOC(=O)c1nn(CCC#N)c2c1CCCC2=O ; CCOC(=O)c1nn(CCC#N)c2c1CC/C(=C/N(C)C)C2=O]

Intermediate 4-1 (0.50 g, 1.91 mmol) was dissolved in DMF (10 mL), tert-butoxy bis(dimethylamino)methane (0.95 g, 3.83 mmol) was added, and the mixture was reacted at 110° C. for 16 h. The reaction liquid was concentrated under reduced pressure, and the residue was dissolved in methyl tert-butyl ether (50 mL) and the mixture was stirred at 25° C. for 0.5 h to precipitate a solid. After suction filtration, the filter cake was dried to obtain intermediate 4-2 (1.2 g, crude product), which was directly used in the next step.

### 4.3 Synthesis of Intermediate 4-3

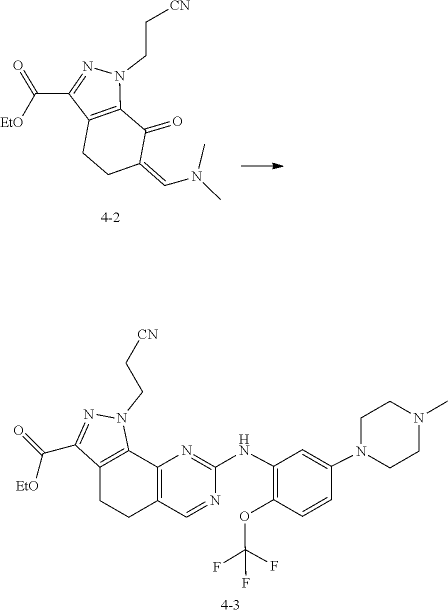 [SMILES: CCOC(=O)c1nn(CCC#N)c2c1CC/C(=C/N(C)C)C2=O ; CCOC(=O)c1nn(CCC#N)c2c1CCc1cnc(Nc3cc(N4CCN(C)CC4)ccc3OC(F)(F)F)nc1-2]

Intermediate 4-2 (1.2 g, 1.91 mmol) was dissolved in DMF (10 mL), intermediate 1-3 (0.90 g, 2.84 mmol) was added, and the mixture was reacted at 110° C. for 6 h. Water (200 mL) was added to quench the reaction, ethyl acetate (200 mL×3) was added for extraction, the organic phases were combined, dried over anhydrous sodium sulfate, and filtered, the organic phase was concentrated under reduced pressure, and the residue was subjected to silica gel column chromatograph (methanol:dichloromethane=0-10%). The intermediate 4-3 (0.47 g, yield 43.5%, brown solid) was obtained. MS (ESI) m/z=571.3 [M+H]⁺.

### 4.4 Synthesis of Compound 4

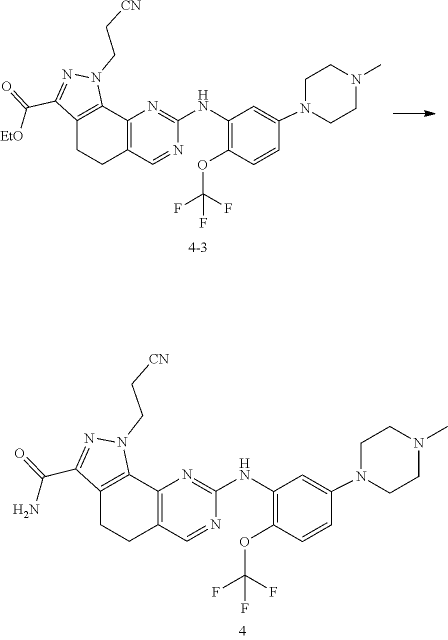 [SMILES: CCOC(=O)c1nn(CCC#N)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(N)=O)nn3CCC#N)CC4)c2)CC1]

Intermediate 4-3 (0.10 g, 0.175 mmol) was dissolved in methanol (5 mL), ammonia water (2 mL) was added, and the mixture was reacted under sealed conditions at 85° C. for 48 h. The reaction liquid was concentrated under reduced pressure, and the residue was dissolved in ethyl acetate (50 mL) and the obtained product was washed with water (50 mL×2). The organic phase was collected, dried over anhydrous sodium sulfate, filtered, and concentrated under reduced pressure. The residue was subjected to column chromatography (C18 75*30 mm*3 μm; mobile phase: [H₂O (0.05% NH₃H₂O 10 mM NH₄HCO₃)-ACN]; B %: 29%-59%, 11 min). Compound 4 (0.02 g, yield 20.6%, white solid) was obtained. MS (ESI) m/z=542.3 [M+H]⁺. ¹H NMR (400 MHz, DMSO-d6) δ 9.03-9.06 (m, 1H). 9.00-9.07 (m, 1H). 8.33-8.38 (m, 1H). 7.43-7.50 (m, 1H). 7.33-7.38 (m, 1H). 7.18-7.28 (m, 2H). 6.75-6.83 (m, 1H). 4.72-4.81 (m, 2H). 3.11-3.18 (m, 4H). 2.94-3.02 (m, 2H). 2.91 (t, J=6.40 Hz, 2H) 2.76-2.83 (m, 2H). 2.29 (br, 2H). 2.25-2.39 (m, 1H). 2.17-2.24 (m, 3H).

### Example 5: Synthesis of Compound 5

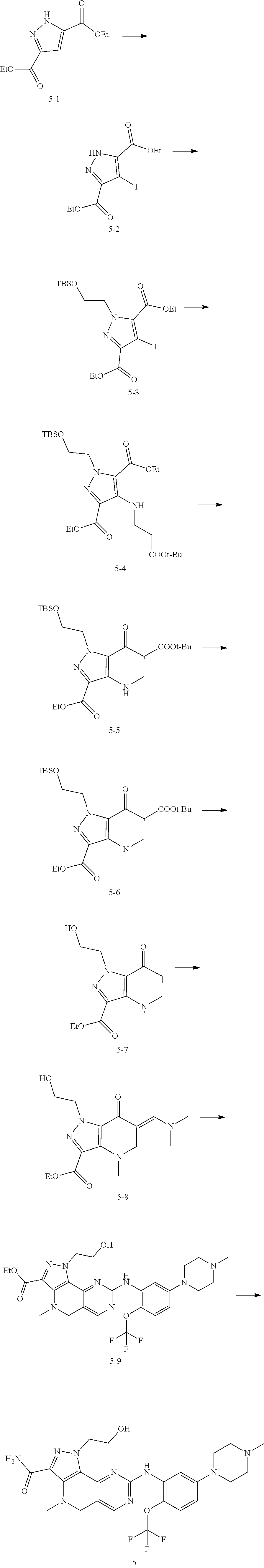

### 5.1 Synthesis of Intermediate 5-2

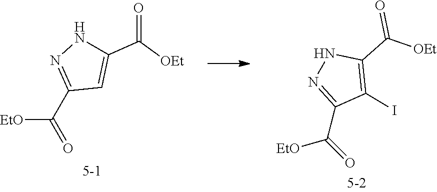 [SMILES: CCOC(=O)c1cc(C(=O)OCC)[nH]n1 ; CCOC(=O)c1n[nH]c(C(=O)OCC)c1I]

Intermediate 5-1 (30.0 g, 141 mmol) was dissolved in acetonitrile (500 mL), CAN (38.8 g, 70.7 mmol) and iodine (21.5 g, 84.8 mmol) were added, and the mixture was reacted at 80° C. for 16 h. Sodium thiosulfate saturated solution (500 mL) was added to quench the reaction, and ethyl acetate (300 mL×3) was added for extraction. The organic phases were combined, dried over anhydrous sodium sulfate, filtered, and the organic phase was concentrated under reduced pressure. The residue was subjected to silica gel column chromatography (ethyl acetate:petroleum ether=0-30%) to obtain intermediate 5-2 (37.4 g, yield 78.3%, yellow solid). MS (ESI) m/z=339.0 [M+H]⁺.

### 5.2 Synthesis of Intermediate 5-3

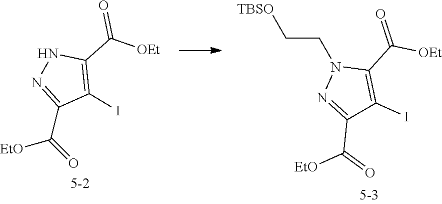 [SMILES: CCOC(=O)c1n[nH]c(C(=O)OCC)c1I ; CCOC(=O)c1nn(CCO[Si](C)(C)C(C)(C)C)c(C(=O)OCC)c1I]

Intermediate 5-2 (40.0 g, 118 mmol) was dissolved in acetonitrile (400 mL), K₂CO₃ (24.5 g, 177 mmol) and (2-bromoethoxy)-tert-butyldimethylsilane (34.0 g, 142 mmol) were added and the mixture was reacted at 80° C. for 16 h. The reaction liquid was filtered, the filtrate was concentrated under reduced pressure, and the residue was subjected to silica gel column chromatography (ethyl acetate:petroleum ether=0-20%). The intermediate 5-3 (54.4 g, yield 92.9%, white solid) was obtained. MS (ESI) m/z=497.1 [M+H]V.

### 5.3 Synthesis of Intermediate 5-4

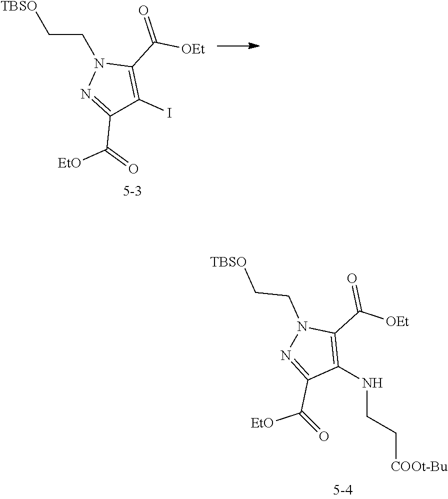

Intermediate 5-3 (50.0 g, 101 mmol), tert-butyl 3-aminopropionate hydrochloride (27.5 g, 151 mmol), cesium carbonate (131 g, 403 mmol) and 4,5-bisdiphenylphosphine-9,9-dimethylxanthene (2.91 g, 5.04 mmol) was dissolved in dioxane (500 mL), and Pd₂(dba)₃ (4.61 g, 5.04 mmol) was added, the mixture was reacted under nitrogen protection at 110° C. for 16 h. Ammonium chloride saturated solution (500 mL) was added to quench the reaction, ethyl acetate (500 mL×3) was added for extraction, the organic phases were combined, dried over anhydrous sodium sulfate, and filtered, and the organic phase was concentrated under reduced pressure. The residue was subjected to silica gel column chromatography (ethyl acetate:petroleum ether=0-20%) to obtain intermediate 5-4 (38.9 g, yield 75.0%, oil). MS (ESI) m/z=514.3 [M+H]⁺.

### 5.4 Synthesis of Intermediate 5-5

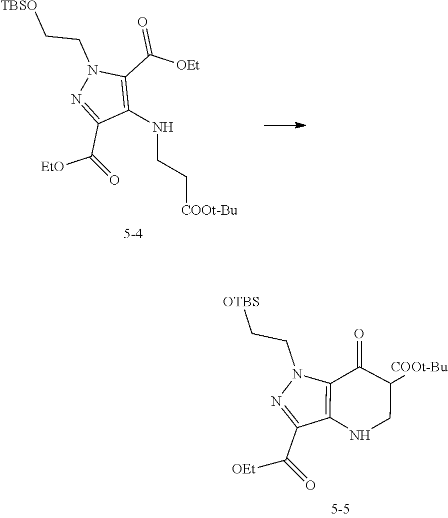

Intermediate 5-4 (24.0 g, 46.7 mmol) was dissolved in tetrahydrofuran (300 mL), potassium tert-butoxide (10.5 g, 93.4 mmol) was added, and the mixture was stirred at −20° C. for 2 h. Ammonium chloride saturated solution (300 mL) was added to quench the reaction, ethyl acetate (300 mL×3) was added for extraction, the organic phases were combined, dried over anhydrous sodium sulfate, and filtered, and the organic phase was concentrated under reduced pressure. The residue was subjected to silica gel column chromatography (ethyl acetate:petroleum ether=0-30%) to obtain intermediate 5-5 (8.9 g, yield 40.6%, yellow solid). MS (ESI) m/z=468.2 [M+H]⁺.

### 5.5 Synthesis of Intermediate 5-6

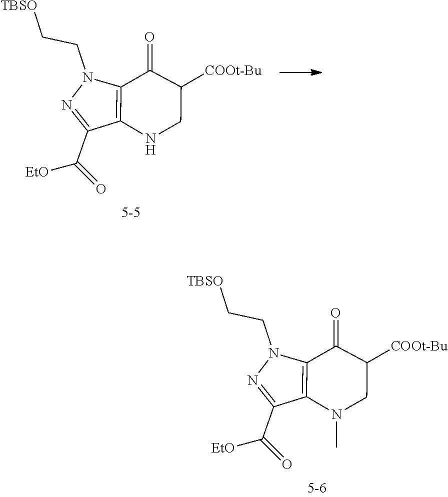

Intermediate 5-5 (5.00 g, 10.7 mmol) was dissolved in acetic acid (50 mL), formaldehyde solution (2.60 g, 32.1 mmol) and NaBH₃CN (1.34 g, 21.4 mmol) were added at 0° C. The mixture was reacted at room temperature for 2 h. Na₂CO₃ saturated solution (10 mL) was added to quench the reaction, ethyl acetate (10 mL×2) was added for extraction, the organic phases were combined, dried over anhydrous sodium sulfate, and filtered, and the organic phase was concentrated under reduced pressure. The residue was subjected to silica gel column chromatography (ethyl acetate:petroleum ether=30%) to obtain intermediate 5-6 (3.50 g, yield 68.0%, yellow solid). MS (ESI) m/z=482.3 [M+H]⁺.

### 5.6 Synthesis of Intermediate 5-7

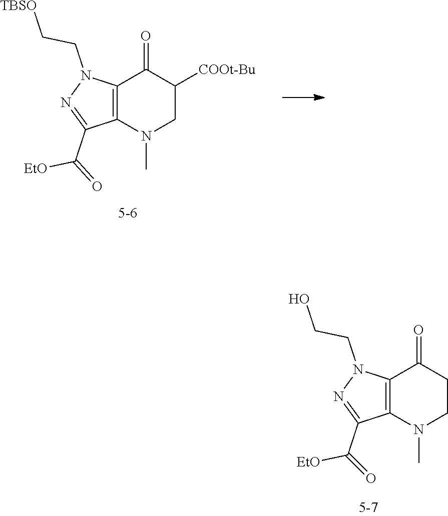

Intermediate 5-6 (1.00 g, 2.08 mmol) was dissolved in ethanol (5 mL) and hydrochloric acid (3 M, 15.0 mL). The mixture was reacted at 60° C. for 16 h. Sodium carbonate saturated solution (50 mL) was added to quench the reaction, ethyl acetate (50 mL×3) was added for extraction, the organic phases were combined, dried over anhydrous sodium sulfate, and filtered, and the organic phase was concentrated under reduced pressure. The residue was subjected to silica gel column chromatography (ethyl acetate:petroleum ether=0-100%) to obtain intermediate 5-7 (0.35 g, yield 63.1%, white solid). MS (ESI) m/z=268.1 [M+H]⁺.

### 5.7 Synthesis of Intermediate 5-8

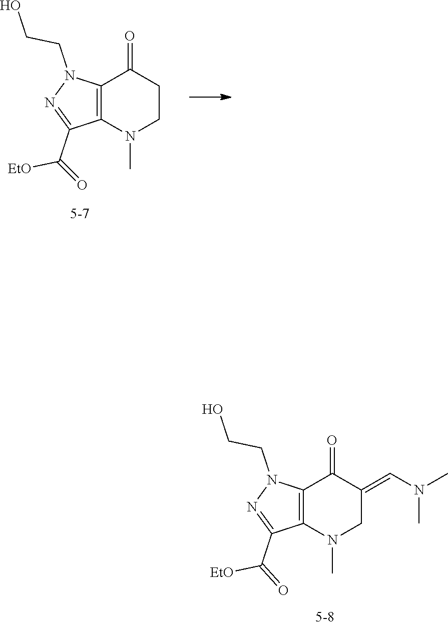 [SMILES: CCOC(=O)c1nn(CCO)c2c1N(C)CCC2=O ; CCOC(=O)c1nn(CCO)c2c1N(C)C/C(=C\N(C)C)C2=O]

Intermediate 5-7 (0.30 g, 1.12 mmol) was dissolved in DMF (3 mL), tert-butoxy bis(dimethylamino)methane (0.456 g, 2.24 mmol) was added. The mixture was reacted at 60° C. for 16 h. The reaction liquid was concentrated under reduced pressure to obtain intermediate 5-8 (0.25 g, crude product), which was directly used in the next step.

### 5.8 Synthesis of Intermediate 5-9

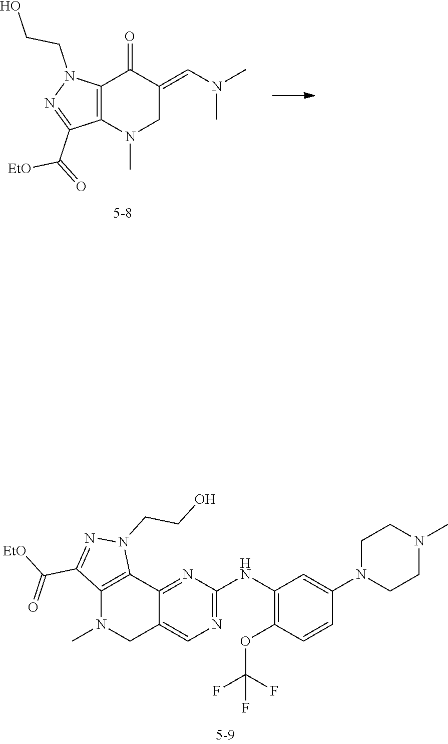 [SMILES: CCOC(=O)c1nn(CCO)c2c1N(C)C/C(=C\N(C)C)C2=O ; CCOC(=O)c1nn(CCO)c2c1N(C)Cc1cnc(Nc3cc(N4CCN(C)CC4)ccc3OC(F)(F)F)nc1-2]

Intermediate 5-8 (0.10 g, 0.31 mmol), intermediate 1-3 (0.13 g, 0.31 mmol) and sodium ethoxide (0.025 g, 0.37 mmol) were dissolved in ethanol (10 mL). The mixture was reacted under microwave at 120° C. for 1.5 h. The reaction liquid was concentrated under reduced pressure, and the residue was subjected to column chromatography (C18 150*40 mm*10 μm; mobile phase: [H₂O (10 mM NH₄HCO₃)-ACN]; B %: 20%-50%, 8 min), and the intermediate 5-9 (0.0034 g, yield 1.88%) was obtained. MS (ESI) m/z=577.2 [M+H]⁺.

### 5.9 Synthesis of Compound 5

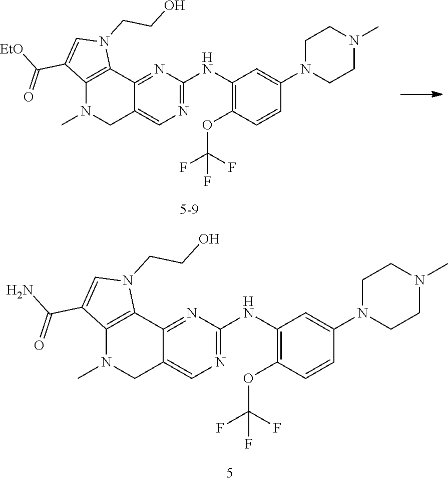 [SMILES: CCOC(=O)c1cn(CCO)c2c1N(C)Cc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(N)=O)cn3CCO)N(C)C4)c2)CC1]

Intermediate 5-9 (5.00 mg, 8.67 μmol) was dissolved in tetrahydrofuran (1 mL), NH₄Cl (1.45 mg, 27.1 mol) was added, and LiHMDS (1 M, 54.2 μL) was added at 0° C. The mixture was reacted at room temperature for 16 h. Water (2 mL) was added to quench the reaction, ethyl acetate (5 mL*3) was added for extraction, the organic phases were combined, dried over anhydrous sodium sulfate, and filtered, and the organic phase was concentrated under reduced pressure. The residue was subjected to column chromatography (C18 150*40 mm*10 μm; mobile phase: [H₂O (10 mM NH₄HCO₃)-ACN]; B %: 20%-50%, 8 min), and the compound 5 (1.61 mg, yield 32.4%) was obtained. MS (ESI) m/z=548.1 [M+H]⁺. ¹HNMR (400 MHz, DMSO-d₆) δ 8.88 (s, 1H), 8.24 (s, 1H), 7.42 (br s, 1H), 7.14-7.32 (m, 3H), 6.75-5.77 (m, 1H), 4.64 (br, 1H), 4.52 (br, 2H), 4.09 (s, 2H), 3.64 (br, 2H), 3.31 (br, 3H), 3.10-3.17 (m, 4H), 2.94 (s, 3H), 2.43 (br, 4H).

### Example 6: Synthesis of Compound 6

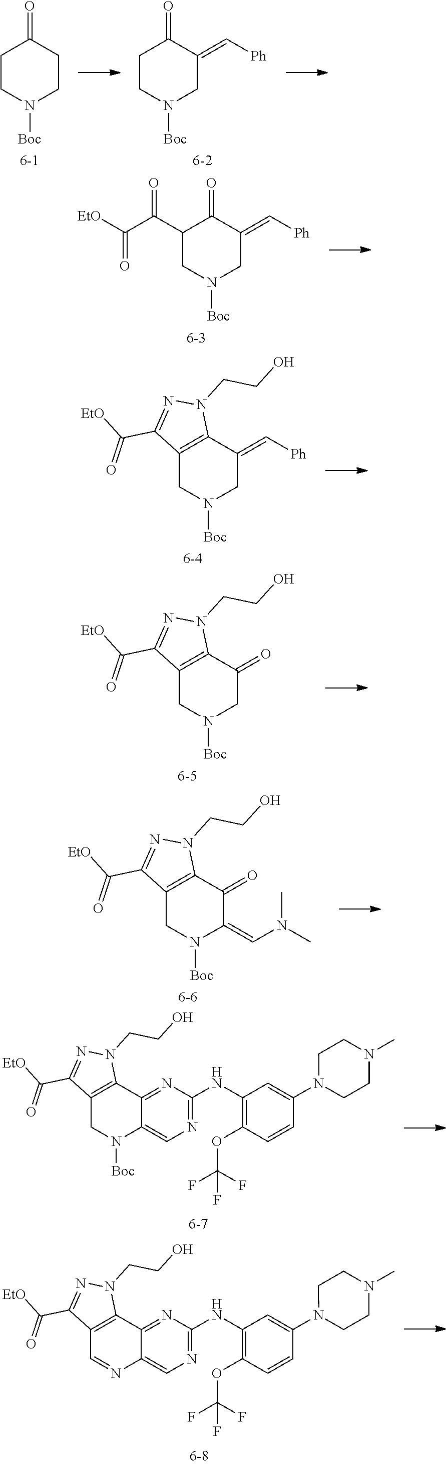

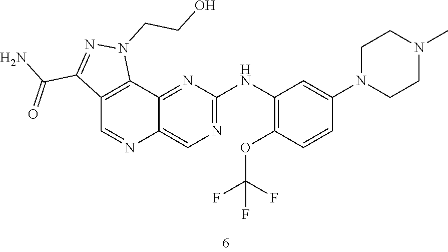 [SMILES: CN1CCN(c2ccc(OC(F)(F)F)c(Nc3ncc4ncc5c(C(N)=O)nn(CCO)c5c4n3)c2)CC1]

### 6.1 Synthesis of Intermediate 6-2

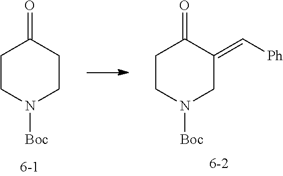

Intermediate 6-1 (8.0 g, 40.1 mmol) was dissolved in toluene (100 mL), and morpholine (5.8 g, 66.4 mmol), p-toluenesulfonic acid (0.034 g, 0.2 mmol) and benzaldehyde (4.0 mL, 40.0 mmol) were added and the mixture was reacted at 110° C. for 16 h. The reaction liquid was concentrated under reduced pressure, and the residue was subjected to silica gel column chromatography (ethyl acetate:petroleum ether=25%) to obtain intermediate 6-2 (2.9 g, yield 25%, yellow solid). MS (ESI) m/z=288.2 [M+H]⁺.

### 6.2 Synthesis of Intermediate 6-3

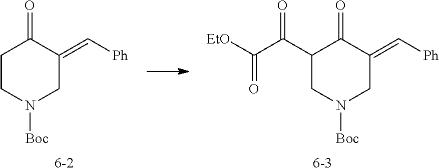

Intermediate 6-2 (2.87 g, 10 mmol) was dissolved in tetrahydrofuran (50 mL), LiHMDS (1 M, 12 mL) was added dropwise at −50.0° C., and the mixture was stirred for 30 min. Diethyl oxalate (1.75 g, 12 mmol) was added and reacted at room temperature for 16 h. Saturated ammonium chloride (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-20%) to obtain intermediate 6-3 (2.8 g, yield 73.7%, oil). MS (ESI) m/z=388.2 [M+H]⁺.

### 6.3 Synthesis of Intermediate 6-4

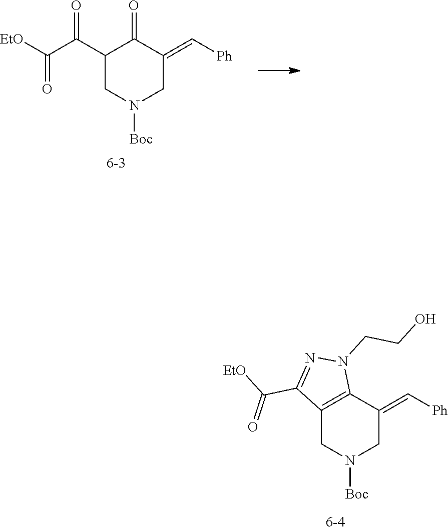

Intermediate 6-3 (2.85 g, 7.36 mmol) was dissolved in acetic acid (10 mL), hydroxyethylhydrazine (0.57 g, 7.57 mmol) was added, and the mixture was reacted at room temperature for 15 h. water (50 mL) was added to quench the reaction, saturated Na₂CO₃ (60 mL) was added, and dichloromethane (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-50%) to obtain intermediate 6-4 (1.59 g, yield 50.7%, yellow solid). MS (ESI) m/z=428.2 [M+H]⁺.

### 6.4 Synthesis of Intermediate 6-5

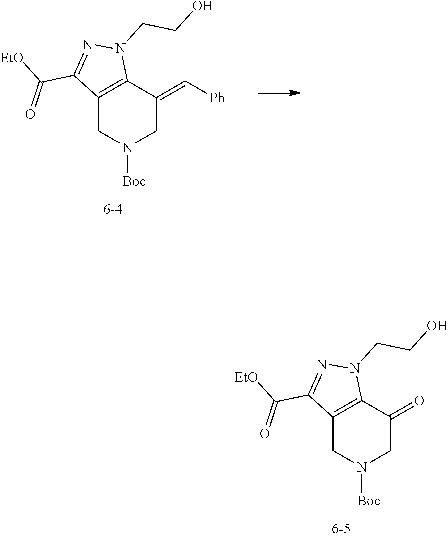 [SMILES: CCOC(=O)c1nn(CCO)c2c1CN(C(=O)OC(C)(C)C)C/C2=C\c1ccccc1 ; CCOC(=O)c1nn(CCO)c2c1CN(C(=O)OC(C)(C)C)CC2=O]

Intermediate 6-4 (1.58 g, 3.70 mmol) was dissolved in tetrahydrofuran (16 mL) and water (16 mL), and NaIO₄ (1.58 g, 7.40 mmol) and K₂OsO₄·2H₂O (0.11 g, 0.37 mmol) were added. The mixture was reacted at room temperature for 5 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-30%) to obtain intermediate 6-5 (0.313 g, yield 24%, yellow solid). MS (ESI) m/z=354.2 [M+H]⁺.

### 6.5 Synthesis of Intermediate 6-6

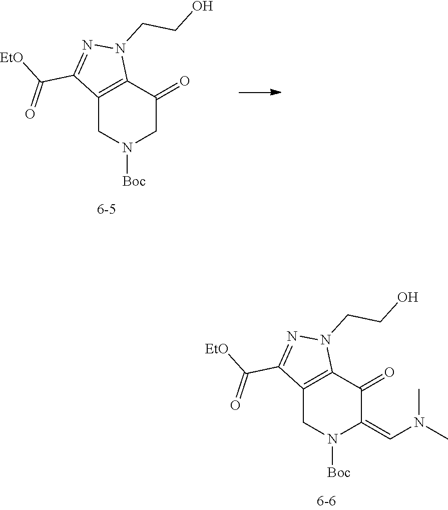 [SMILES: CCOC(=O)c1nn(CCO)c2c1CN(C(=O)OC(C)(C)C)CC2=O ; CCOC(=O)c1nn(CCO)c2c1CN(C(=O)OC(C)(C)C)/C(=C/N(C)C)C2=O]

Intermediate 6-5 (0.313 g, 0.89 mmol) was dissolved in DMF-DMA (10 mL) and the mixture was reacted at 110° C. for 12 h. The reaction liquid was concentrated under reduced pressure, and the residue was dissolved in methyl tertiary ether and the mixture was stirred for 3 h. The resulting product was filtered, the filter cake was collected, and dried to obtain intermediate 6-6 (0.36 g, crude product), which was directly used in the next step.

### 6.6 Synthesis of Intermediate 6-7

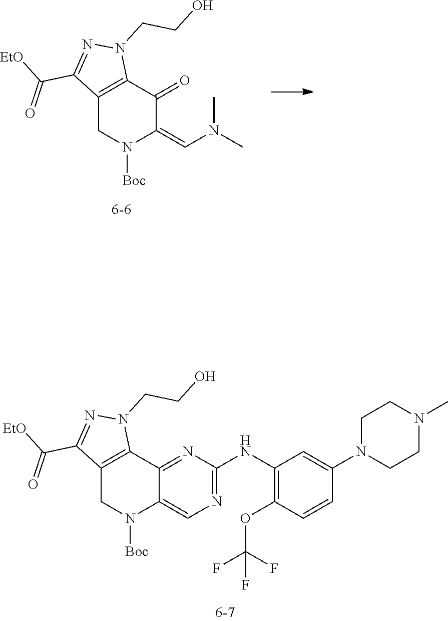 [SMILES: CCOC(=O)c1nn(CCO)c2c1CN(C(=O)OC(C)(C)C)/C(=C/N(C)C)C2=O ; CCOC(=O)c1nn(CCO)c2c1CN(C(=O)OC(C)(C)C)c1cnc(Nc3cc(N4CCN(C)CC4)ccc3OC(F)(F)F)nc1-2]

Intermediate 6-6 (0.16 g, 0.40 mmol) and intermediate 1-3 (0.14 g, 0.44 mmol) were dissolved in DMF (5 mL) and the mixture was reacted at 110° C. for 12 h. The reaction liquid was concentrated under reduced pressure, and the residue was subjected to silica gel column chromatography (ethyl acetate/petroleum ether=0%-100%) to obtain intermediate 6-7 (0.14 g, yield 53.3%, yellow solid). MS (ESI) m/z=663.2 [M+H]⁺.

### 6.7 Synthesis of Intermediate 6-8

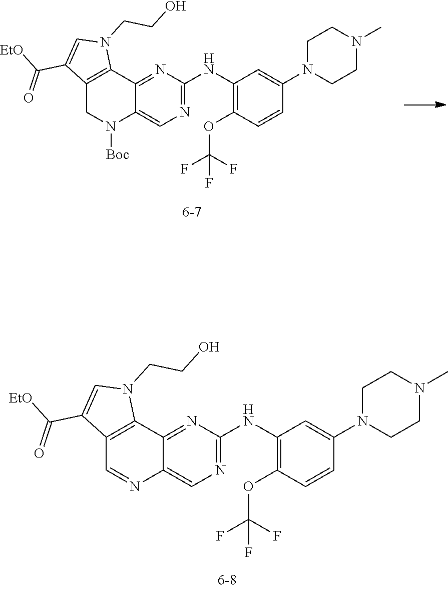 [SMILES: CCOC(=O)c1cn(CCO)c2c1CN(C(=O)OC(C)(C)C)c1cnc(Nc3cc(N4CCN(C)CC4)ccc3OC(F)(F)F)nc1-2 ; CCOC(=O)c1cn(CCO)c2c1cnc1cnc(Nc3cc(N4CCN(C)CC4)ccc3OC(F)(F)F)nc12]

Intermediate 6-7 (0.14 g, 0.21 mmol) was dissolved in dichloromethane (5 mL), trifluoroacetic acid (0.5 mL) was added, and the mixture was reacted at room temperature for 4 h. The reaction liquid was concentrated under reduced pressure to obtain intermediate 6-8 (0.12 g, crude product), which was directly used in the next step.

### 6.8 Synthesis of Compound 6

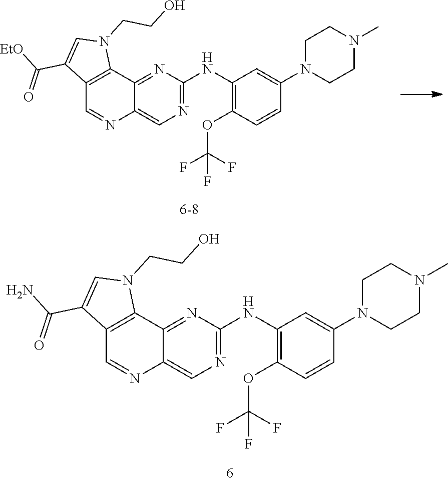 [SMILES: CCOC(=O)c1cn(CCO)c2c1cnc1cnc(Nc3cc(N4CCN(C)CC4)ccc3OC(F)(F)F)nc12 ; CN1CCN(c2ccc(OC(F)(F)F)c(Nc3ncc4ncc5c(C(N)=O)cn(CCO)c5c4n3)c2)CC1]

Intermediate 6-8 (0.12 g, 0.21 mmol) was dissolved in NMP (6 mL), and NH₃/MeOH (5 mL) was added. The mixture was reacted for 48 h under sealed conditions at 80.0° C. The reaction liquid was concentrated under reduced pressure, and the residue was purified by preparative liquid chromatogram (C18 30*150 mm*7 μm; mobile phase: [water (TFA)-acetonitrile]; B %: 10%-80%, 40 min). Compound 6 (0.045 g, yield 40%, yellow solid) was obtained. MS (ESI) m/z=532.2 [M+H]⁺. ¹H NMR (400 MHz, DMSO-d6) δ 9.66 (s, 1H), 9.35 (d, J=1.7 Hz, 2H), 7.97 (s, 1H), 7.68 (s, 1H), 7.27-7.30 (m, 2H), 6.87-6.90 (m, 1H), 4.88-4.90 (m, 2H), 4.65-4.67 (m, 1H), 3.77 (d, J=5.5 Hz, 2H), 3.17-3.20 (m, 4H), 2.45-2.47 (m, 4H), 2.23 (s, 3H).

### Example 7: Synthesis of Compound 7

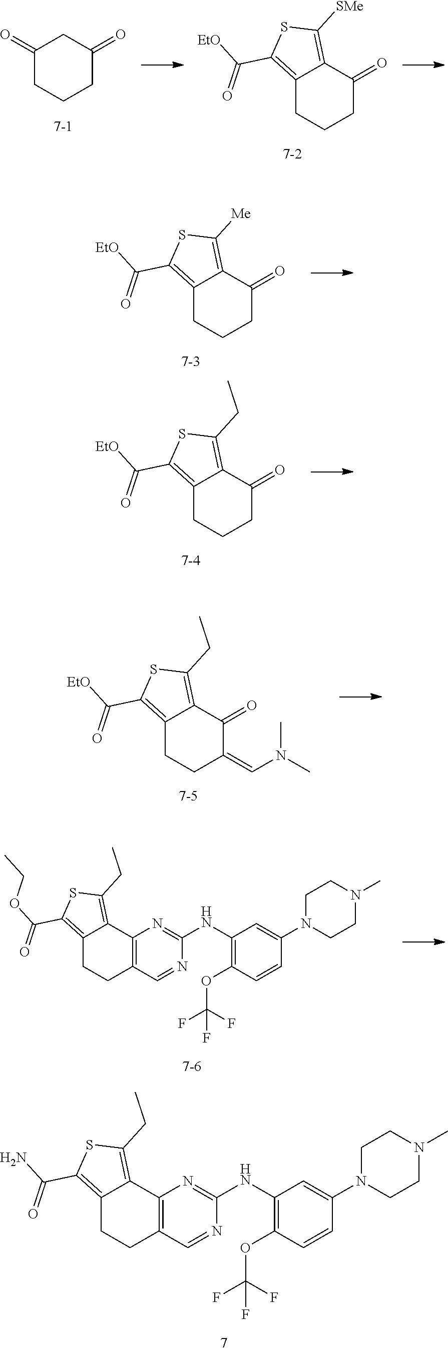

### 7.1 Synthesis of Intermediate 7-2

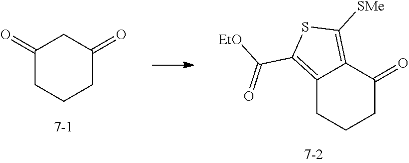 [SMILES: O=C1CCCC(=O)C1 ; CCOC(=O)c1sc(SC)c2c1CCCC2=O]

K₂CO₃ (206 g, 1.50 mol) was dissolved in DMF (450 mL), intermediate 7-1 (55.9 g, 0.50 mol) was added, and the mixture was reacted under stirring at room temperature for 10 min. CS2 (56.9 g, 0.75 mol) was added and the mixture was reacted under stirring at room temperature for 10 min. Then, a solution of ethyl bromoacetate (83.3 g, 0.50 mol) in DMF (500 mL) was added dropwise at 0° C., and the reaction was carried out at 0° C. for 1 h. Finally, a solution of iodomethane (77.8 g, 0.55 mol) in DMF (200 mL) was added dropwise at 0° C., and the reaction was carried out at 0° C. for 0.5 h. The reaction liquid was filtered, the filtrate was poured into water (5 L), and ethyl acetate (1 L×5) was added for extraction. The organic phases were combined, dried over anhydrous sodium sulfate, filtered, and the organic phase was concentrated under reduced pressure. Intermediate 7-2 (720 g, crude product, oil) was obtained and directly used in the next step.

### 7.2 Synthesis of Intermediate 7-3

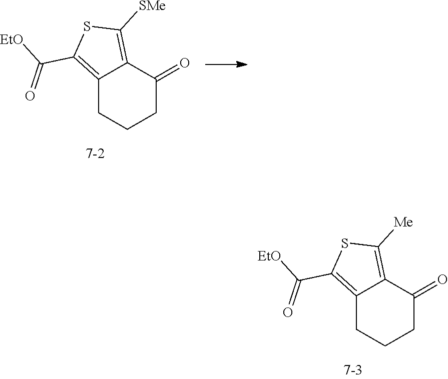 [SMILES: CCOC(=O)c1sc(SC)c2c1CCCC2=O ; CCOC(=O)c1sc(C)c2c1CCCC2=O]

Intermediate 7-2 (53.5 g, 197 mmol) was dissolved in ethanol (650 mL) and water (650 mL), potassium monopersulfate (401 g, 2.39 mol) was added, and the mixture was reacted at 60° C. 16 h. Water (1.1 L) was added, and the mixture was stirred at 50-60° C. for 0.5 h and cooled to 15° C., a solid was precipitated, filtered, and the filter cake was dried. The filter cake was subjected to silica gel column chromatography (ethyl acetate:petroleum ether=0-40%) to obtain the intermediate 7-3 (6.5 g, yield 11%, orange solid). MS (ESI) m/z=303.0 [M+H]⁺.

### 7.3 Synthesis of Intermediate 7-4

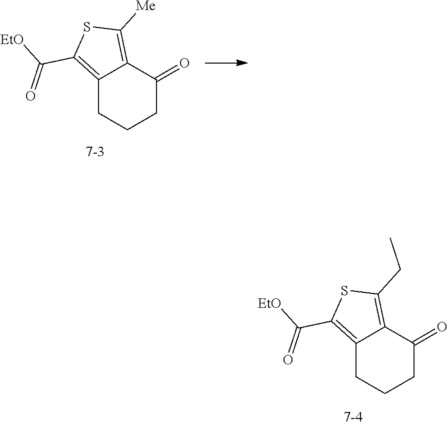 [SMILES: CCOC(=O)c1sc(C)c2c1CCCC2=O ; CCOC(=O)c1sc(CC)c2c1CCCC2=O]

Intermediate 7-3 (9.00 g, 29.8 mmol) was dissolved in tetrahydrofuran (90 mL), and ethylmagnesium bromide (1.00 M, 32.7 mL) was added at 0° C. The mixture was reacted at 35° C. for 16 h. Saturated ammonium chloride (100 mL) was added to quench the reaction, and ethyl acetate (100 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-10%) to obtain intermediate 7-4 (1.47 g, yield 19.5%, white solid). MS (ESI) m/z=253.1 [M+H]⁺.

### 7.4 Synthesis of Intermediate 7-5

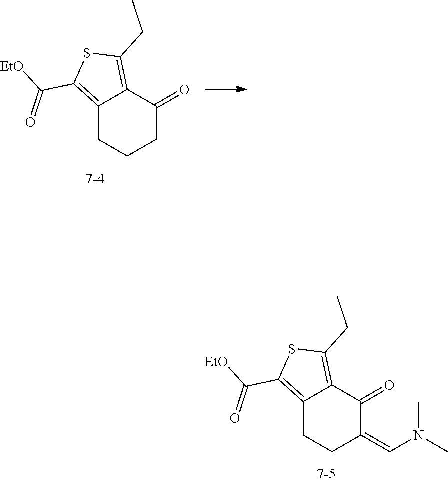 [SMILES: CCOC(=O)c1sc(CC)c2c1CCCC2=O ; CCOC(=O)c1sc(CC)c2c1CC/C(=C/N(C)C)C2=O]

Intermediate 7-4 (0.50 g, 1.98 mmol) was dissolved in DMF-DMA (4.49 g, 37.6 mmol, 5.00 mL) and the mixture was reacted at 100° C. for 16 h. The reaction liquid was concentrated under reduced pressure to obtain intermediate 7-5 (0.75 g, crude product), which was directly used in the next step.

### 7.5 Synthesis of Intermediate 7-6

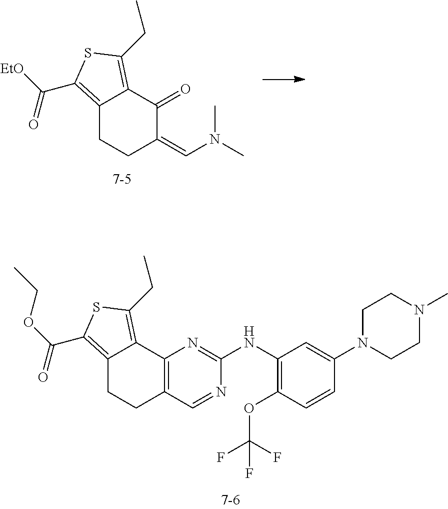 [SMILES: CCOC(=O)c1sc(CC)c2c1CC/C(=C/N(C)C)C2=O ; CCOC(=O)c1sc(CC)c2c1CCc1cnc(Nc3cc(N4CCN(C)CC4)ccc3OC(F)(F)F)nc1-2]

Intermediate 7-5 (0.37 g, 1.20 mmol) and intermediate 1-3 (0.34 g, 1.08 mmol) were dissolved in DMF (5 mL) and the mixture was reacted at 110° C. for 4 h. Water (80 mL) was added to quench the reaction, and ethyl acetate (80 mL×4) was added for extraction. The organic phases were combined, dried over anhydrous sodium sulfate, filtered, and the organic phase was concentrated under reduced pressure. The residue was subjected to silica gel column chromatography (methanol:dichloromethane=0%-7%) to obtain intermediate 7-6 (0.23 g, yield 34.8%, brown solid). MS (ESI) m/z=562.2 [M+H]⁺.

### 7.6 Synthesis of Compound 7

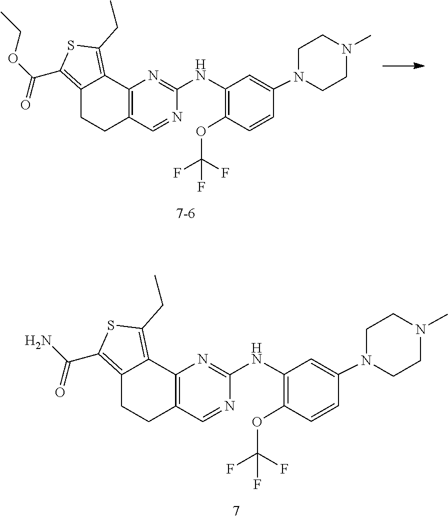 [SMILES: CCOC(=O)c1sc(CC)c2c1CCc1cnc(Nc3cc(N4CCN(C)CC4)ccc3OC(F)(F)F)nc1-2 ; CCc1sc(C(N)=O)c2c1-c1nc(Nc3cc(N4CCN(C)CC4)ccc3OC(F)(F)F)ncc1CC2]

Intermediate 7-6 (100 mg, 178 μmol) was dissolved in NMP (2 mL), and NH₃/MeOH (4.0 mL) was added. The mixture was reacted at 15 Psi and 100.0° C. for 32 h. The reaction liquid was concentrated under reduced pressure, and the residue was purified by preparative liquid chromatogram (C18 75*30 mm*3 μm; mobile phase: [H₂O(NH₃H₂O+NH₄HCO₃)-ACN]; B %: 40.0%-58.0%, 14.0 min). Compound 7 (11.6 mg, yield 95.0%, white solid) was obtained. MS (ESI) m/z=533.2 [M+H]⁺. ¹H NMR (400 MHz, DMSO-d6) δ 8.64 (s, 1H), 8.32 (s, 1H), 7.39 (br, 2H), 7.26-7.13 (m, 2H), 6.83-6.74 (m, 1H), 3.20-3.03 (m, 8H), 2.70 (br, 2H), 2.48-2.38 (m, 5H), 2.21 (s, 3H), 1.04 (t, J=7.3 Hz, 3H).

### Example 8: Synthesis of Compound 8

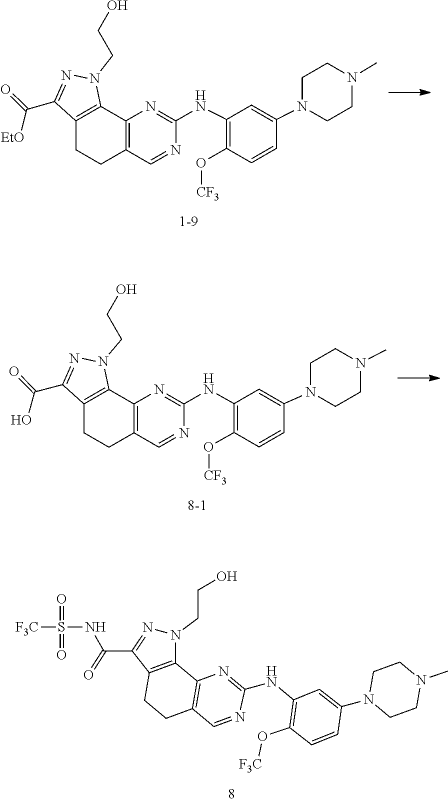 [SMILES: CCOC(=O)c1nn(CCO)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)O)nn3CCO)CC4)c2)CC1 ; CN1CCN(c2ccc(OC(F)(F)F)c(Nc3ncc4c(n3)-c3c(c(C(=O)NS(=O)(=O)C(F)(F)F)nn3CCO)CC4)c2)CC1]

### 8.1 Synthesis of Intermediate 8-1

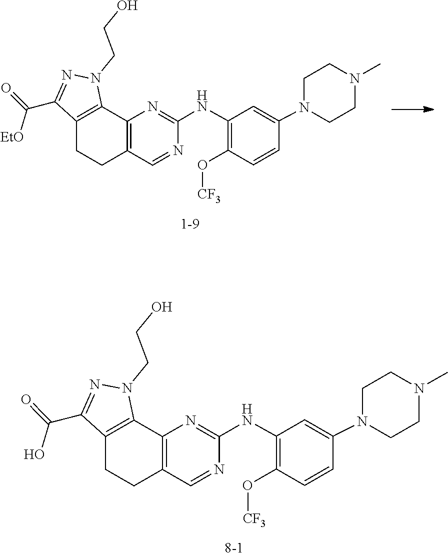 [SMILES: CCOC(=O)c1nn(CCO)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)O)nn3CCO)CC4)c2)CC1]

Compound 1-9 (550 mg, 0.766 mmol) was dissolved in ethanol (5 mL), and LiOH·H₂O (2 M, 1.53 mL) was added dropwise. The mixture was reacted at room temperature for 5 h. Water (10 mL) was added to quench the reaction, ethyl acetate (20 mL) was added for extraction, the aqueous phase was collected and adjusted to pH 3-4 to precipitate a solid. The resulting product was filtered, the filter cake was dried to obtain intermediate 8-1 (290 mg, crude product), which was directly used in the next step. MS (ESI) m/z=534.3 [M+H]⁺.

### 8.2 Synthesis of Compound 8

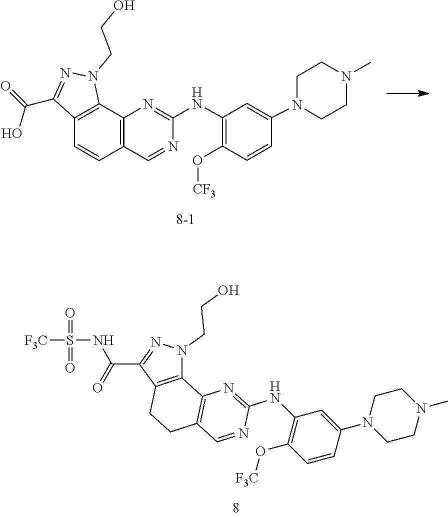 [SMILES: CN1CCN(c2ccc(OC(F)(F)F)c(Nc3ncc4ccc5c(C(=O)O)nn(CCO)c5c4n3)c2)CC1 ; CN1CCN(c2ccc(OC(F)(F)F)c(Nc3ncc4c(n3)-c3c(c(C(=O)NS(=O)(=O)C(F)(F)F)nn3CCO)CC4)c2)CC1]

Intermediate 8-1 (53.1 mg, 0.10 mmol) was dissolved in dichloromethane (5 mL), trifluoromethanesulfonamide (89.4 mg, 0.60 mmol), EDCI (76.8 mg, 0.40 mmol) and DMAP (48.8 mg, 0.40 mmol) were added. The mixture was stirred at room temperature for 16 h. The mixture was diluted with dichloromethane (10 mL), water (20.0 mL*3) was added for extraction, the organic phase was dried over anhydrous sodium sulfate, and filtered, and the organic phase was concentrated under reduced pressure. The residue was subjected to column chromatography (C18 150*30 mm*5 μm; mobile phase: [H₂O(NH₄HCO₃)-ACN]; B %: 10%-60%, 12 min), compound 8 (21.7 mg, yield 32.6%, white solid) was obtained. MS (ESI) m/z=665.3 [M+H]⁺. ¹H NMR (400 MHz DMSO-d₆) δ 8.82 (s, 1H), 8.29 (s, 1H), 7.23-7.21 (m, 2H), 6.80-6.78 (m, 1H), 4.60 (m, 3H), 3.57 (s, 2H), 3.19 (s, 4H), 2.98-2.94 (m, 2H), 2.78-2.74 (m, 2H), 2.67-2.65 (m, 4H), 2.36-2.33 (m, 3H).

### Example 9: Synthesis of Compound 9

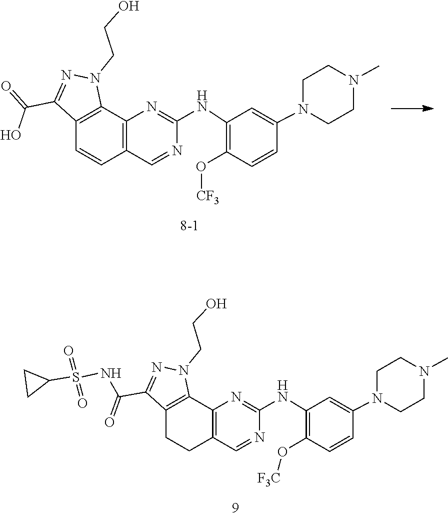 [SMILES: CN1CCN(c2ccc(OC(F)(F)F)c(Nc3ncc4ccc5c(C(=O)O)nn(CCO)c5c4n3)c2)CC1 ; CN1CCN(c2ccc(OC(F)(F)F)c(Nc3ncc4c(n3)-c3c(c(C(=O)NS(=O)(=O)C5CC5)nn3CCO)CC4)c2)CC1]

A method similar to Example 8 was used to prepare compound 9 (13 mg, yield 38.5%, white solid) from intermediate 8-1 and cyclopropanesulfonamide. MS (ESI) m/z=637.2 [M+H]⁺. ¹H NMR (400 MHz DMSO-d₆) δ 10.99 (br, 1H), 8.99 (s, 1H), 8.36 (s, 1H), 7.28-7.25 (m, 2H), 6.85-6.82 (m, 1H), 4.66 (m, 3H), 3.68-3.66 (m, 2H), 3.33-3.29 (m, 4H), 3.07-3.03 (m, 1H), 3.00-2.92 (m, 6H), 2.84-2.80 (m, 2H), 2.55-2.50 (m, 3H), 1.10-1.00 (m, 4H).

### Example 10: Synthesis of Compound 10

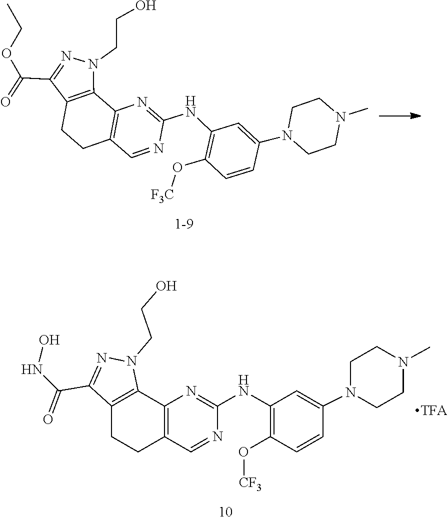 [SMILES: CCOC(=O)c1nn(CCO)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)NO)nn3CCO)CC4)c2)CC1]

Na (40.9 mg, 1.78 mmol) was dissolved in methanol (1.8 mL), HONH₂·HCl (37.1 mg, 0.534 mmol) was added, and the mixture was stirred at room temperature for 1 h under nitrogen protection. Intermediate 1-9 (100 mg, 0.178 mmol) was added and the mixture was reacted at 65° C. for 0.5 h. The reaction liquid was filtered, and the filtrate was concentrated under reduced pressure. The residue was subjected to column chromatography (C18 150*40 mm*10 μm; mobile phase: [H₂O(TFA)-ACN]; B %: 10.0%-50.0%, 10 min), the compound 10 (12.0 mg, 11.3% yield, trifluoroacetate, white solid) was obtained. MS (ESI) m/z=549.1 [M+H]⁺. ¹H NMR (400 MHz DMSO-d₆) δ 10.89 (s, 1H), 9.86-9.65 (m, 1H), 9.00 (s, 1H), 8.34 (s, 1H), 7.35-7.26 (m, 2H), 6.85-6.90 (m, 1H), 4.62 (br, 2H), 3.84 (br, 2H), 3.64 (br, 3H), 3.20-3.10 (m, 2H), 2.94 (br, 4H), 2.86 (s, 3H), 2.83-2.77 (m, 2H), 2.55 (t, J=5.5 Hz, 2H).

### Example 11: Synthesis of Compound 11

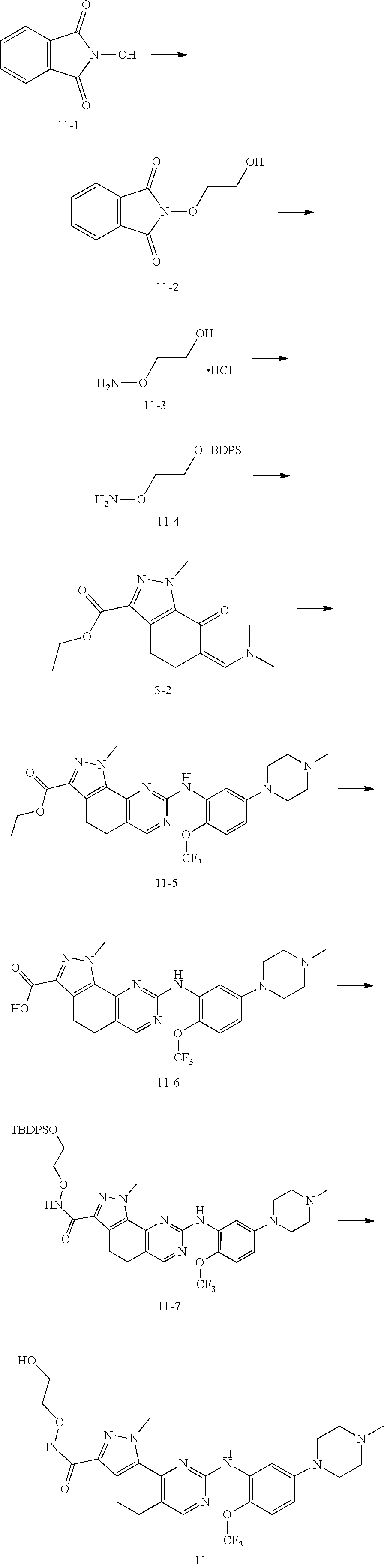

### 11.1 Synthesis of Intermediate 11-2

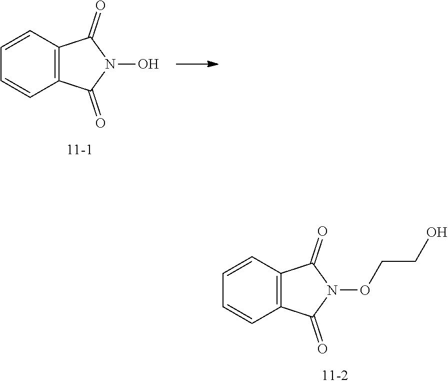 [SMILES: O=C1c2ccccc2C(=O)N1O ; O=C1c2ccccc2C(=O)N1OCCO]

Intermediate 11-1 (10.0 g, 61.3 mmol) was dissolved in acetonitrile (20 mL), triethylamine (7.43 g, 73.4 mmol) and 2-bromoethanol (9.18 g, 73.4 mmol) were added under ice bath conditions. The mixture was reacted at 80° C. for 8 h. Water (200 mL) was added to quench the reaction, and ethyl acetate (200 mL×3) was added for extraction. The organic phases were combined, dried over anhydrous sodium sulfate, filtered, and the organic phase was concentrated under reduced pressure. The intermediate 11-2 (4.5 g, crude product) was obtained and used directly in the next step. MS (ESI) m/z=207.9 [M+H]⁺.

### 11.2 Synthesis of Intermediate 11-3

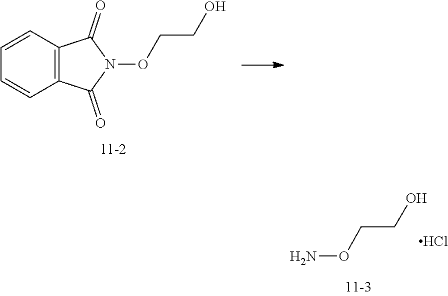 [SMILES: O=C1c2ccccc2C(=O)N1OCCO ; NOCCO ; Cl]

Intermediate 11-2 (2.00 g, 9.65 mmol) was dissolved in dichloromethane (40 mL), N₂H₄·H₂O (1.02 g, 19.3 mmol, 987 μL) was added. The mixture was reacted at room temperature for 12 h. HCl/dioxane (2 mL, 4 M) was added and the mixture was stirred at room temperature to precipitate a solid. The resulting product was filtered, the filter cake was dried to obtain intermediate 11-3 (0.69 g, crude product), which was directly used in the next step.

### 11.3 Synthesis of Intermediate 11-4

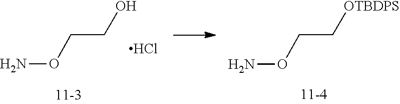

Intermediate 11-3 (2.32 g, 8.43 mmol) was dissolved in dichloromethane (40 mL), DMAP (79.2 mg, 0.648 mmol), imidazole (1.10 g, 16.2 mmol) and tert-butyl diphenylchlorosilane (1.78 g, 6.48 mmol) were added. The mixture was reacted at room temperature for 16 h. The reaction liquid was concentrated under reduced pressure, and the residue was subjected to silica gel column chromatography (ethyl acetate:petroleum ether=0-20%) to obtain intermediate 11-4 (0.23 g, yield 11.3%, oil). MS (ESI) m/z=316.0 [M+H]⁺.

### 11.4 Synthesis of Intermediate 11-5

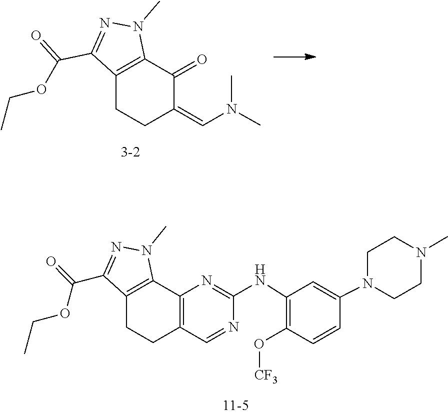 [SMILES: CCOC(=O)c1nn(C)c2c1CC/C(=C/N(C)C)C2=O ; CCOC(=O)c1nn(C)c2c1CCc1cnc(Nc3cc(N4CCN(C)CC4)ccc3OC(F)(F)F)nc1-2]

Intermediate 3-2 (800 mg, 2.88 mmol) was dissolved in DMF (20 mL), intermediate 1-3 (915 mg, 2.88 mmol) was added, and the mixture was reacted at 110° C. for 16 h. Water (50 mL) was added to quench the reaction, ethyl acetate (50 mL×3) was added for extraction, the organic phases were combined, dried over anhydrous sodium sulfate, and filtered, the organic phase was concentrated under reduced pressure, and the residue was subjected to silica gel column chromatograph (methanol:dichloromethane=0-10%). The intermediate 11-5 (738 mg, yield 48.1%, light yellow solid) was obtained. MS (ESI) m/z=532.2 [M+H]⁺.

### 11.5 Synthesis of Intermediate 11-6

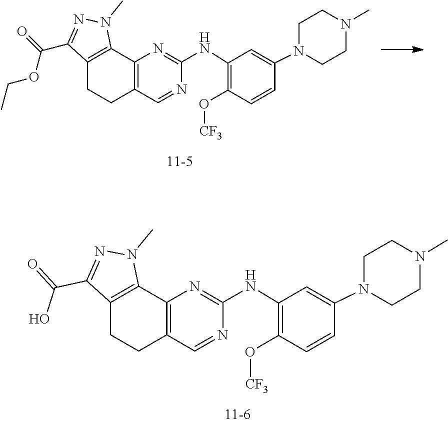 [SMILES: CCOC(=O)c1nn(C)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)O)nn3C)CC4)c2)CC1]

Compound 11-5 (660 mg, 1.24 mmol) was dissolved in methanol (5 mL) and tetrahydrofuran (5 mL), and a solution of LiOH·H₂O (140 mg, 3.35 mmol) in water (5 mL) was added. The mixture was reacted at room temperature for 16 h. Water (10 mL) was added to quench the reaction, ethyl acetate (20 mL) was added for extraction, the aqueous phase was collected, and adjusted to pH 3-4 with dilute hydrochloric acid to precipitate a solid. The resulting product was filtered, the filter cake was dried to obtain intermediate 11-6 (531 mg, crude product), which was directly used in the next step. MS (ESI) m/z=504.1 [M+H]⁺.

### 11.6 Synthesis of Intermediate 11-7

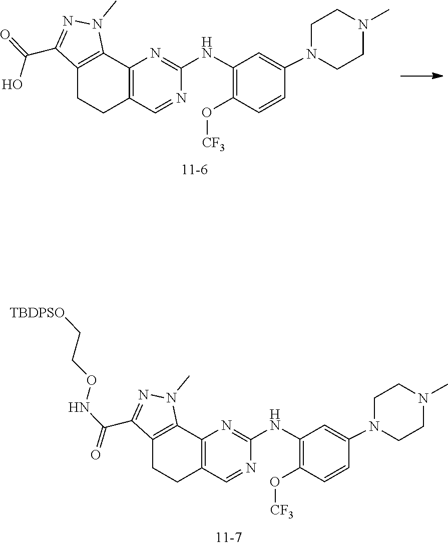

Intermediate 11-6 (125 mg, 397 μmol) was dissolved in DMF (10 mL), CDI (32.2 mg, 199 μmol) was added, and the mixture was stirred at 50° C. for 2 h. Intermediate 11-4 (50 mg, 99.3 μmol) was added and the mixture was reacted at 50° C. for 46 h. The reaction liquid was poured into water (100 mL), and a solid was precipitated. The obtained product was filtered, the filter cake was collected, and silica gel column chromatography (ethyl acetate:petroleum ether=30%) was performed. The intermediate 11-7 (2.77 g, yield 55.5%, yellow solid) was obtained. MS (ESI) m/z=801.3 [M+H]⁺.

### 11.7 Synthesis of Compound 11

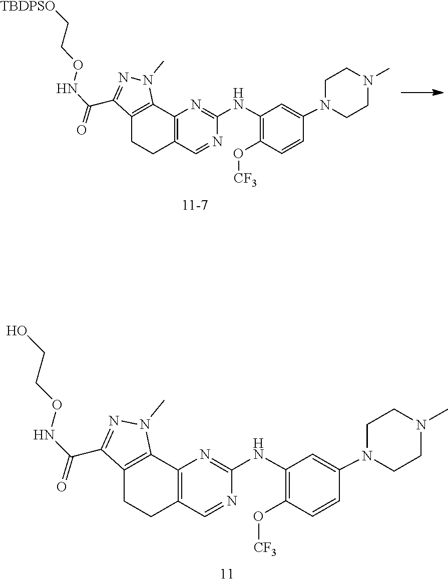

Intermediate 11-7 (100 mg, 125 μmol) was dissolved in dichloromethane (10 mL), Et₃N·3HF (20.1 mg, 125 μmol) was added. The mixture was reacted at room temperature for 12 h. The reaction liquid was concentrated under reduced pressure, and the residue was subjected to column chromatography (C18 30*150 mm*7 μm; mobile phase: [H₂O(NH₄HCO₃)-MeOH]; B %: 30%-80%, 40 min) to obtain compound 11 (33.0 mg, yield 46.0%, white solid). MS (ESI) m/z=563.2 [M+H]⁺. ¹H NMR (400 MHz, DMSO-d6) δ 8.90-8.98 (m, 1H), 8.34-8.37 (m, 1H), 7.25-7.32 (m, 1H), 7.14-7.23 (m, 1H), 6.76 (dd, J=9.2, 2.8 Hz, 1H), 4.16 (s, 3H), 3.86 (s, 2H), 3.57 (s, 3H), 3.08-3.22 (m, 4H), 2.90-2.99 (m, 2H), 2.76-2.86 (m, 3H), 2.42-2.47 (m, 4H), 2.21 (s, 3H).

### Example 12: Synthesis of Compound 12

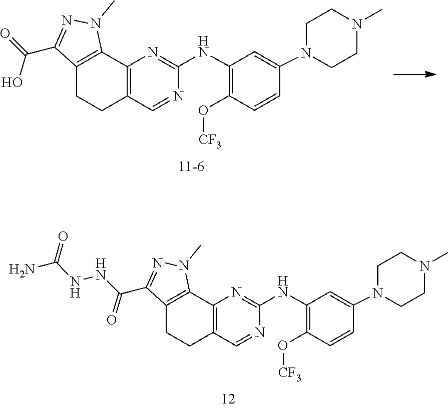 [SMILES: CN1CCN(c2ccc(OC(F)(F)F)c(Nc3ncc4c(n3)-c3c(c(C(=O)O)nn3C)CC4)c2)CC1 ; CN1CCN(c2ccc(OC(F)(F)F)c(Nc3ncc4c(n3)-c3c(c(C(=O)NNC(N)=O)nn3C)CC4)c2)CC1]

A method similar to Example 11 was used to prepare compound 12 from the reaction of intermediate 11-6 and semicarbazide. MS (ESI) m/z=561.2 [M+H]⁺. ¹H NMR (400 MHz, CD₃OD) δ 8.30 (s, 1H), 7.67 (s, 1H), 7.24 (d, J=8.8 Hz, 1H), 6.78-6.81 (m, 1H), 4.28 (s, 3H), 3.37-3.35 (m, 4H), 3.13-3.05 (m, 6H), 2.88 (t, J=7.6 Hz, 2H), 2.68 (s, 3H).

### Example 13: Synthesis of Compound 13

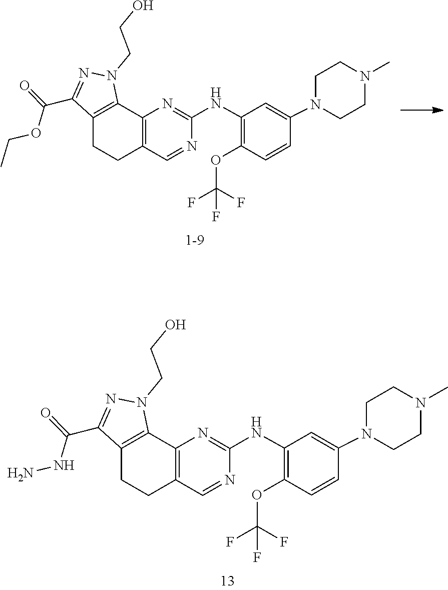 [SMILES: CCOC(=O)c1nn(CCO)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)NN)nn3CCO)CC4)c2)CC1]

Intermediate 1-9 (20 mg, 0.036 mmol) was dissolved in methanol (1 mL), and N₂H₄—H₂O (54.6 mg, 1.07 mmol, 53 μL) was added. The mixture was reacted at 70.0° C. for 36 h. The reaction liquid was concentrated under reduced pressure, and the residue was purified by preparative liquid chromatogram (C18 75*30 mm*3 μm; mobile phase: [H₂O(NH₃H₂O+NH₄HCO₃)-ACN]; B %: 21%-41%, 10 min). Compound 13 (6.0 mg, yield 30.5%, white solid) was obtained. MS (ESI) m/z=548.3 [M+H]⁺. ¹H NMR (400 MHz, CD₃OD) δ 8.28-8.32 (m, 1H), 7.46-7.54 (m, 1H), 7.20-7.24 (m, 1H), 6.80-6.84 (m, 1H), 4.77 (t, J=5.6 Hz, 3H), 4.56-4.62 (m, 2H), 4.51-4.63 (m, 3H), 3.75-3.85 (m, 2H), 3.21-3.27 (m, 4H), 2.98-3.11 (m, 2H), 2.85-2.92 (m, 2H), 2.59-2.66 (m, 4H), 2.34-2.39 (m, 3H).

### Example 14: Synthesis of Compound 17

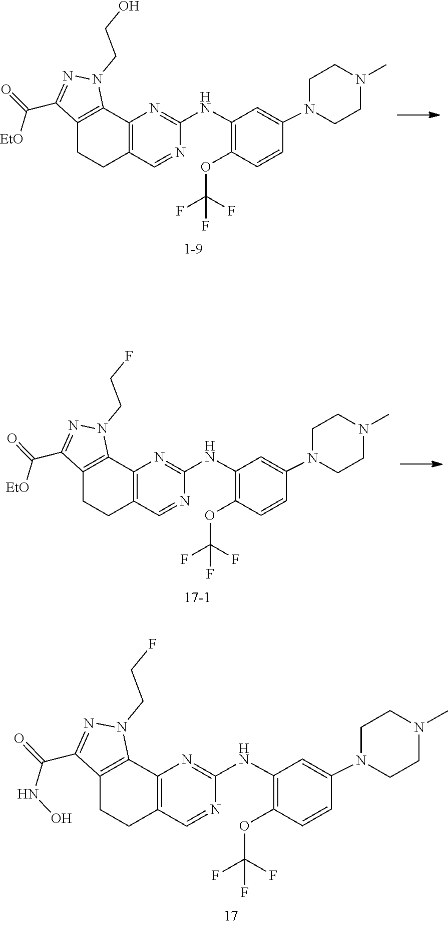 [SMILES: CCOC(=O)c1nn(CCO)c2c1CCc1cnc(Nc3cc(N4CCN(C)CC4)ccc3OC(F)(F)F)nc1-2 ; CCOC(=O)c1nn(CCF)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)NO)nn3CCF)CC4)c2)CC1]

### 14.1 Synthesis of Intermediate 17-1

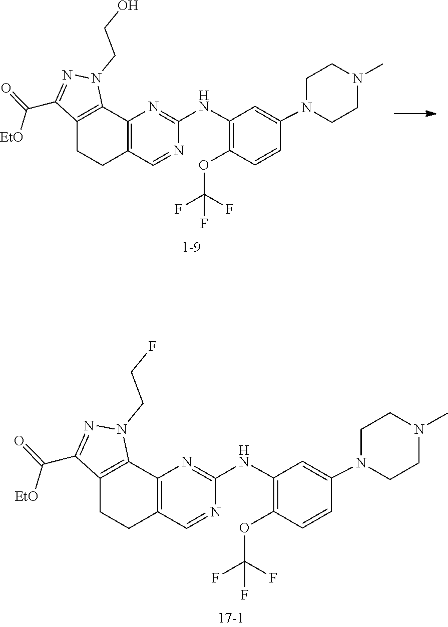 [SMILES: CCOC(=O)c1nn(CCO)c2c1CCc1cnc(Nc3cc(N4CCN(C)CC4)ccc3OC(F)(F)F)nc1-2 ; CCOC(=O)c1nn(CCF)c2c1CCc1cnc(Nc3cc(N4CCN(C)CC4)ccc3OC(F)(F)F)nc1-2]

Intermediate 1-9 (200 mg, 0.36 mmol) was dissolved in dichloromethane (20 mL) and DAST (293 mg, 1.82 mmol) was added dropwise at −78° C. The mixture was reacted at room temperature for 12 h. The reaction liquid was cooled to room temperature, water (70 mL) was added to quench the reaction, and dichloromethane (50 mL×3) was added for extraction. The organic phases were combined, dried over anhydrous sodium sulfate, filtered, and the organic phase was concentrated under reduced pressure. The residue was purified by preparative liquid chromatogram (C18 30*150 mm*5 μm; mobile phase: [water (NH₄HCO₃)-acetonitrile]; B %: 45%-82%, 12 min) to obtain intermediate 17-1 (170 mg, yield 84.8%, brown solid). MS (ESI) m/z=564.2 [M+H]⁺.

### 14.2 Synthesis of Compound 17

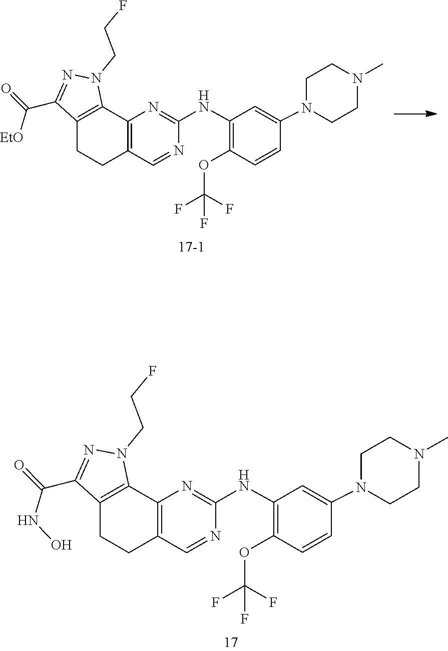 [SMILES: CCOC(=O)c1nn(CCF)c2c1CCc1cnc(Nc3cc(N4CCN(C)CC4)ccc3OC(F)(F)F)nc1-2 ; CN1CCN(c2ccc(OC(F)(F)F)c(Nc3ncc4c(n3)-c3c(c(C(=O)NO)nn3CCF)CC4)c2)CC1]

Na (40.9 mg, 1.78 mmol) was dissolved in methanol (1.8 mL), HONH₂·HCl (37.1 mg, 0.534 mmol) was added, and the mixture was stirred at room temperature for 1 h under nitrogen protection. Intermediate 17-1 (100 mg, 0.178 mmol) was added and the mixture was reacted at 65° C. for 0.5 h. The reaction liquid was filtered, and the filtrate was concentrated under reduced pressure. The residue was subjected to column chromatography (C18 150*40 mm*10 μm; mobile phase: [H₂O(TFA)-ACN]; B %: 10.0%-50.0%, 10 min), the compound 17 (11.1 mg, 11.3% yield, trifluoroacetate, white solid) was obtained. MS (ESI) m/z=551.2 [M+H]⁺. ¹H NMR (400 MHz DMSO-d₆) δ 10.90 (s, 1H), 9.52-9.82 (m, 1H), 9.10 (s, 1H), 8.36 (s, 1H), 7.23-7.36 (m, 2H), 6.88 (dd, J=9.01, 2.75 Hz, 1H), 4.78-4.94 (m, 2H), 4.65 (t, J=4.63 Hz, 1H), 4.53 (t, J=4.44 Hz, 1H), 3.84 (d, J=13.01 Hz, 2H), 3.07-3.26 (m, 3H), 2.93-3.01 (m, 4H), 2.78-2.92 (m, 6H).

### Example 15: Synthesis of Compound 18

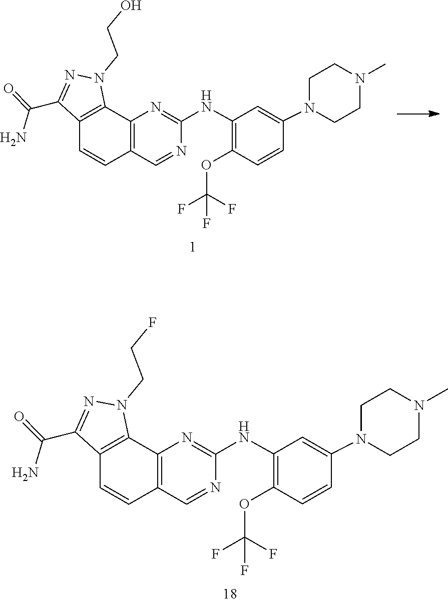 [SMILES: CN1CCN(c2ccc(OC(F)(F)F)c(Nc3ncc4ccc5c(C(N)=O)nn(CCO)c5c4n3)c2)CC1 ; CN1CCN(c2ccc(OC(F)(F)F)c(Nc3ncc4ccc5c(C(N)=O)nn(CCF)c5c4n3)c2)CC1]

Compound 1 (50 mg, 94.2 μmol) was dissolved in dichloromethane (5 mL), and DAST (77.5 mg, 481 μmol) was added dropwise at −78° C. The mixture was reacted at room temperature for 16 h. The reaction liquid was cooled to room temperature, water (20 mL) was added to quench the reaction, and dichloromethane (10 mL×3) was added for extraction. The organic phases were combined, dried over anhydrous sodium sulfate, filtered, and the organic phase was concentrated under reduced pressure. The residue was purified by preparative liquid chromatogram (C18 30*150 mm*5 μm; mobile phase: [water (NH₄HCO₃)-acetonitrile]; B %: 27%-73%, 12 min) to obtain compound 18 (5 mg, yield 10%, brown solid). MS (ESI) m/z=533.2 [M+H]⁺. ¹H NMR: (400 MHz MeOD) δ 9.21 (s, 1H), 8.07 (d, J=8.0 Hz, 1H), 7.57-7.49 (m, 2H), 7.32-7.30 (d, J=8.0 Hz, 1H), 6.93-6.91 (m, 1H), 5.30-5.21 (m, 2H), 4.80-4.66 (m, 2H), 3.37 (m, 4H), 2.96 (m, 4H), 2.60 (s, 3H).

### Example 16: Synthesis of Compound 19

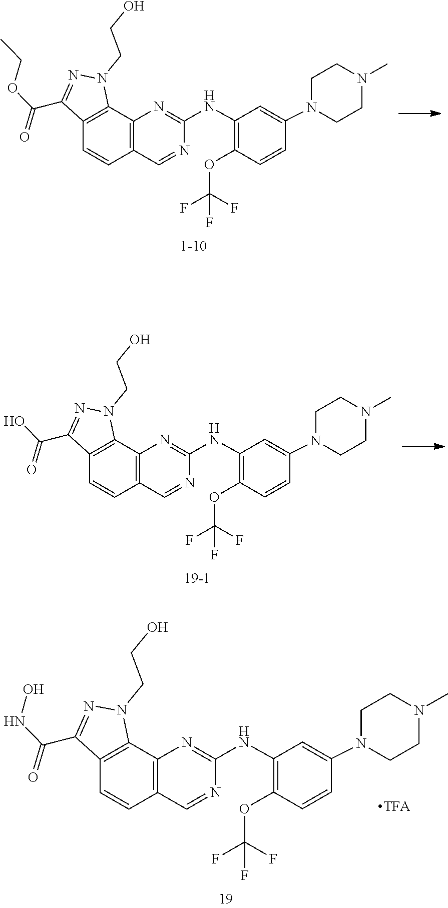 [SMILES: CCOC(=O)c1nn(CCO)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)nn(CCO)c5c4n3)c2)CC1 ; CN1CCN(c2ccc(OC(F)(F)F)c(Nc3ncc4ccc5c(C(=O)NO)nn(CCO)c5c4n3)c2)CC1]

### 16.1 Synthesis of Intermediate 19-1

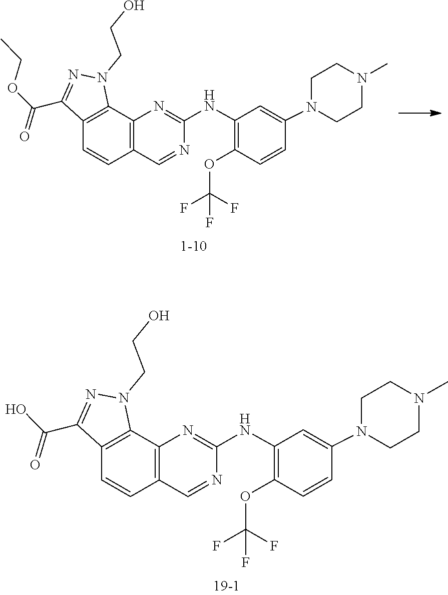 [SMILES: CCOC(=O)c1nn(CCO)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)nn(CCO)c5c4n3)c2)CC1]

Intermediate 1-10 (100 mg, 0.178 mmol) was dissolved in methanol (2 mL) and lithium hydroxide aqueous solution (1 M, 890 μL) was added. The mixture was reacted at room temperature for 6 h. The pH was adjusted to 5-7 with hydrochloric acid at 0° C., the residue was purified by preparative liquid chromatogram (C18 150*30 mm*5 μm; mobile phase: [water (TFA)-acetonitrile]; B %: 10%-50%, 10 min) to obtain intermediate 19-1 (37 mg, yield 39%, brown solid). MS (ESI) m/z=532.3 [M+H]⁺.

### 16.2 Synthesis of Compound 19

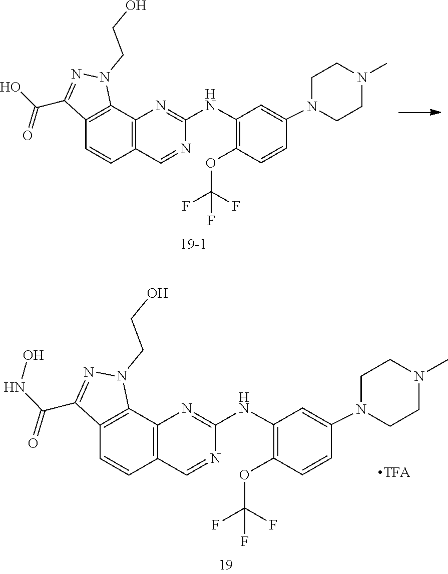 [SMILES: CN1CCN(c2ccc(OC(F)(F)F)c(Nc3ncc4ccc5c(C(=O)O)nn(CCO)c5c4n3)c2)CC1 ; CN1CCN(c2ccc(OC(F)(F)F)c(Nc3ncc4ccc5c(C(=O)NO)nn(CCO)c5c4n3)c2)CC1]

Intermediate 19-1 (37.0 mg, 69.6 μmol), HATU (29.1 mg, 76.6 μmol) and DIEA (36.0 mg, 278 μmol) were dissolved in DMF (2 mL), and hydroxylamine hydrochloride (9.68 mg, 139 μmol) was added. The mixture was reacted at room temperature for 2 h. The reaction liquid was concentrated under reduced pressure, and the residue was purified by preparative liquid chromatogram (C18 150*30 mm*5 μm; mobile phase: [water (TFA)-acetonitrile]; B %: 5%-45%, 10 min) to obtain the compound 19 (17 mg, 37% yield, brown solid, trifluoroacetate salt). MS (ESI) m/z=547.2 [M+H]⁺. ¹H NMR (DMSO-d₆) δ 10.89 (s, 1H), 9.86-9.65 (m, 1H), 9.00 (s, 1H), 8.34 (s, 1H), 7.35-7.26 (m, 2H), 6.86 (dd, J=2.9, 9.0 Hz, 1H), 4.62 (br t, J=5.3 Hz, 2H), 3.84 (br d, J=12.5 Hz, 2H), 3.64 (br t, J=5.4 Hz, 3H), 3.20-3.10 (m, 2H), 2.94 (br t, J=7.8 Hz, 4H), 2.86 (s, 3H), 2.83-2.77 (m, 2H), 2.55 (t, J=5.5 Hz, 2H).

### Example 17: Synthesis of Compound 20

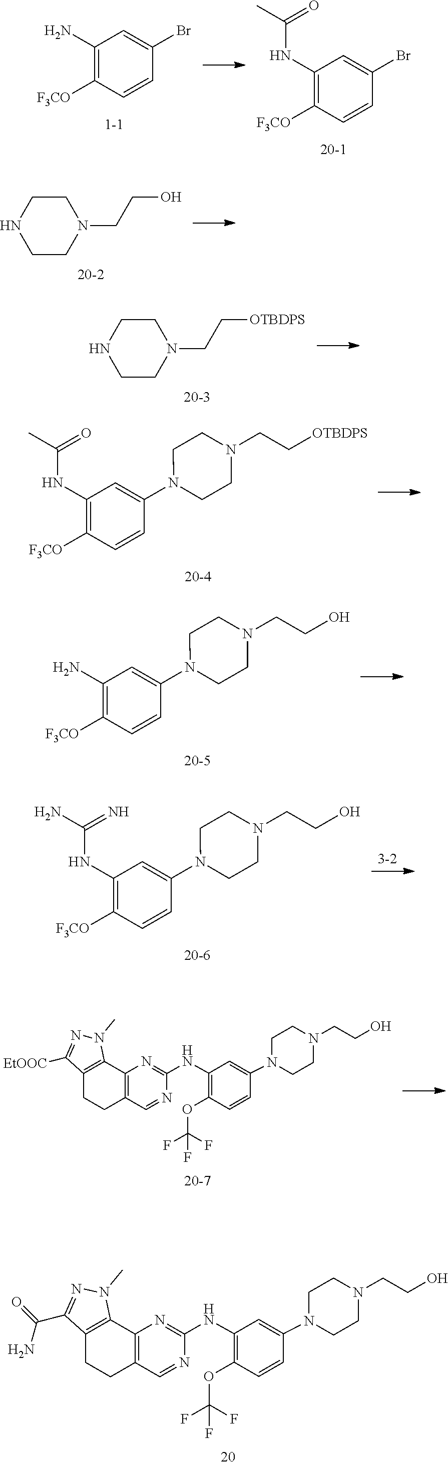

### 17.1 Synthesis of Intermediate 20-1

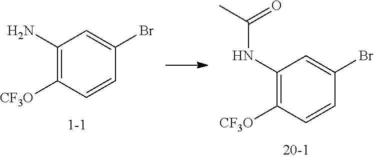

Intermediate 1-1 (10 g, 39.1 mmol) was dissolved in acetic anhydride (9.97 g, 97.6 mmol) and the mixture was reacted at room temperature for 12 h. The reaction liquid was concentrated under reduced pressure. The residue was subjected to silica gel column chromatography (methanol/dichloromethane=0%-10%) to obtain intermediate 20-1 (10.5 g, yield 89.3%, white solid). MS (ESI) m/z=297.9 [M+H]⁺.

### 17.2 Synthesis of Intermediate 20-3

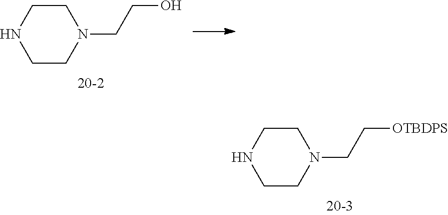

Intermediate 20-2 (2.50 g, 19.2 mmol) was dissolved in dichloromethane (50 mL), and TBDPSCl (6.33 g, 23.0 mmol), DMAP (0.23 g, 1.92 mmol) and imidazole (1.96 g, 28.8 mmol) were added. The mixture was reacted at room temperature for 12 h. The reaction liquid was concentrated under reduced pressure, and the residue was subjected to silica gel column chromatography (methanol/dichloromethane=0%-10%) to obtain intermediate 20-3 (6.5 g, yield 91%, yellow solid). MS (ESI) m/z=369.1 [M+H]⁺.

### 17.3 Synthesis of Intermediate 20-4

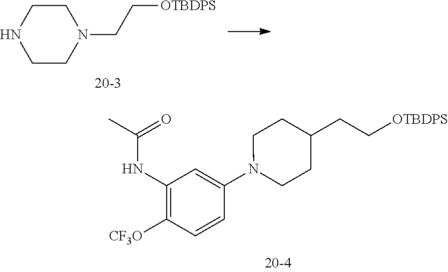

Intermediate 20-3 (3.56 g, 9.66 mmol) was dissolved in tetrahydrofuran (50 mL), LiHMDS (1 M, 18 mL) was added at 0° C., and the mixture was stirred at 0° C. for 1 h. DavePhos (0.317 g, 0.805 mmol), Pd₂(dba)₃ (368.68 mg, 0.403 mmol) and intermediate 20-1 (2.4 g, 8.05 mmol) were added. The mixture was reacted at 80° C. for 12 h. The reaction liquid was cooled to room temperature, water (100 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-10%) to obtain intermediate 20-4 (2.2 g, yield 46.3%, yellow oil). MS (ESI) m/z=586.8 [M+H]⁺.

### 17.4 Synthesis of Intermediate 20-5

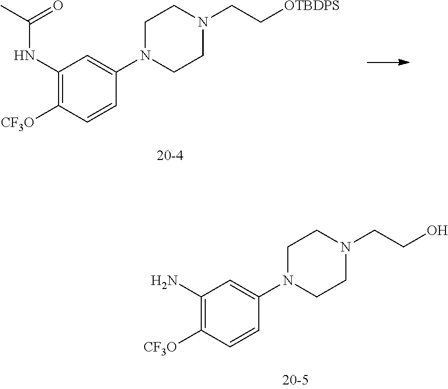

Intermediate 20-4 (2.4 g, 4.10 mmol) was dissolved in ethanol (20 mL) and hydrochloric acid (12 M, 112 mL) was added. The mixture was reacted at 80° C. for 12 h. The reaction liquid was cooled to room temperature and concentrated under reduced pressure. The intermediate 20-5 (1.8 g, crude product) was obtained and used directly in the next step. MS (ESI) m/z=306.1 [M+H]⁺.

### 17.5 Synthesis of Intermediate 20-6

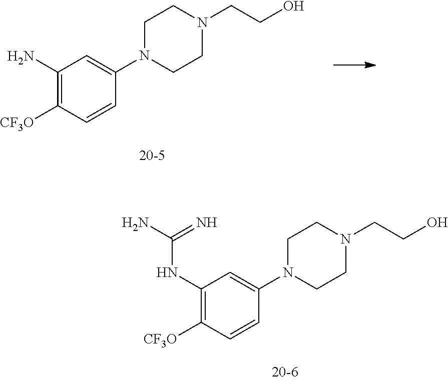

Intermediate 20-5 (1.8 g, 5.90 mmol) was dissolved in hydrochloric acid (12 M, 107 mL) and water (107 mL), and cyanamide (3.72 g, 88.44 mmol) was added. The mixture was reacted at 65° C. for 48 h. The reaction liquid was cooled to room temperature, sodium hydroxide solution (1 M) was added to adjust pH=13, and ethyl acetate (100 mL×3) was added for extraction. The organic phases were combined, dried over anhydrous sodium sulfate, filtered, and the organic phase was concentrated under reduced pressure.

The intermediate 20-6 (1.2 g, crude product) was obtained and used directly in the next step. MS (ESI) m/z=348.1 [M+H]⁺.

### 17.6 Synthesis of Intermediate 20-7

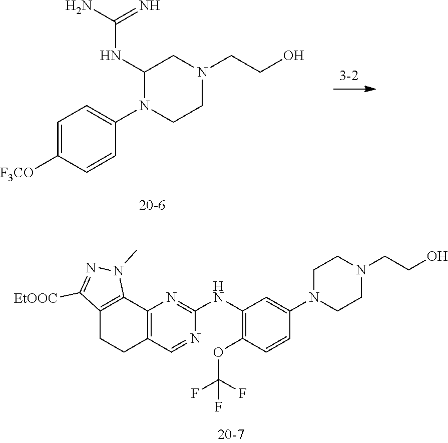 [SMILES: N=C(N)NC1CN(CCO)CCN1c1ccc(OC(F)(F)F)cc1 ; CCOC(=O)c1nn(C)c2c1CCc1cnc(Nc3cc(N4CCN(CCO)CC4)ccc3OC(F)(F)F)nc1-2]

Intermediate 20-6 (1.2 g, 3.45 mmol) and intermediate 3-2 (0.80 g, 2.88 mmol) were dissolved in DMF (10 mL), and the mixture was reacted at 110° C. for 16 h. The reaction liquid was quenched with water (50 mL), and extracted with ethyl acetate (50 mL×3). The organic phases were combined, dried over anhydrous sodium sulfate, filtered, and the organic phase was concentrated under reduced pressure. The residue was subjected to silica gel column chromatography (methanol/dichloromethane=0%-10%) to obtain intermediate 20-7 (0.21 g, yield 13.2%, yellow oil). MS (ESI) m/z=562.2 [M+H]⁺.

### 17.7 Synthesis of Compound 20

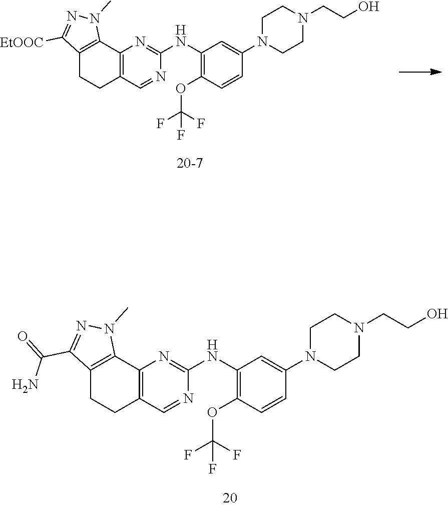 [SMILES: CCOC(=O)c1nn(C)c2c1CCc1cnc(Nc3cc(N4CCN(CCO)CC4)ccc3OC(F)(F)F)nc1-2 ; Cn1nc(C(N)=O)c2c1-c1nc(Nc3cc(N4CCN(CCO)CC4)ccc3OC(F)(F)F)ncc1CC2]

Intermediate 20-7 (150 mg, 267 μmol) was dissolved in a solution of ammonia in methanol (75 mL). The mixture was reacted for 48 h under sealed conditions at 85° C. The reaction liquid was concentrated under reduced pressure, and the residue was subjected to column chromatography (C18 75*30 mm*3 μm; mobile phase: [H₂O(NH₄HCO₃)-ACN]; B %: 27%-62%, 12 min). Compound 20 (21 mg, yield 15%, yellow solid) was obtained. MS (ESI) m/z=533.3 [M+H]⁺. ¹H NMR (400 MHz, DMSO-d6) δ 8.86-8.94 (m, 1H) 8.31-8.39 (m, 1H) 7.45 (br s, 1H) 7.17-7.31 (m, 3H) 6.71-6.77 (m, 1H) 4.37-4.47 (m, 1H) 4.10-4.18 (m, 3H) 3.50-3.56 (m, 2H) 3.08-3.16 (m, 4H) 2.91-3.00 (m, 2H) 2.77-2.82 (m, 2H) 2.51-2.56 (m, 4H) 2.37-2.45 (m, 2H).

### Example 18: Synthesis of Compound 21

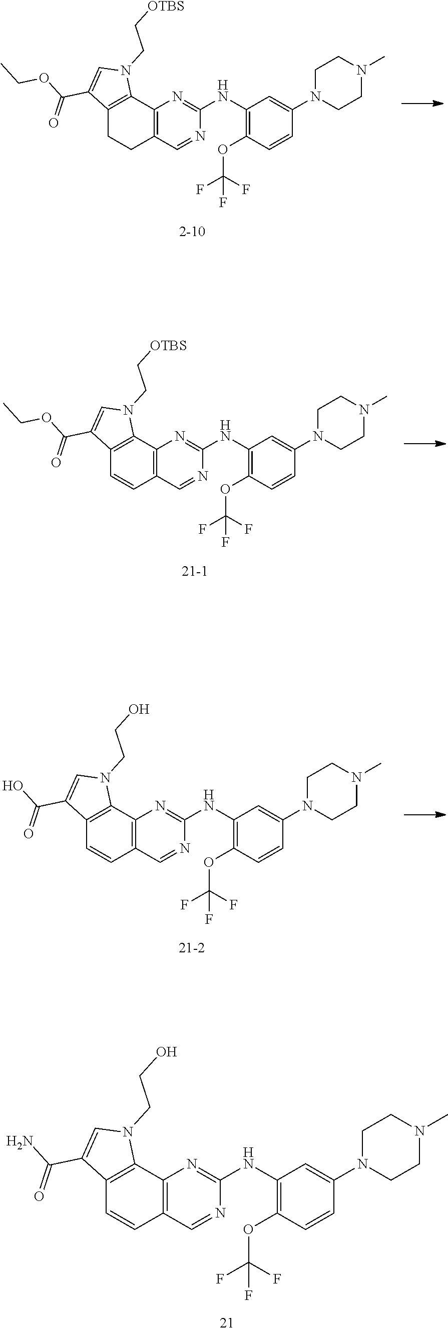

### 18.1 Synthesis of Intermediate 21-1

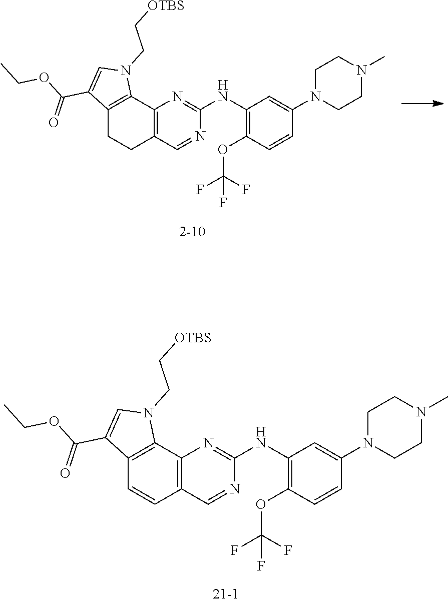

Intermediate 2-10 (100 mg, 148 μmol) and DDQ (168 mg, 741 μmol) were dissolved in dioxane (20 mL) and the mixture was reacted at 100° C. for 12 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (Waters Torus 2-PIC 150*19 mm*5 μm; mobile phase: [heptane-ethanol (0.1% NH₃H₂O)]; B %: 0%-20%, 15 min). The intermediate 21-1 (28 mg, yield 28.5%, yellow solid) was obtained. MS (ESI) m/z=673.3 [M+H]⁺.

### 18.2 Synthesis of Intermediate 21-2

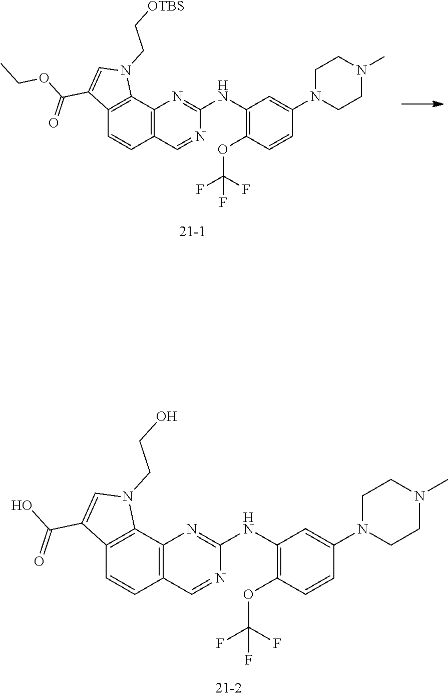 [SMILES: CCOC(=O)c1cn(CCO[Si](C)(C)C(C)(C)C)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)cn(CCO)c5c4n3)c2)CC1]

Intermediate 21-1 (40.0 mg, 59.4 μmol) was dissolved in tetrahydrofuran (2 mL) and methanol (2 mL), and a solution of lithium hydroxide hydrate (24.9 mg, 594 μmol) in water (2 mL) was added. The mixture was reacted at room temperature for 24 h, and then reacted at 50° C. for 24 h. The reaction liquid was concentrated under reduced pressure, the residue was slurried with petroleum ether and filtered, and the filter cake was collected and dried. Intermediate 21-2 (35 mg, crude product) was obtained. MS (ESI) m/z=531.2 [M+H]⁺.

### 18.3 Synthesis of Compound 21

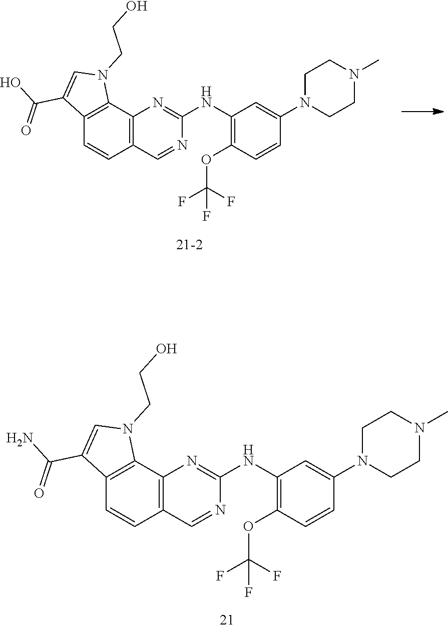 [SMILES: CN1CCN(c2ccc(OC(F)(F)F)c(Nc3ncc4ccc5c(C(=O)O)cn(CCO)c5c4n3)c2)CC1 ; CN1CCN(c2ccc(OC(F)(F)F)c(Nc3ncc4ccc5c(C(N)=O)cn(CCO)c5c4n3)c2)CC1]

Intermediate 21-2 (31.0 mg, 58.4 μmol), HATU (44.4 mg, 117 μmol) and DIEA (75.5 mg, 584 μmol) were dissolved in DMF (2 mL), and ammonium chloride (31.2 mg, 584 μmol) was added. The mixture was reacted at room temperature for 16 h. The reaction liquid was concentrated under reduced pressure, and the residue was purified by preparative liquid chromatogram (C18 150*30 mm*5 μm; mobile phase: [water (NH₄HCO₃)-acetonitrile]; B %: 27%-62%, 14 min) to obtain compound 21 (10 mg, yield 32%, white solid). MS (ESI) m/z=530.2 [M+H]⁺. ¹H NMR (DMSO-d₆) δ 9.18 (s, 1H), 9.13 (s, 1H), 8.16 (d, J=8.4 Hz, 1H), 8.06 (s, 1H), 7.50-7.48 (m, 2H), 7.26 (d, J=8.8 Hz, 1H), 7.17 (d, J=2.8 Hz, 1H), 6.87-6.84 (m, 2H), 4.66 (t, J=5.2 Hz, 1H), 4.60-4.58 (m, 2H), 3.42-3.41 (m, 2H), 3.19-3.17 (m, 4H), 2.50-2.48 (m, 4H), 2.25 (s, 3H).

### Example 19: Synthesis of Compound 22

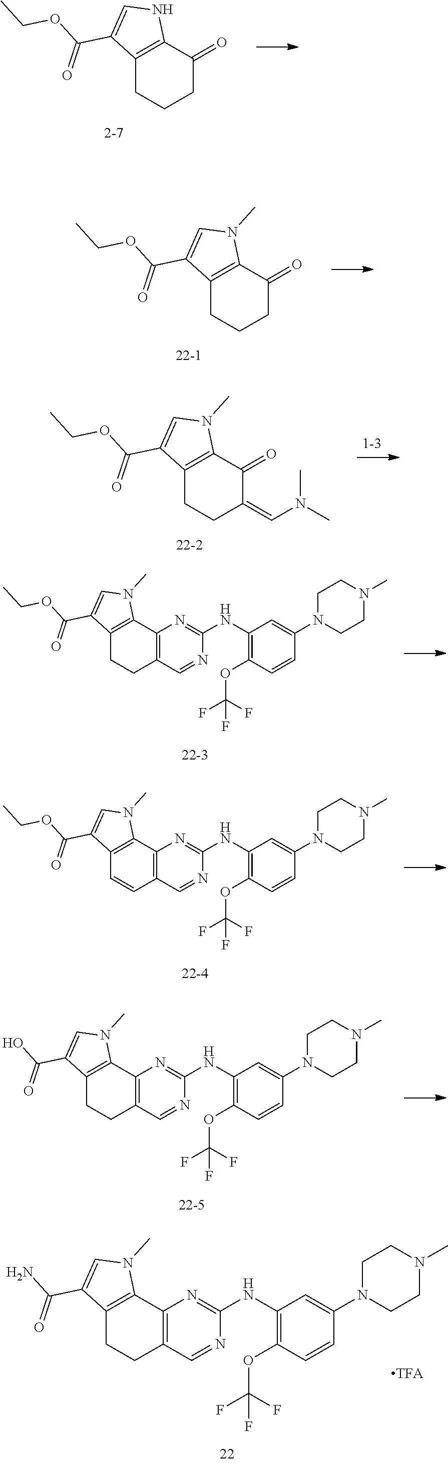

### 19.1 Synthesis of Intermediate 22-1

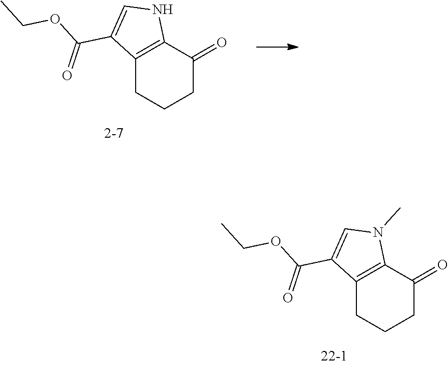 [SMILES: CCOC(=O)c1c[nH]c2c1CCCC2=O ; CCOC(=O)c1cn(C)c2c1CCCC2=O]

Intermediate 2-7 (0.44 g, 2.12 mmol) was dissolved in DMF (5 mL), K₂CO₃ (0.67 g, 4.83 mmol) and iodomethane (0.68 g, 4.83 mmol) were added and the mixture was reacted at room temperature for 4 h. Water (50 mL) was added to quench the reaction, and dichloromethane (50 mL×3) was added for extraction. The organic phases were combined, dried over anhydrous sodium sulfate, filtered, and the organic phase was concentrated under reduced pressure. The intermediate 22-1 (0.57 g, crude product) was obtained and used directly in the next step. MS (ESI) m/z=222.2 [M+H]⁺.

### 19.2 Synthesis of Intermediate 22-2

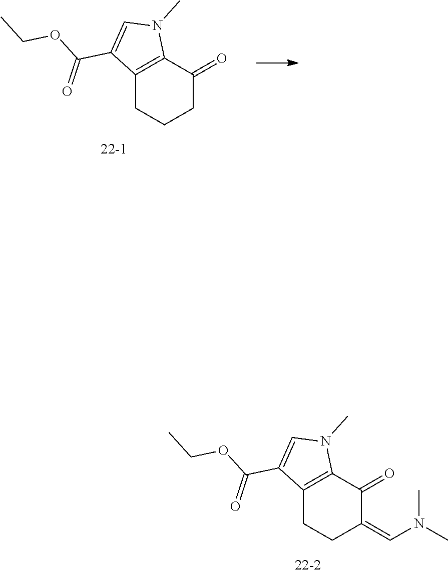 [SMILES: CCOC(=O)c1cn(C)c2c1CCCC2=O ; CCOC(=O)c1cn(C)c2c1CC/C(=C/N(C)C)C2=O]

Intermediate 22-1 (0.13 g, 0.588 mmol) was dissolved in DMF (5 mL), N,N-dimethylformamide dimethyl acetal (1.02 g, 8.58 mmol) was added, and the mixture was reacted under microwave at 145° C. for 6 h. The mixture was cooled to room temperature, water (50 mL) was added to quench the reaction, and ethyl acetate (50 mL×3) was added for extraction. The organic phases were combined, dried over anhydrous sodium sulfate, filtered, and the organic phase was concentrated under reduced pressure. The intermediate 22-2 (0.11 g, crude product) was obtained, which was used directly in the next step.

### 19.3 Synthesis of Intermediate 22-3

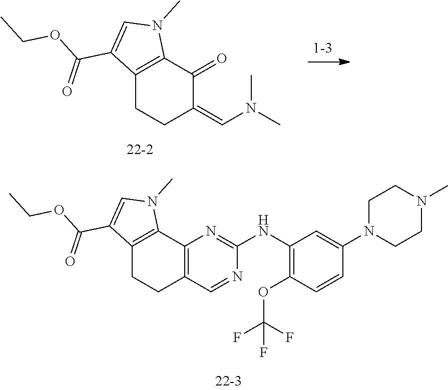

Intermediate 22-2 (0.11 g, 0.398 mmol) and intermediate 1-3 (0.19 g, 0.597 mmol) was dissolved in DMF (5 mL). The mixture was reacted at 110° C. for 16 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (methanol/dichloromethane=0%-8%) to obtain intermediate 22-3 (0.046 g, yield 21.6%, brown solid). MS (ESI) m/z=531.2 [M+H]⁺.

### 19.4 Synthesis of Intermediate 22-4

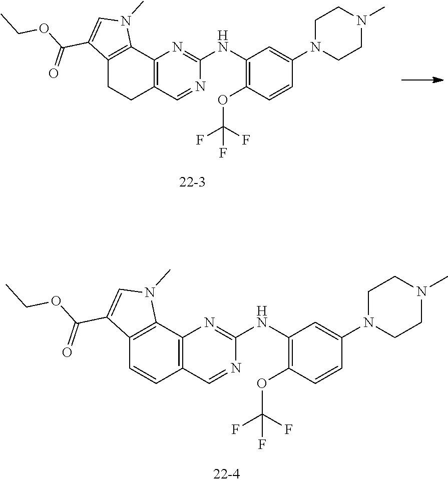

Intermediate 22-3 (60 mg, 113 μmol) and DDQ (128 mg, 565 μmol) were dissolved in dioxane (12 mL) and the mixture was reacted at 100° C. for 2 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (C18 150*19 mm*5 μm; mobile phase: [heptane-ethanol (0.1% NH₃H₂O)]; B %: 0%-20%, 15 min). The intermediate 22-4 (22 mg, yield 36.6%, yellow solid) was obtained. MS (ESI) m/z=529.2 [M+H]⁺.

### 19.5 Synthesis of Intermediate 22-5

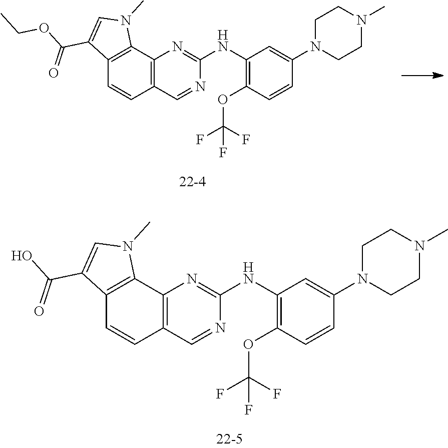 [SMILES: CCOC(=O)c1cn(C)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)cn(C)c5c4n3)c2)CC1]

Intermediate 22-4 (30.0 mg, 56.7 μmol) was dissolved in tetrahydrofuran (2 mL) and methanol (2 mL), and a solution of lithium hydroxide hydrate (23.8 mg, 567 μmol) in water (2 mL) was added. The mixture was reacted at room temperature for 24 h, then reacted at 50° C. for 24 h. The reaction liquid was concentrated under reduced pressure to obtain intermediate 22-5 (30 mg, crude product), which was directly used in the next step. MS (ESI) m/z=501.2 [M+H]⁺.

### 19.6 Synthesis of Compound 22

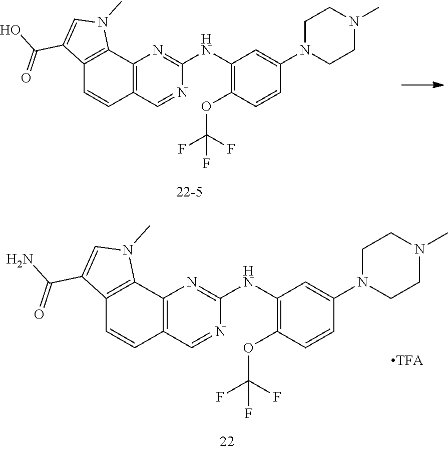 [SMILES: CN1CCN(c2ccc(OC(F)(F)F)c(Nc3ncc4ccc5c(C(=O)O)cn(C)c5c4n3)c2)CC1 ; CN1CCN(c2ccc(OC(F)(F)F)c(Nc3ncc4ccc5c(C(N)=O)cn(C)c5c4n3)c2)CC1]

Intermediate 22-5 (28.0 mg, 55.9 μmol), HATU (31.9 mg, 83.9 μmol) and DIEA (72.3 mg, 559 μmol) were dissolved in DMF (2 mL), and ammonium chloride (29.9 mg, 559 μmol) was added. The mixture was reacted at room temperature for 16 h. The reaction liquid was concentrated under reduced pressure, and the residue was purified by preparative liquid chromatogram (C18 150*30 mm*5 μm; mobile phase: [water (TFA)-acetonitrile]; B %: 20%-55%, 25 min) to obtain the compound 22 (5.00 mg, yield 14.6%, trifluoroacetate, yellow solid). MS (ESI) m/z=500.2 [M+H]⁺. ¹H NMR (400 MHz CD₃OD) δ 9.32 (s, 1H), 8.32 (d, J=8.8 Hz, 1H), 8.13 (d, J=5.2 Hz, 1H), 7.65 (d, J=8.8 Hz, 1H), 7.59 (d, J=3.2 Hz, 1H), 7.40 (d, J=9.2 Hz, 1H), 7.08-7.05 (m, 1H), 4.24 (s, 3H), 3.93 (d, J=13.2 Hz, 2H), 3.64 (d, J=11.6 Hz, 2H), 3.35-3.25 (m, 2H), 3.24-3.17 (m, 2H), 2.99 (s, 3H).

### Example 20: Synthesis of Compound 23

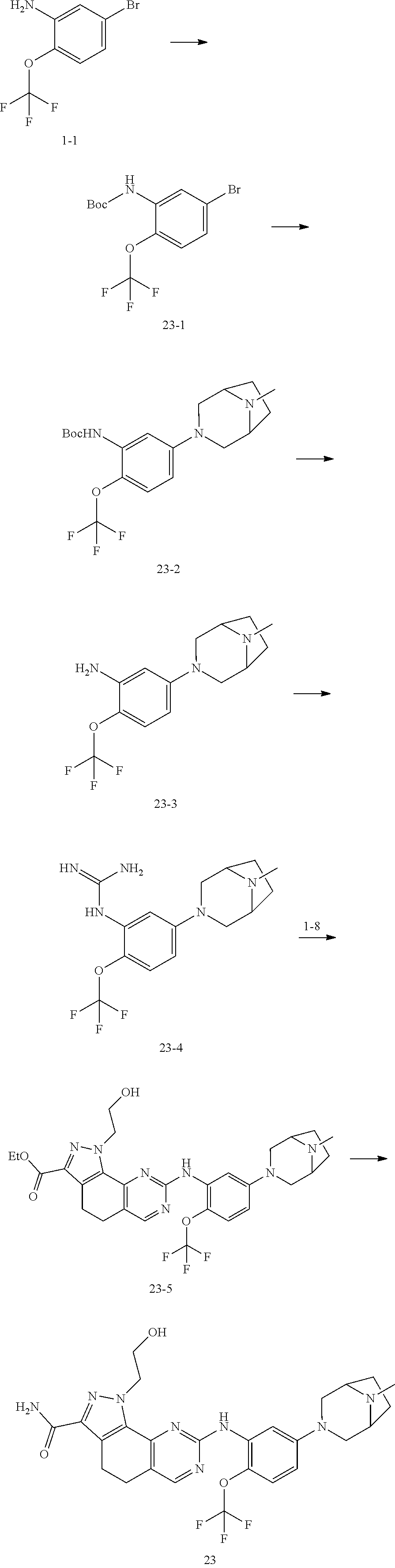

### 20.1 Synthesis of Intermediate 23-1

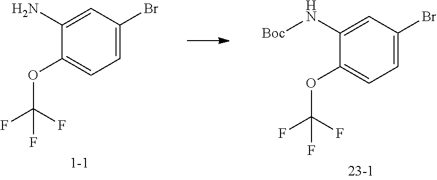 [SMILES: Nc1cc(Br)ccc1OC(F)(F)F ; CC(C)(C)OC(=O)Nc1cc(Br)ccc1OC(F)(F)F]

Intermediate 1-1 (2.0 g, 7.81 mmol) was dissolved in tetrahydrofuran (50 mL), KHMDS (1.0 M solution in tetrahydrofuran, 15.6 mL, 15.6 mmol) was added at 0° C. under nitrogen protection, and the mixture was reacted at 0° C. for 0.5 h. Then Boc₂O (1.7 g, 7.81 mol) was added at 0° C. under nitrogen protection, and the reaction was carried out at room temperature for 2 h. Water (20 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=10%-20%) to obtain intermediate 23-1 (2.2 g, yield 79%, yellow oil). MS (ESI) m/z=356.0 [M+H]⁺.

### 20.2 Synthesis of Intermediate 23-2

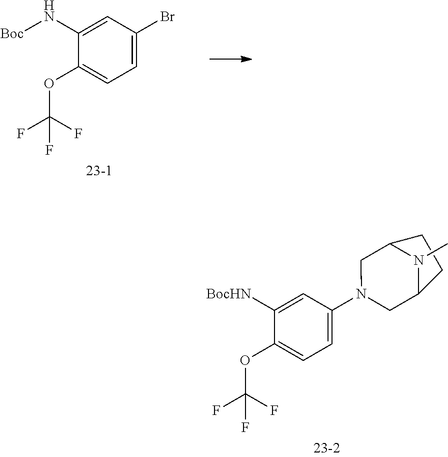 [SMILES: CC(C)(C)OC(=O)Nc1cc(Br)ccc1OC(F)(F)F ; CN1C2CCC1CN(c1ccc(OC(F)(F)F)c(NC(=O)OC(C)(C)C)c1)C2]

The intermediate 23-1 (2.2 g, 6.18 mmol), Cs₂CO₃ (9.07 g, 27.8 mmol), Pd₂(dba)₃ (286.2 mg, 0.6 mmol) and X-Phos (274.5 mg, 0.3 mmol) were dissolved in dioxane (100 mL), and 8-methyl-3,8-diazabicyclo[3.2.1]octane hydrochloride (1.23 g, 6.18 mmol) was added under nitrogen protection. The mixture was reacted at 100° C. overnight. Water (200 mL) was added to quench the reaction, and ethyl acetate (100 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=30%-50%) to obtain intermediate 23-2 (1.87 g, yield 75%, white solid). MS (ESI) m/z=402.1 [M+H]⁺.

### 20.3 Synthesis of Intermediate 23-3

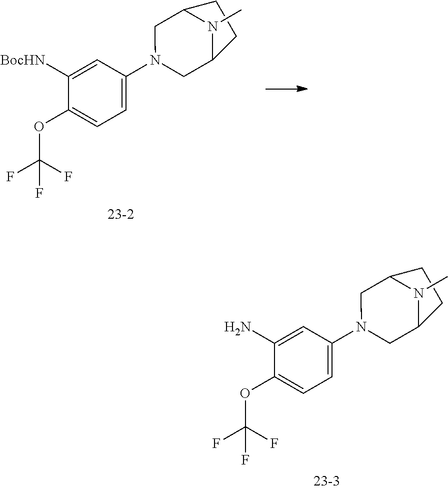 [SMILES: CN1C2CCC1CN(c1ccc(OC(F)(F)F)c(NC(=O)OC(C)(C)C)c1)C2 ; CN1C2CCC1CN(c1ccc(OC(F)(F)F)c(N)c1)C2]

Intermediate 23-2 (1.87 g, 4.66 mmol) was dissolved in hydrochloric acid/dioxane (20 mL) and the mixture was reacted at room temperature for 2 h. The reaction liquid was concentrated under reduced pressure. The residue was subjected to silica gel column chromatography (methanol/dichloromethane=5%-10%) to obtain intermediate 23-3 (0.73 g, yield 51.7%, yellow solid). MS (ESI) m/z=301.8[M+H]⁺.

### 20.4 Synthesis of Intermediate 23-4

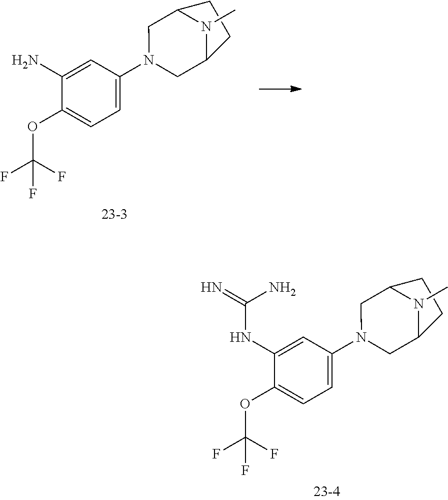 [SMILES: CN1C2CCC1CN(c1ccc(OC(F)(F)F)c(N)c1)C2 ; CN1C2CCC1CN(c1ccc(OC(F)(F)F)c(NC(=N)N)c1)C2]

Intermediate 23-3 (500 mg, 1.66 mmol), cyanamide (175 mg, 4.17 mmol) and TMSCl (254 mg, 3.54 mmol) were dissolved in acetonitrile (15 mL). The mixture was reacted under microwave at 140° C. for 1.5 h. The reaction liquid was concentrated under reduced pressure, and the residue was subjected to silica gel column chromatography (methanol/dichloromethane=5%-10%) to obtain intermediate 23-4 (530 mg, yield 94%, yellow solid). MS (ESI) m/z=344.0[M+H]⁺.

### 20.5 Synthesis of Intermediate 23-5

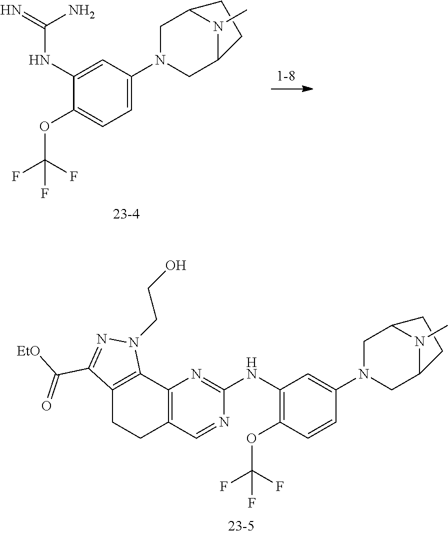 [SMILES: CN1C2CCC1CN(c1ccc(OC(F)(F)F)c(NC(=N)N)c1)C2 ; CCOC(=O)c1nn(CCO)c2c1CCc1cnc(Nc3cc(N4CC5CCC(C4)N5C)ccc3OC(F)(F)F)nc1-2]

Intermediate 1-8 (475 mg, 1.56 mmol) and intermediate 23-4 (530 mg, 1.56 mmol) were dissolved in DMF (10 mL), and the mixture was reacted at 110° C. for 16 h. The mixture was diluted with ethyl acetate (100 mL), and extracted with water (50 mL×3). The organic phases were collected, dried over anhydrous sodium sulfate, filtered, and the organic phase was concentrated under reduced pressure. The residue was subjected to silica gel column chromatography (methanol/dichloromethane=10%-13%) to obtain intermediate 23-5 (240 mg, yield 26.2%, white solid). MS (ESI) m/z=588.0 [M+H]⁺.

### 20.6 Synthesis of Compound 23

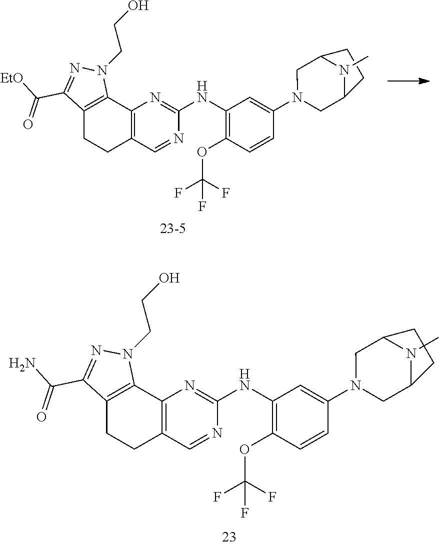 [SMILES: CCOC(=O)c1nn(CCO)c2c1CCc1cnc(Nc3cc(N4CC5CCC(C4)N5C)ccc3OC(F)(F)F)nc1-2 ; CN1C2CCC1CN(c1ccc(OC(F)(F)F)c(Nc3ncc4c(n3)-c3c(c(C(N)=O)nn3CCO)CC4)c1)C2]

Intermediate 23-5 (200 mg, 0.340 mmol) was dissolved in NH₃/MeOH (10.0 mL). The mixture was reacted under microwave at 110° C. for 2 h. The reaction liquid was concentrated under reduced pressure, and the residue was purified by preparative liquid chromatogram (C18 30*150 mm*7 μm; mobile phase: [water-acetonitrile]; B %: 5%-30%, 40 min). Compound 23 (56.7 mg, yield 37.5%, yellow solid) was obtained. MS (ESI) m/z=559.0 [M+H]⁺. ¹H NMR (400 MHz, DMSO-d6) δ 8.82 (s, 1H), 8.32 (s, 1H), 7.43 (s, 1H), 7.24 (s, 1H), 7.17 (dd, J=1.2 Hz, 9.2 Hz, 1H), 7.06 (d, J=2.8 Hz, 1H), 6.49 (dd, J=3.2 Hz, 9.2 Hz, 1H), 4.62-4.58 (m, 3H), 3.61 (d, J=5.2 Hz, 2H), 3.31 (s, 2H), 3.19 (s, 2H), 2.97 (t, J=7.6 Hz, 2H), 2.85-2.77 (m, 4H), 2.22 (s, 3H), 1.95-1.93 (m, 2H), 1.61 (d, J=7.6 Hz, 2H).

### Example 21: Synthesis of Compound 24

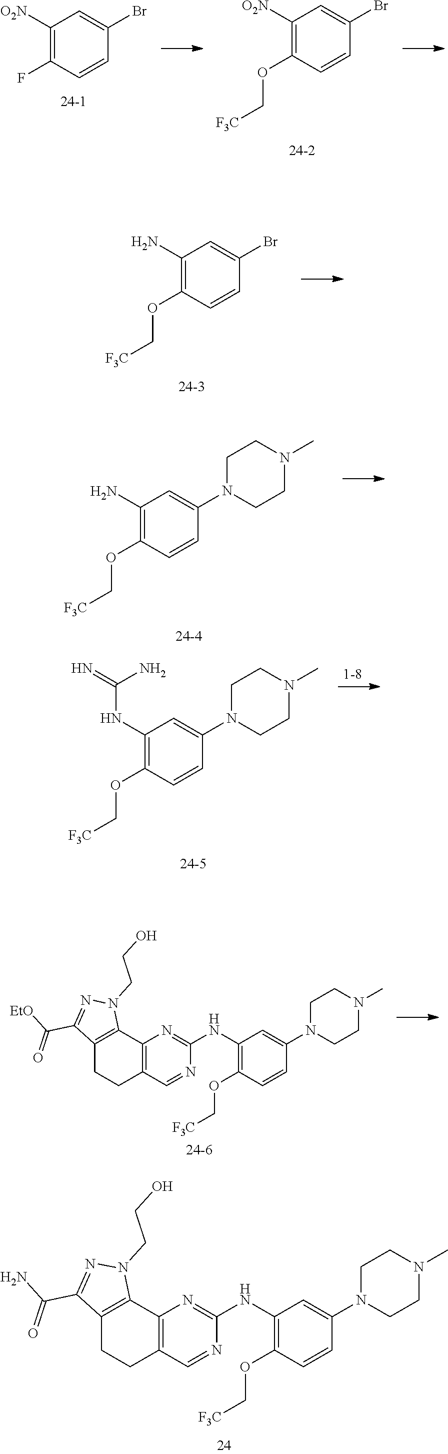

### 21.1 Synthesis of Intermediate 24-2

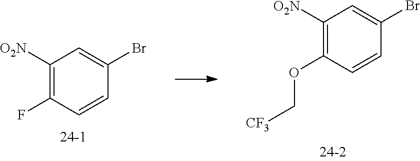 [SMILES: O=[N+]([O-])c1cc(Br)ccc1F ; O=[N+]([O-])c1cc(Br)ccc1OCC(F)(F)F]

Intermediate 24-1 (7.0 g, 31.8 mmol), trifluoroethanol (31.8 g, 318 mmol) and cesium carbonate (25.9 g, 79.5 mmol) were reacted at 70° C. for 4 h. The reaction liquid was filtered, and the filtrate was dissolved in ethyl acetate (200 mL), and the obtained product was washed with water (200 mL). The organic phases were collected, dried over anhydrous sodium sulfate, filtered, and the organic phase was concentrated under reduced pressure. The intermediate 24-2 (7.8 g, yield 80%, yellow oil) was obtained. MS (ESI) m/z=299.9 [M+H]⁺.

### 21.2 Synthesis of Intermediate 24-3

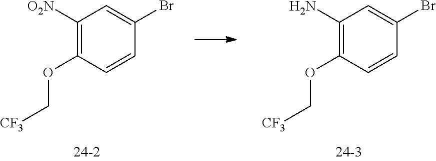 [SMILES: O=[N+]([O-])c1cc(Br)ccc1OCC(F)(F)F ; Nc1cc(Br)ccc1OCC(F)(F)F]

Intermediate 24-2 (5.1 g, 17.0 mmol) was dissolved in ethanol/acetic acid (200 mL/200 mL), iron powder (9.49 g, 170 mmol) was added, and the mixture was reacted at 80° C. for 2 h. The reaction liquid was filtered, and the filtrate was concentrated under reduced pressure. The residue was dissolved in ethyl acetate (200 mL), and the obtained product was washed with saturated sodium bicarbonate (200 mL) and water (200 mL). The organic phases were collected, dried over anhydrous sodium sulfate, filtered, and the organic phase was concentrated under reduced pressure. The intermediate 24-3 (3.2 g, yield 68.5%, yellow solid) was obtained. MS (ESI) m/z=270.0 [M+H]⁺.

### 21.3 Synthesis of Intermediate 24-4

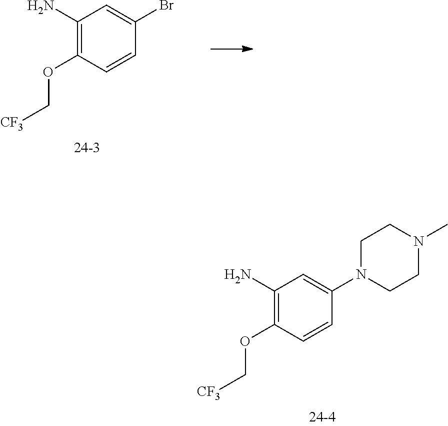 [SMILES: Nc1cc(Br)ccc1OCC(F)(F)F ; CN1CCN(c2ccc(OCC(F)(F)F)c(N)c2)CC1]

Intermediate 24-3 (3.0 g, 11.1 mmol), K₃PO₄ (4.71 g, 22.2 mmol), BFMO (0.29 g, 0.6 mmol), CuI (0.21 g, 1.1 mmol), BFMO (0.28 g, 1.1 mmol) and N-methylpiperazine (3.34 g, 33.3 mmol) were dissolved in ethanol (50 mL). The mixture was reacted at 100° C. for 24 h under nitrogen protection. The reaction liquid was cooled to room temperature, dissolved in dichloromethane (50 mL), filtered, and the organic phase was collected. The organic phase was dried over anhydrous sodium sulfate, filtered, and the organic phase was concentrated under reduced pressure. The residue was subjected to silica gel column chromatography (ethyl acetate/petroleum ether=10%-50%) to obtain intermediate 24-4 (1.2 g, yield 36.9%, white solid). MS (ESI) m/z=290.2 [M+H]⁺.

### 21.4 Synthesis of Intermediate 24-5

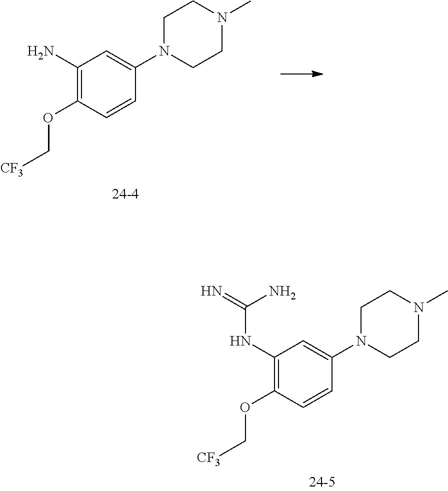 [SMILES: CN1CCN(c2ccc(OCC(F)(F)F)c(N)c2)CC1 ; CN1CCN(c2ccc(OCC(F)(F)F)c(NC(=N)N)c2)CC1]

Intermediate 24-4 (1.1 g, 3.8 mmol), cyanamide (0.32 g, 7.6 mmol) and TMSCl (0.62 g, 5.7 mmol) were dissolved in acetonitrile (15 mL). The mixture was reacted under microwave at 140° C. for 2 h. The reaction liquid was concentrated under reduced pressure, and the residue was subjected to column chromatography (C18, ACN/H₂O=3/97-25/75) to obtain intermediate 24-5 (1.0 g, yield 78.9%, yellow solid). MS (ESI) m/z=332.0[M+H]⁺.

### 21.5 Synthesis of Intermediate 24-6

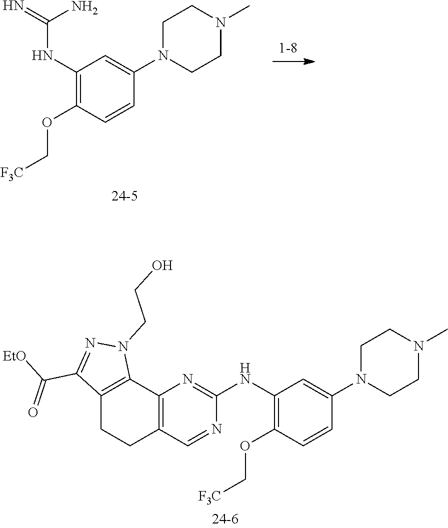 [SMILES: CN1CCN(c2ccc(OCC(F)(F)F)c(NC(=N)N)c2)CC1 ; CCOC(=O)c1nn(CCO)c2c1CCc1cnc(Nc3cc(N4CCN(C)CC4)ccc3OCC(F)(F)F)nc1-2]

Intermediate 1-8 (1.06 g, 3.44 mmol) and intermediate 24-5 (0.95 g, 2.86 mmol) were dissolved in DMF (60 mL) and the mixture was reacted at 110° C. for 3 h. The reaction liquid was concentrated under reduced pressure, and the residue was subjected to column chromatography (C18, ACN/H₂O=5/95-80/20) to obtain intermediate 24-6 (0.40 g, yield 24.5%, yellow solid). MS (ESI) m/z=576.0[M+H]⁺.

### 21.6 Synthesis of Compound 24

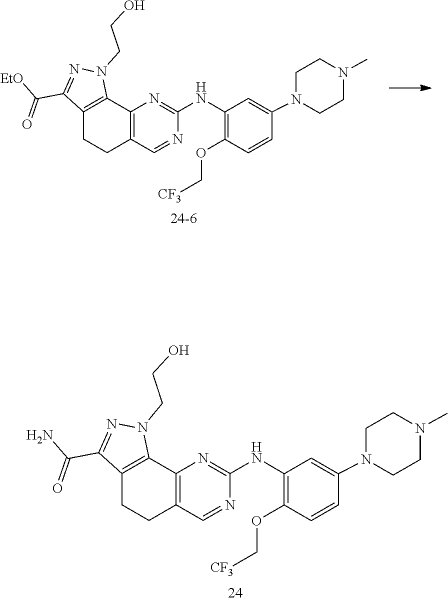 [SMILES: CCOC(=O)c1nn(CCO)c2c1CCc1cnc(Nc3cc(N4CCN(C)CC4)ccc3OCC(F)(F)F)nc1-2 ; CN1CCN(c2ccc(OCC(F)(F)F)c(Nc3ncc4c(n3)-c3c(c(C(N)=O)nn3CCO)CC4)c2)CC1]

Intermediate 24-6 (300 mg, 0.52 mmol) was dissolved in NH₃/MeOH (10.0 mL). The mixture was reacted for 17 h under sealed conditions at 120° C. The reaction liquid was concentrated under reduced pressure, and the residue was purified by preparative liquid chromatogram (C18 30*150 mm*7 μm; mobile phase: [water-acetonitrile]; B %: 5%-30%, 40 min). Compound 24 (105 mg, yield 36%, yellow solid) was obtained. MS (ESI) m/z=547.1 [M+H]⁺. ¹H NMR (400 MHz, DMSO-d6) δ 8.37 (s, 1H), 8.18 (s, 1H), 7.50-7.47 (m, 2H), 7.27 (s, 1H), 7.06 (d, J=8.8 Hz, 1H), 6.66 (d, J=8.8, 2.8 Hz, 1H), 4.70-4.63 (m, 5H), 3.72-3.70 (m, 2H), 3.06-3.04 (m, 4H), 2.97 (t, J=7.6 Hz, 2H), 2.79 (t, J=7.6 Hz, 2H), 2.50-2.44 (m, 4H), 2.22 (s, 3H).

### Example 22: Synthesis of Compound 25

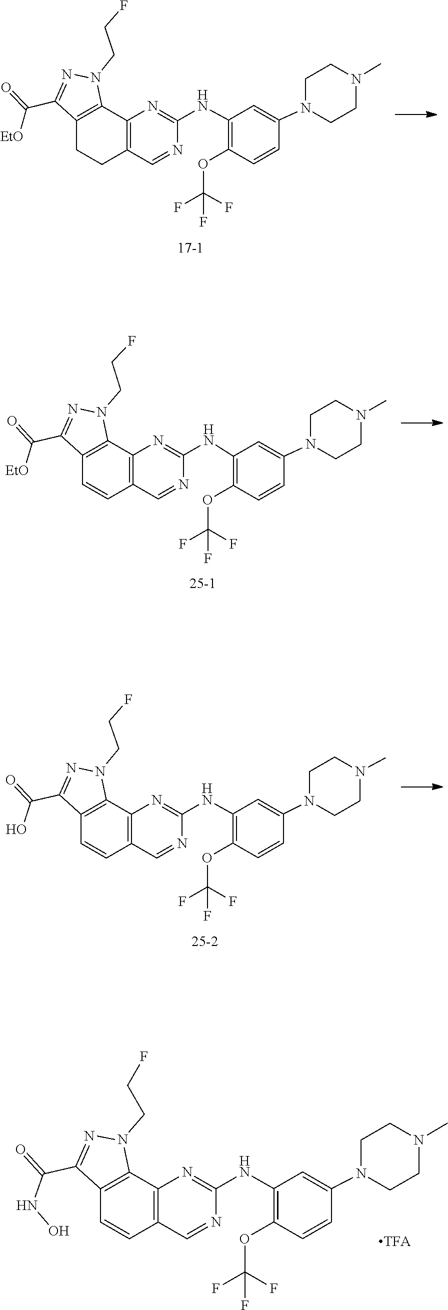 [SMILES: CCOC(=O)c1nn(CCF)c2c1CCc1cnc(Nc3cc(N4CCN(C)CC4)ccc3OC(F)(F)F)nc1-2 ; CCOC(=O)c1nn(CCF)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)nn(CCF)c5c4n3)c2)CC1 ; CN1CCN(c2ccc(OC(F)(F)F)c(Nc3ncc4ccc5c(C(=O)NO)nn(CCF)c5c4n3)c2)CC1]

### 22.1 Synthesis of Intermediate 25-1

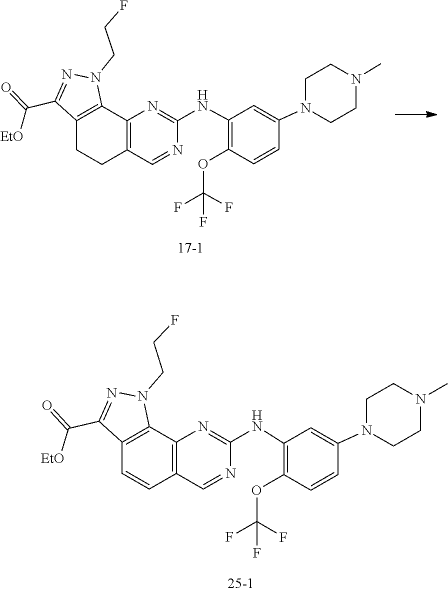 [SMILES: CCOC(=O)c1nn(CCF)c2c1CCc1cnc(Nc3cc(N4CCN(C)CC4)ccc3OC(F)(F)F)nc1-2 ; CCOC(=O)c1nn(CCF)c2c1ccc1cnc(Nc3cc(N4CCN(C)CC4)ccc3OC(F)(F)F)nc12]

Intermediate 17-1 (160 mg, 0.280 mmol) and DDQ (319 mg, 1.41 mmol) were dissolved in dioxane (20 mL) and the mixture was reacted at 100° C. for 2 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (C18 150*40 mm*10 μm; mobile phase: [water (NH₄HCO₃)-ACN-acetonitrile]; B %: 45%-85%, 10 min). Intermediate 25-1 (57 mg, yield 35.7%, brown solid) was obtained. MS (ESI) m/z=562.2 [M+H]⁺.

### 22.2 Synthesis of Intermediate 25-2

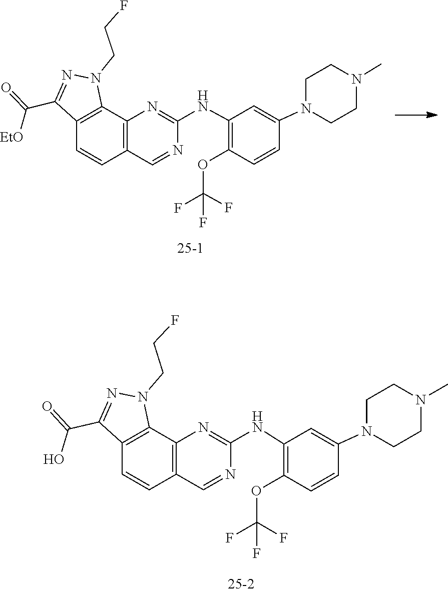 [SMILES: CCOC(=O)c1nn(CCF)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)nn(CCF)c5c4n3)c2)CC1]

Intermediate 25-1 (45.0 mg, 82.2 μmol) was dissolved in methanol (2 mL) and lithium hydroxide aqueous solution (1 M, 411 μL) was added. The mixture was reacted at room temperature for 6 h. The pH was adjusted to 5-7 with hydrochloric acid at 0° C., the residue was purified by preparative liquid chromatogram (C18 150*30 mm*5 μm; mobile phase: [water (TFA)-acetonitrile]; B %: 10%-50%, 10 min) to obtain intermediate 25-2 (14.8 mg, yield 30.1%, brown solid). MS (ESI) m/z=534.2 [M+H]⁺.

### 22.3 Synthesis of Compound 25

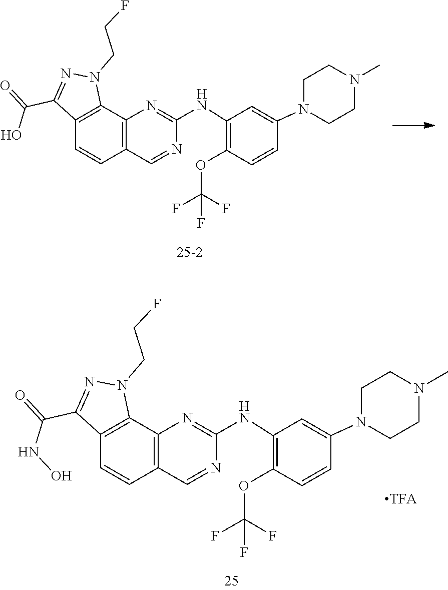 [SMILES: CN1CCN(c2ccc(OC(F)(F)F)c(Nc3ncc4ccc5c(C(=O)O)nn(CCF)c5c4n3)c2)CC1 ; CN1CCN(c2ccc(OC(F)(F)F)c(Nc3ncc4ccc5c(C(=O)NO)nn(CCF)c5c4n3)c2)CC1]

Intermediate 25-2 (20.9 mg, 39.3 μmol), HATU (16.4 mg, 43.2 μmol) and DIEA (10.2 mg, 78.67 μmol) were dissolved in DMF (2 mL), and hydroxylamine hydrochloride (10.2 mg, 78.67 μmol) was added. The mixture was reacted at room temperature for 2 h. The reaction liquid was concentrated under reduced pressure, and the residue was purified by preparative liquid chromatogram (C18 150*30 mm*5 μm; mobile phase: [water (TFA)-acetonitrile]; B %: 20%-50%, 25 min) to obtain the compound 25 (2.5 mg, yield 9.6%, brown solid). MS (ESI) m/z=549.2 [M+H]⁺. ¹H NMR (400 MHz, MeOD) δ 9.22 (s, 1H), 8.05 (d, J=8.76 Hz, 1H), 7.53-7.61 (m, 2H), 7.36 (d, J=8.88 Hz, 1H), 6.97 (dd, J=9.07, 2.81 Hz, 1H), 5.21-5.31 (m, 2H), 4.81 (d, J=4.88 Hz, 1H), 4.69 (t, J=4.88 Hz, 1H), 3.93 (d, J=13.51 Hz, 2H), 3.64 (d, J=12.01 Hz, 2H), 3.26-3.30 (m, 2H), 3.05-3.17 (m, 2H), 2.99 (s, 3H).

### Example 23: Synthesis of Compound 16

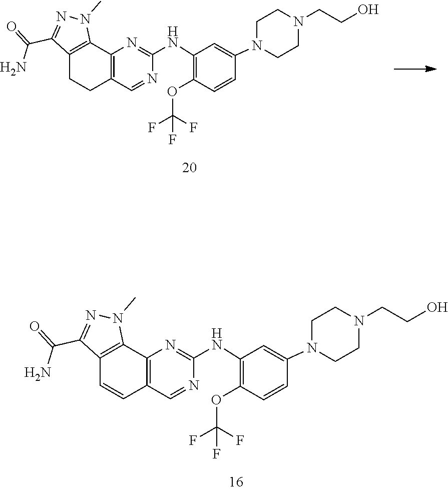 [SMILES: Cn1nc(C(N)=O)c2c1-c1nc(Nc3cc(N4CCN(CCO)CC4)ccc3OC(F)(F)F)ncc1CC2 ; Cn1nc(C(N)=O)c2ccc3cnc(Nc4cc(N5CCN(CCO)CC5)ccc4OC(F)(F)F)nc3c21]

Compound 20 (149 mg, 0.28 mmol) and DDQ (319 mg, 1.41 mmol) were dissolved in dioxane (20 mL) and the mixture was reacted at 100° C. for 2 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (C18 150*40 mm*10 μm; mobile phase: [water (NH₄HCO₃)-ACN-acetonitrile]; B %: 45%-85%, 10 min). Compound 16 (14.8 mg, yield 10%, brown solid) was obtained. MS (ESI) m/z=531.2 [M+H]⁺.

### Example 24: Synthesis of Compound 26

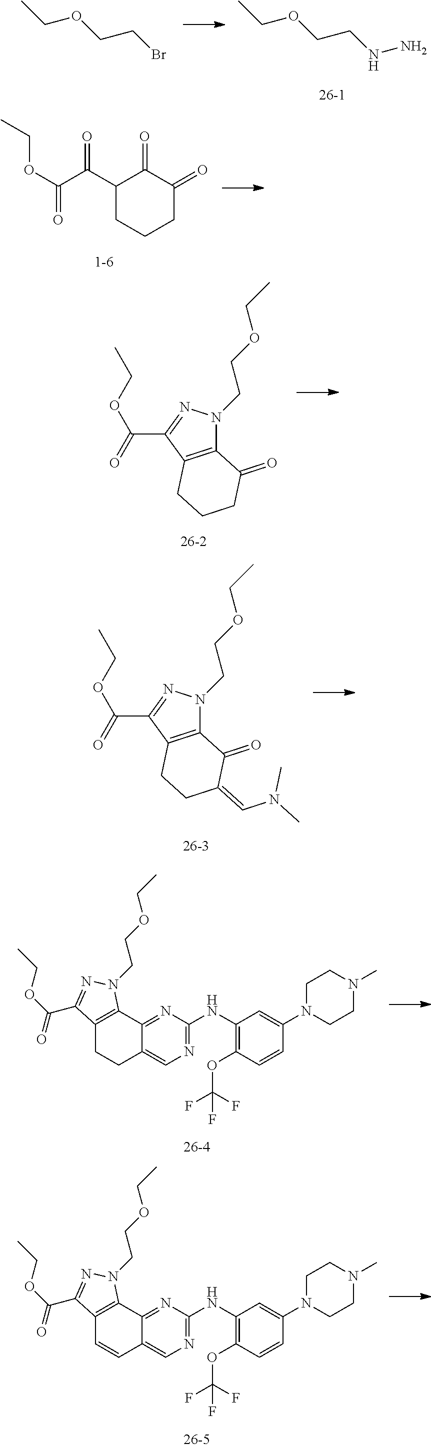

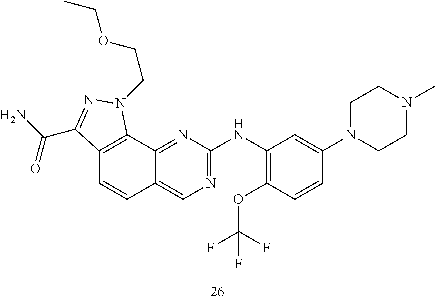 [SMILES: CCOCCn1nc(C(N)=O)c2ccc3cnc(Nc4cc(N5CCN(C)CC5)ccc4OC(F)(F)F)nc3c21]

### 24.1 Synthesis of Intermediate 26-1

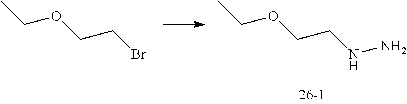

Compounds 2-bromoethyl ethyl ether (1.00 g, 6.54 mmol) and tert-butyl 2-(propane-2-ylidene)hydrazinecarboxylate (1.13 g, 6.54 mmol) were dissolved in acetonitrile (40 mL), and NaH (324 mg, 13.5 mmol) was added. The mixture was reacted at 80° C. for 12 h and detection showed that the reaction was completed. Water was added to quench the reaction, ethyl acetate (40 mL×3) was added for extraction, the obtained product was dried over anhydrous sodium sulfate, and filtered, the organic phase was collected, and concentrated under reduced pressure. The residue was dissolved in ethyl acetate hydrochloride (4 M, 10 mL) and the mixture was reacted at room temperature overnight. The reaction liquid was concentrated, the residue was dissolved in ethanol (20 mL), concentrated hydrochloric acid (5 mL) was added, and the mixture was stirred overnight. The reaction liquid was concentrated under reduced pressure to obtain intermediate 26-1 (0.95 g, crude product), which was directly used in the next step.

### 24.2 Synthesis of Intermediate 26-2

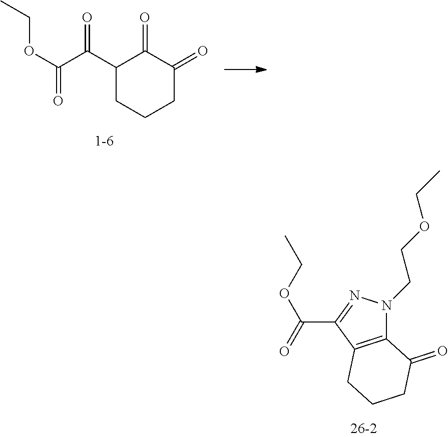 [SMILES: CCOC(=O)C(=O)C1CCCC(=O)C1=O ; CCOCCn1nc(C(=O)OCC)c2c1C(=O)CCC2]

Intermediate 1-6 (850 mg, 3.54 mmol) was dissolved in acetic acid (10 mL), compound 26-1 (737 mg, 7.08 mmol) was added, and the mixture was reacted at room temperature for 15 h. Water (20 mL) was added to quench the reaction, saturated Na₂CO₃ (100 mL) was added, and dichloromethane (80 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-5%) to obtain intermediate 26-2 (473 mg, yield 47.8%, yellow solid). MS (ESI) m/z=281.2 [M+H]⁺.

### 24.3 Synthesis of Intermediate 26-3

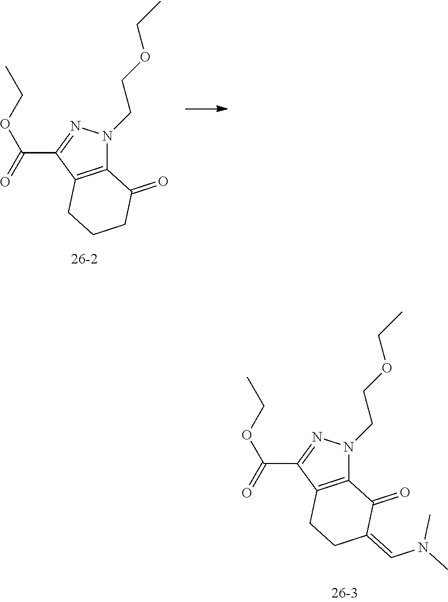 [SMILES: CCOCCn1nc(C(=O)OCC)c2c1C(=O)CCC2 ; CCOCCn1nc(C(=O)OCC)c2c1C(=O)/C(=C\N(C)C)CC2]

Intermediate 26-2 (600 mg, 2.14 mmol) was dissolved in DMF-DMA (10 mL) and the mixture was reacted at 110° C. for 12 h. The reaction liquid was concentrated under reduced pressure to obtain intermediate 26-3 (650 mg, crude product), which was directly used in the next step.

### 24.4 Synthesis of Intermediate 26-4

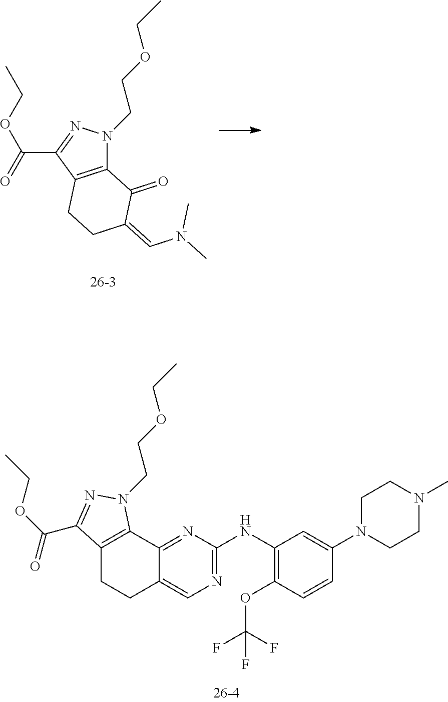 [SMILES: CCOCCn1nc(C(=O)OCC)c2c1C(=O)/C(=C\N(C)C)CC2 ; CCOCCn1nc(C(=O)OCC)c2c1-c1nc(Nc3cc(N4CCN(C)CC4)ccc3OC(F)(F)F)ncc1CC2]

Intermediate 26-3 (600 mg, 1.79 mmol) and intermediate 1-3 (851 mg, 2.68 mmol) were dissolved in DMF (20 mL) and the mixture was reacted at 110° C. for 12 h. The reaction liquid was concentrated under reduced pressure, and the residue was subjected to silica gel column chromatography (ethyl acetate/petroleum ether=0%-5%) to obtain intermediate 26-4 (228 mg, yield 21.6%, yellow solid). MS (ESI) m/z=590.3 [M+H]⁺.

### 24.5 Synthesis of Intermediate 26-5

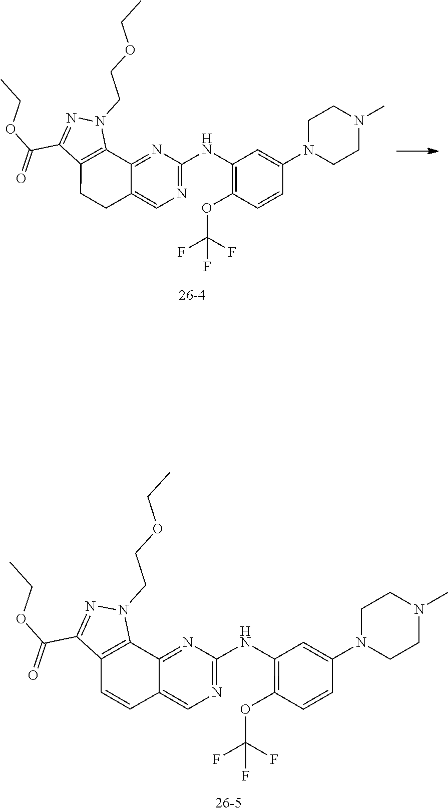

Intermediate 26-4 (44.0 mg, 74.6 μmol) and DDQ (88.0 mg, 387 μmol) were dissolved in dioxane (20 mL) and the mixture was reacted at 100° C. for 12 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatography (C18 150*40 mm*10 μm; mobile phase: [water (TFA)-acetonitrile]; B %: 44%-84%, 30 min). Intermediate 26-5 (16 mg, yield 36.5%, brown solid) was obtained. MS (ESI) m/z=588.4 [M+H]⁺.

### 24.6 Synthesis of Compound 26

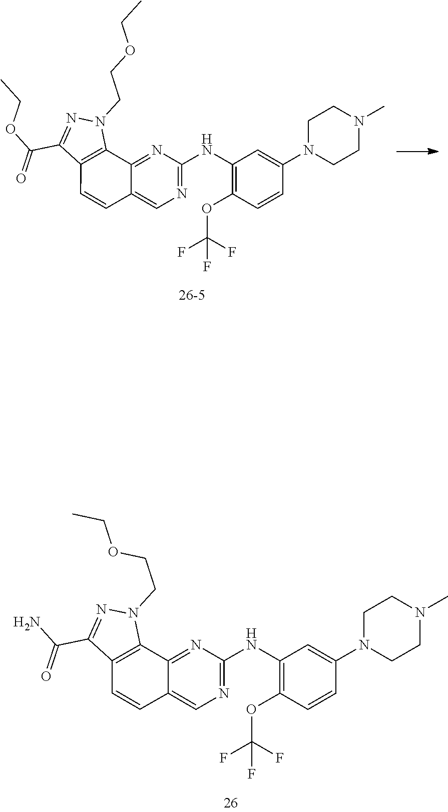 [SMILES: CCOCCn1nc(C(=O)OCC)c2ccc3cnc(Nc4cc(N5CCN(C)CC5)ccc4OC(F)(F)F)nc3c21 ; CCOCCn1nc(C(N)=O)c2ccc3cnc(Nc4cc(N5CCN(C)CC5)ccc4OC(F)(F)F)nc3c21]

Intermediate 26-5 (16 mg, 27.2 μmol) was dissolved in NMP (6 mL), and NH₃/MeOH (50.0 mL) was added. The mixture was reacted for 48 h under sealed conditions at 80.0° C. The reaction liquid was concentrated under reduced pressure, and the residue was purified by preparative liquid chromatogram (C18 30*150 mm*7 μm; mobile phase: [water (TFA)-acetonitrile]; B %: 10%-80%, 40 min). Compound 26 (56.7 mg, yield 37.5%, yellow solid) was obtained. MS (ESI) m/z=531.2 [M+H]⁺. H NMR (400 MHz, DMSO-d6) δ 9.59 (s, 1H), 9.38 (s, 1H), 8.05-8.07 (d, J=8.00 Hz, 1H), 7.82 (s, 1H), 7.63-7.66 (d, J=12.0 Hz, 1H), 7.53 (s, 1H), 7.28-7.36 (m, 2H), 6.93-6.95 (m, 1H), 5.03-5.06 (t, J=8.00 Hz, 2H), 3.67-3.70 (t, J=8.00 Hz, 2H), 3.17-3.25 (m, 6H), 2.56-2.57 (m, 3H), 2.28 (s, 3H), 1.28-1.37 (m, 1H), 0.88-0.91 (m, 3H).

### Example 25: Synthesis of Compound 27

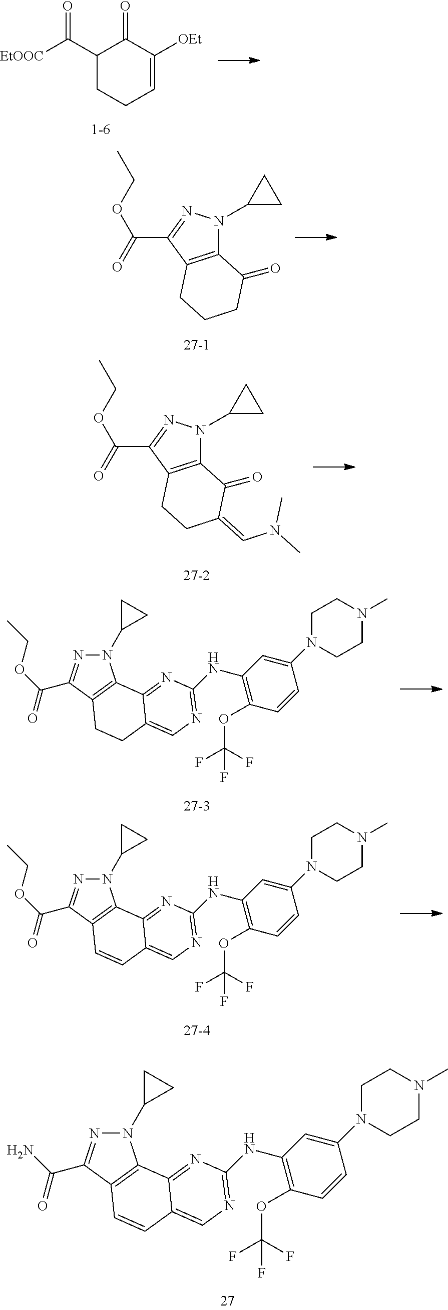

### 25.1 Synthesis of Intermediate 27-1

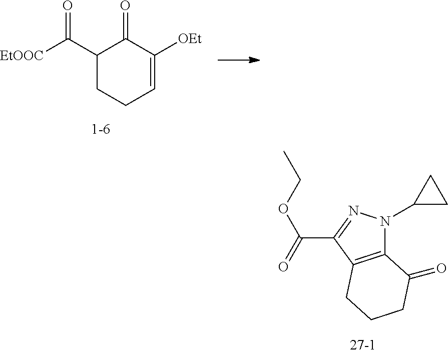 [SMILES: CCOC(=O)C(=O)C1CCC=C(OCC)C1=O ; CCOC(=O)c1nn(C2CC2)c2c1CCCC2=O]

Intermediate 1-6 (200 mg, 832 μmol) was dissolved in acetic acid (10 mL), compound cyclopropylhydrazine (71.9 mg, 998 μmol) was added, and the mixture was reacted at room temperature for 15 h. Water (20 mL) was added to quench the reaction, saturated Na₂CO₃ (100 mL) was added, and dichloromethane (80 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-10%) to obtain intermediate 27-1 (90 mg, yield 43.6%, yellow solid). MS (ESI) m/z=249.1 [M+H]⁺.

### 25.2 Synthesis of Intermediate 27-2

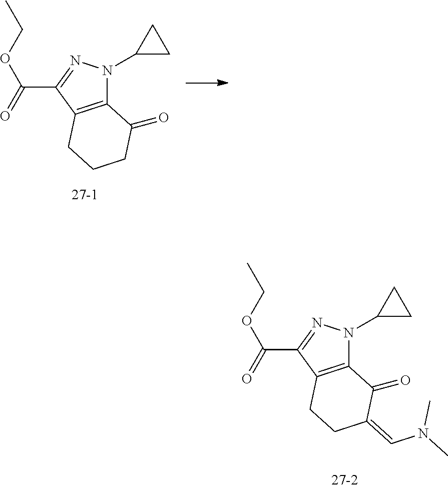 [SMILES: CCOC(=O)c1nn(C2CC2)c2c1CCCC2=O ; CCOC(=O)c1nn(C2CC2)c2c1CC/C(=C/N(C)C)C2=O]

Intermediate 27-1 (500 mg, 2.01 mmol) was dissolved in DMF-DMA (10 mL) and the mixture was reacted at 110° C. for 12 h. The reaction liquid was concentrated under reduced pressure, and the residue was dissolved in methyl tertiary ether and the mixture was stirred for 3 h. The resulting product was filtered, the filter cake was collected, and dried to obtain intermediate 27-2 (500 mg, crude product), which was directly used in the next step.

### 25.3 Synthesis of Intermediate 27-3

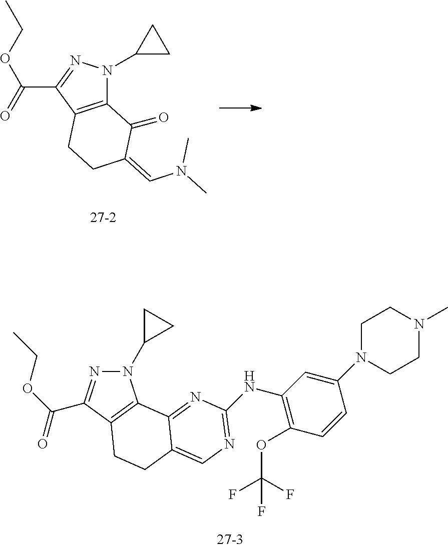 [SMILES: CCOC(=O)c1nn(C2CC2)c2c1CC/C(=C/N(C)C)C2=O ; CCOC(=O)c1nn(C2CC2)c2c1CCc1cnc(Nc3cc(N4CCN(C)CC4)ccc3OC(F)(F)F)nc1-2]

Intermediate 27-2 (500 mg, 1.65 mmol) and intermediate 1-3 (630 mg, 1.98 mmol) were dissolved in DMF (20 mL) and the mixture was reacted at 110° C. for 12 h. The reaction liquid was concentrated under reduced pressure, and the residue was subjected to silica gel column chromatography (ethyl acetate/petroleum ether=0%-5%) to obtain intermediate 27-3 (100 mg, yield 10.9%, yellow solid). MS (ESI) m/z=558.2 [M+H]⁺.

### 25.4 Synthesis of Intermediate 27-4

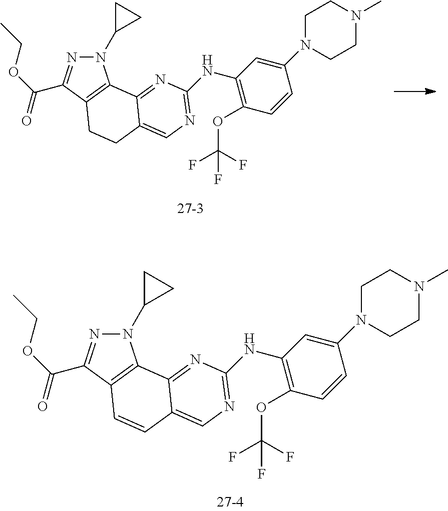

Intermediate 27-3 (50.0 mg, 89.7 μmol) and DDQ (101 mg, 444 μmol) were dissolved in dioxane (20 mL) and the mixture was reacted at 100° C. for 12 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (C18 150*40 mm*10 μm; mobile phase: [water (TFA)-acetonitrile]; B %: 51%-81%, 10 min). Intermediate 27-4 (30 mg, yield 20.1%, brown solid) was obtained. MS (ESI) m/z=556.3 [M+H]⁺.

### 25.5 Synthesis of Compound 27

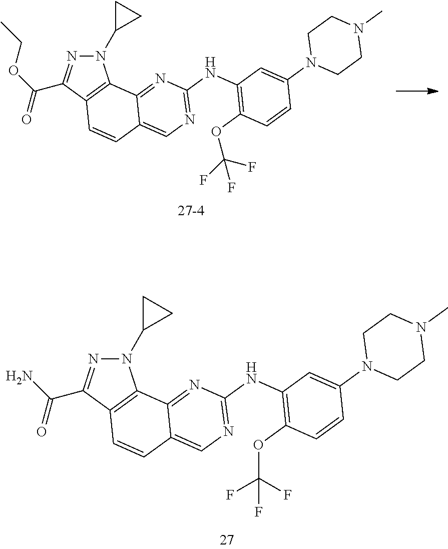 [SMILES: CCOC(=O)c1nn(C2CC2)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(N)=O)nn(C6CC6)c5c4n3)c2)CC1]

Intermediate 27-4 (30.0 mg, 54.0 μmol) was dissolved in NMP (6 mL), and NH₃/MeOH (50.0 mL) was added. The mixture was reacted for 48 h under sealed conditions at 80.0° C. The reaction liquid was concentrated under reduced pressure, and the residue was purified by preparative liquid chromatogram (C18 30*150 mm*7 μm; mobile phase: [water (TFA)-acetonitrile]; B %: 40%-80%, 14 min). Compound 27 (17.0 mg, yield 59.8%, yellow solid) was obtained. MS (ESI) m/z=527.2 [M+H]⁺. ¹H NMR (400 MHz, DMSO-d6) δ 9.50 (s, 1H), 9.32 (s, 1H), 7.96 (d, J=8.40 Hz, 1H), 7.72 (s, 1H), 7.58 (d, J=8.40 Hz, 1H), 7.44 (s, 1H), 7.29 (d, J=2.80 Hz, 2H), 6.83 (dd, J=9.20, 2.88 Hz, 1H), 4.83-4.92 (m, 1H), 3.33 (s, 13H), 3.08-3.17 (m, 4H), 2.39-2.47 (m, 5H), 2.21 (s, 3H), 1.28-1.33 (m, 2H), 1.23 (br s, 1H), 0.82-0.89 (m, 1H), 0.81-0.90 (m, 1H).

### Example 26: Synthesis of Compound 28

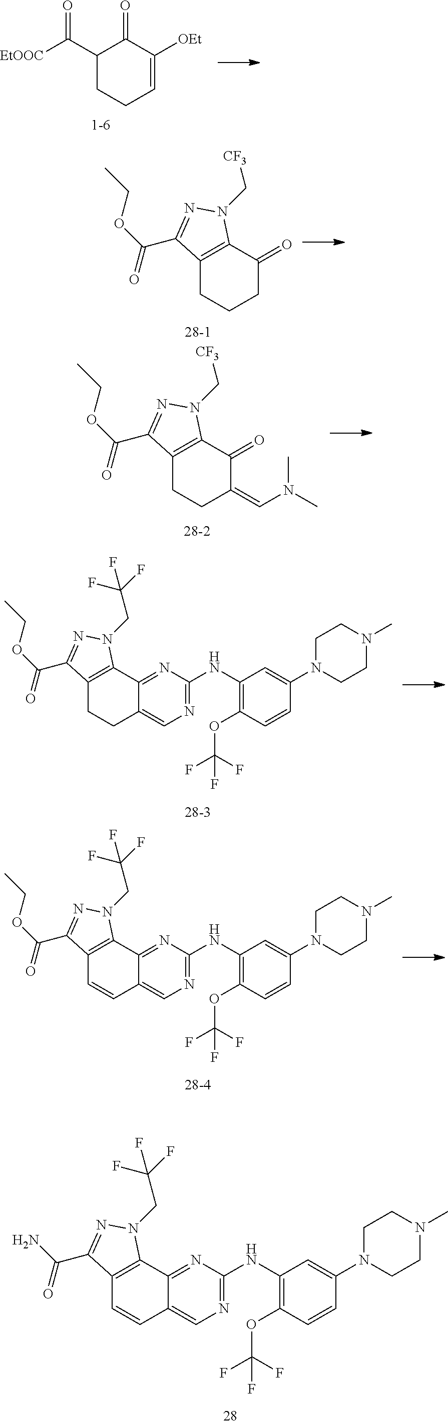

### 26.1 Synthesis of Intermediate 28-1

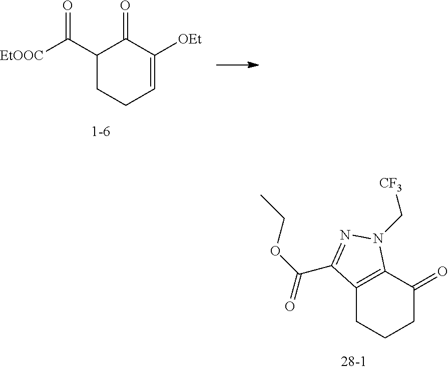 [SMILES: CCOC(=O)C(=O)C1CCC=C(OCC)C1=O ; CCOC(=O)c1nn(CC(F)(F)F)c2c1CCCC2=O]

Intermediate 1-6 (2.00 g, 8.32 mmol) was dissolved in acetic acid (20 mL), compound trifluoroethylhydrazine (1.14 g, 9.99 mmol) was added, and the mixture was reacted at room temperature for 15 h. Water (20 mL) was added to quench the reaction, saturated Na₂CO₃ (200 mL) was added, and dichloromethane (80 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-10%) to obtain intermediate 28-1 (1.18 g, yield 49.1%, yellow solid). MS (ESI) m/z=291.0 [M+H]⁺.

### 26.2 Synthesis of Intermediate 28-2

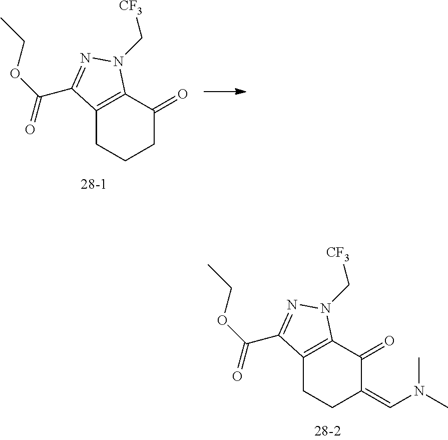 [SMILES: CCOC(=O)c1nn(CC(F)(F)F)c2c1CCCC2=O ; CCOC(=O)c1nn(CC(F)(F)F)c2c1CC/C(=C/N(C)C)C2=O]

Intermediate 28-1 (1.23 g, 4.24 mmol) was dissolved in DMF-DMA (30 mL) and the mixture was reacted at 110° C. for 12 h. The reaction liquid was concentrated under reduced pressure, and the residue was dissolved in methyl tertiary ether and the mixture was stirred for 3 h. The resulting product was filtered, the filter cake was collected, and dried to obtain intermediate 28-2 (1.4 g, crude product), which was directly used in the next step.

### 26.3 Synthesis of Intermediate 28-3

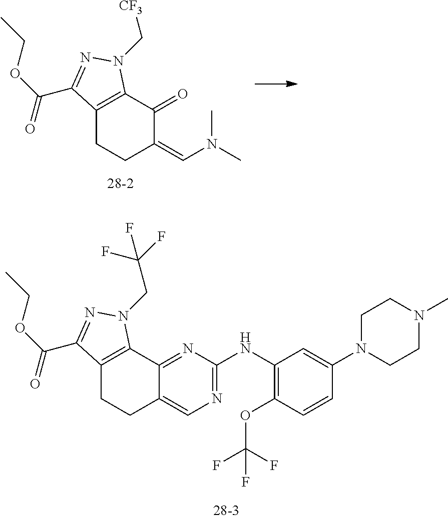 [SMILES: CCOC(=O)c1nn(CC(F)(F)F)c2c1CC/C(=C/N(C)C)C2=O ; CCOC(=O)c1nn(CC(F)(F)F)c2c1CCc1cnc(Nc3cc(N4CCN(C)CC4)ccc3OC(F)(F)F)nc1-2]

Intermediate 28-2 (1.23 g, 3.56 mmol) and intermediate 1-3 (1.36 g, 4.28 mmol) were dissolved in DMF (50 mL) and the mixture was reacted at 110° C. for 12 h. The reaction liquid was concentrated under reduced pressure, and the residue was subjected to silica gel column chromatography (ethyl acetate/petroleum ether=0%-5%) to obtain intermediate 28-3 (0.476 g, yield 22.3%, yellow solid). MS (ESI) m/z=600.6 [M+H]⁺.

### 26.4 Synthesis of Intermediate 28-4

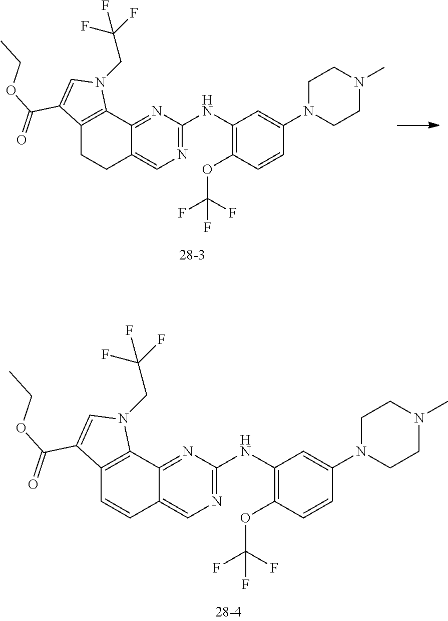

Intermediate 28-3 (0.61 g, 1.02 mmol) and DDQ (1.14 g, 5.04 mmol) were dissolved in dioxane (20 mL) and the mixture was reacted at 100° C. for 12 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (C18 150*40 mm*10 μm; mobile phase: [water (TFA)-acetonitrile]; B %: 50%-90%, 35 min). Intermediate 28-4 (0.078 g, yield 12.8%, brown solid) was obtained. MS (ESI) m/z=598.2 [M+H]⁺.

### 26.5 Synthesis of Compound 28

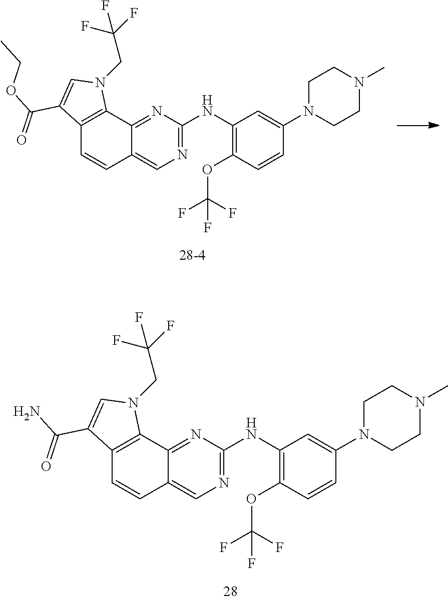 [SMILES: CCOC(=O)c1cn(CC(F)(F)F)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(N)=O)cn(CC(F)(F)F)c5c4n3)c2)CC1]

Intermediate 28-4 (50.0 mg, 83.7 μmol) was dissolved in NMP (6 mL), and NH₃/MeOH (50.0 mL) was added. The mixture was reacted for 48 h under sealed conditions at 80.0° C. The reaction liquid was concentrated under reduced pressure, and the residue was purified by preparative liquid chromatogram (C18 30*150 mm*7 μm; mobile phase: [water (TFA)-acetonitrile]; B %: 32%-72%, 36 min). Compound 28 (9.0 mg, yield 18.9%, yellow solid) was obtained. MS (ESI) m/z=569.1 [M+H]⁺. ¹H NMR (400 MHz, DMSO-d6) δ 9.66 (s, 1H), 9.36 (s, 1H), 8.00 (d, J=8.74 Hz, 1H), 7.55-7.84 (m, 3H), 7.25 (d, J=9.00 Hz, 1H), 6.90 (dd, J=9.14, 2.88 Hz, 1H), 5.80 (d, J=8.50 Hz, 2H), 3.12-3.20 (m, 4H), 2.40-2.46 (m, 4H), 2.21 (s, 3H).

### Example 27: Synthesis of Compound 29

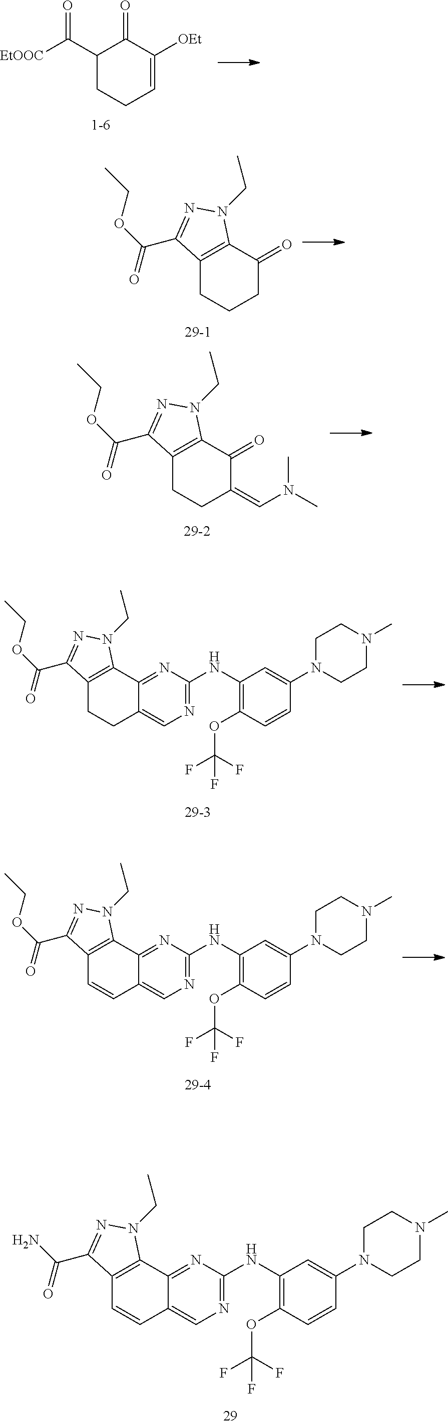

### 27.1 Synthesis of Intermediate 29-1

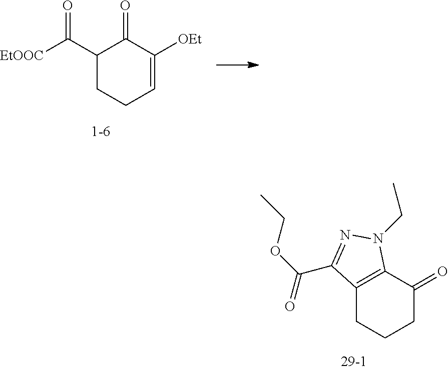 [SMILES: CCOC(=O)C(=O)C1CCC=C(OCC)C1=O ; CCOC(=O)c1nn(CC)c2c1CCCC2=O]

Intermediate 1-6 (1.60 g, 6.66 mmol) was dissolved in acetic acid (20 mL), compound ethylhydrazine (0.44 g, 7.33 mmol) was added, and the mixture was reacted at room temperature for 15 h. Water (20 mL) was added to quench the reaction, saturated Na₂CO₃ (200 mL) was added, and dichloromethane (80 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-10%) to obtain intermediate 29-1 (0.74 g, yield 47.1%, yellow solid). MS (ESI) m/z=237.1 [M+H]⁺.

### 27.2 Synthesis of Intermediate 29-2

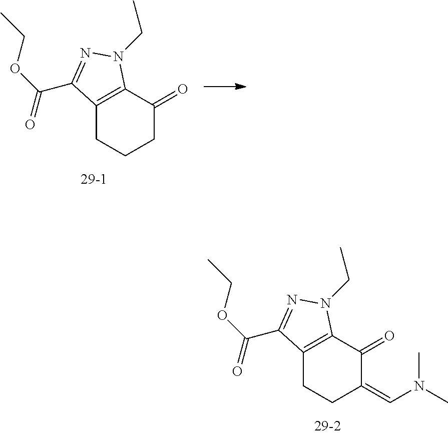 [SMILES: CCOC(=O)c1nn(CC)c2c1CCCC2=O ; CCOC(=O)c1nn(CC)c2c1CC/C(=C/N(C)C)C2=O]

Intermediate 29-1 (1.00 g, 3.81 mmol) was dissolved in DMF-DMA (30 mL) and the mixture was reacted at 110° C. for 12 h. The reaction liquid was concentrated under reduced pressure, and the residue was dissolved in methyl tertiary ether and the mixture was stirred for 3 h. The resulting product was filtered, the filter cake was collected, and dried to obtain intermediate 29-2 (1.0 g, crude product), which was directly used in the next step.

### 27.3 Synthesis of Intermediate 29-3

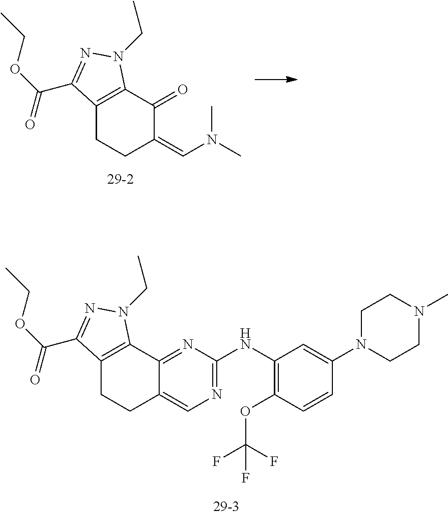 [SMILES: CCOC(=O)c1nn(CC)c2c1CC/C(=C/N(C)C)C2=O ; CCOC(=O)c1nn(CC)c2c1CCc1cnc(Nc3cc(N4CCN(C)CC4)ccc3OC(F)(F)F)nc1-2]

Intermediate 29-2 (1.00 g, 3.43 mmol) and intermediate 1-3 (1.09 g, 3.43 mmol) were dissolved in DMF (50 mL) and the mixture was reacted at 110° C. for 12 h. The reaction liquid was concentrated under reduced pressure, and the residue was subjected to silica gel column chromatography (methanol/dichloromethane=0%-10%) to obtain intermediate 29-3 (0.70 g, yield 37.4%, yellow solid). MS (ESI) m/z=546.2 [M+H]⁺.

### 27.4 Synthesis of Intermediate 29-4

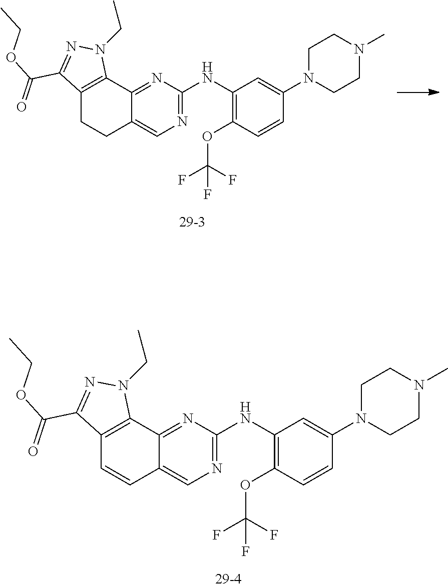

Intermediate 29-3 (0.70 g, 1.28 mmol) and DDQ (1.02 g, 4.49 mmol) were dissolved in dioxane (20 mL) and the mixture was reacted at 100° C. for 12 h. Saturated NaHCO₃ (20 mL) was added to quench the reaction, and ethyl acetate (20 mL×3) was added for extraction. The organic phases were combined, dried over anhydrous sodium sulfate, filtered, and the organic phase was concentrated under reduced pressure. The residue was purified by preparative liquid chromatogram (C18 150*40 mm*10 μm; mobile phase: [water (TFA)-acetonitrile]; B %: 45%-85%, 10 min). The intermediate 29-4 (0.3 g, yield 43%, yellow solid) was obtained. MS (ESI) m/z=544.3 [M+H]⁺.

### 27.5 Synthesis of Compound 29

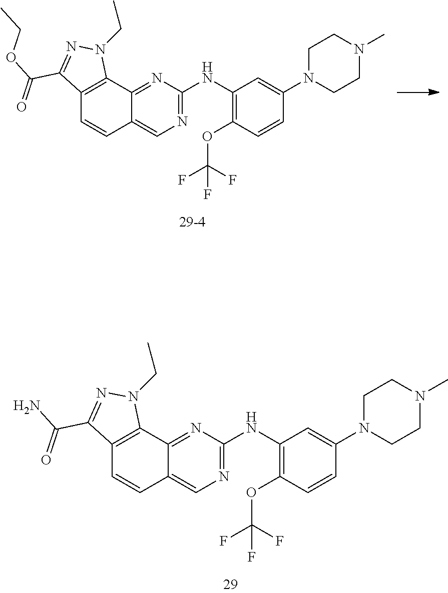 [SMILES: CCOC(=O)c1nn(CC)c2c1ccc1cnc(Nc3cc(N4CCN(C)CC4)ccc3OC(F)(F)F)nc12 ; CCn1nc(C(N)=O)c2ccc3cnc(Nc4cc(N5CCN(C)CC5)ccc4OC(F)(F)F)nc3c21]

Intermediate 29-4 (300 mg, 552 μmol) was dissolved in NMP (6 mL), and NH₃/MeOH (50.0 mL) was added. The mixture was reacted for 48 h under sealed conditions at 80.0° C. The reaction liquid was concentrated under reduced pressure, and the residue was purified by preparative liquid chromatogram (C18 30*150 mm*7 μm; mobile phase: [water (TFA)-acetonitrile]; B %: 32%-72%, 35 min). Compound 29 (72 mg, yield 25.3%, yellow solid) was obtained. MS (ESI) m/z=515.2 [M+H]⁺. ¹H NMR (400 MHz, DMSO-d6) δ 9.52 (br s, 1H), 9.31 (s, 1H), 7.98 (d, J=8.80 Hz, 1H), 7.76 (s, 1H), 7.57 (d, J=8.80 Hz, 1H), 7.45 (s, 1H), 7.27 (d, J=8.00 Hz, 1H), 7.21 (d, J=2.80 Hz, 1H), 6.88 (dd, J=9.20, 2.80 Hz, 1H), 4.82 (q, J=7.20 Hz, 2H), 3.08-3.25 (m, 4H), 2.42-2.43 (m, 4H), 2.21 (s, 3H), 1.23 (t, J=7.20 Hz, 3H).

### Example 28: Synthesis of Compound 30

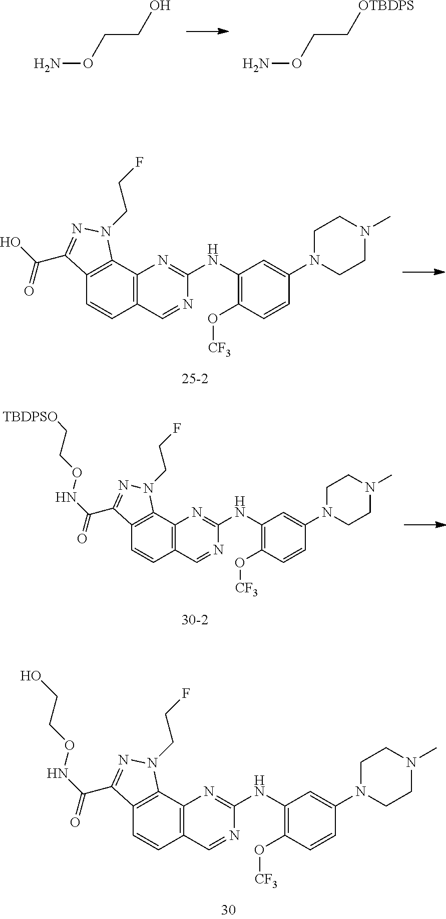

### 28.1 Synthesis of Intermediate 30-1

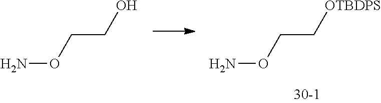

2-aminooxyethanol (1.00 g, 12.9 mmol) was dissolved in dichloromethane (20 mL), DMAP (0.158 g, 1.30 mmol), imidazole (1.32 g, 19.5 mmol) and TBDPSCl (4.28 g, 15.6 mmol) were added, and the mixture was reacted room temperature for 12 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-15%) to obtain intermediate 30-1 (1.59 g, yield 39%, oil). MS(ESI) m/z=316.1[M+H]⁺.

### 28.2 Synthesis of Intermediate 30-2

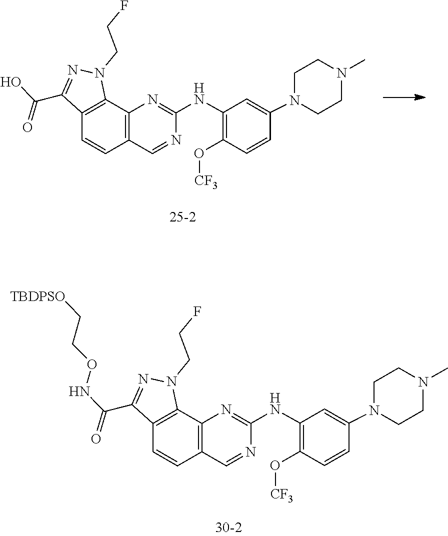

Compound 25-2 (50.0 mg, 93.7 μmol) was dissolved in DMF (3.00 mL), and HATU (39.2 mg, 103 μmol) was added. The obtained product was stirred at room temperature for 10 min, then DIEA (24.2 mg, 187 mol) and intermediate 30-1 (59.1 mg, 187 μmol) were added. The mixture was reacted at room temperature for 1 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The intermediate 30-2 (60.0 mg, crude product) was obtained and used directly in the next step. MS (ESI) m/z=831.5 [M+H]⁺.

### 28.3 Synthesis of Compound 30

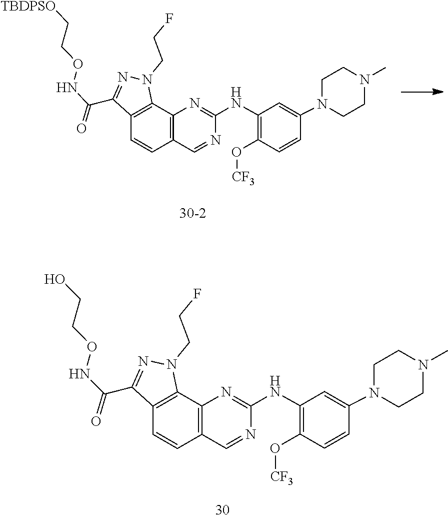

Intermediate 30-2 (50.0 mg, 60.2 μmol) was dissolved in DCM (5 mL), triethylamine trihydrofluoride (19.4 mg, 120 μmol) was added, and the mixture was reacted at room temperature for 5 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was purified by preparative liquid chromatogram (C18 30*150 mm*7 m; mobile phase: [water (TFA)-acetonitrile]; B %: 13%-60%, 14 min). Compound 30 (60 mg, yield 76.0%, yellow solid) was obtained. MS (ESI) m/z=593.0 [M+H]⁺. ¹H NMR (400 MHz, DMSO-d6) δ 9.59 (s, 1H) 9.34 (s, 1H) 7.93 (d, J=8.80 Hz, 1H) 7.63 (d, J=8.80 Hz, 1H) 7.26-7.35 (m, 1H) 7.19 (d, J=2.80 Hz, 1H) 6.89 (dd, J=9.20, 8.80 Hz, 1H) 5.06-5.22 (m, 2H) 4.69-4.81 (m, 2H) 4.61 (s, 1H) 3.95 (t, J=4.80 Hz, 2H) 3.62 (m, J=4.80 Hz, 2H) 3.14-3.22 (m, 4H) 2.40-2.45 (m, 4H) 2.21 (s, 3H).

### Example 29: Synthesis of Compound 31

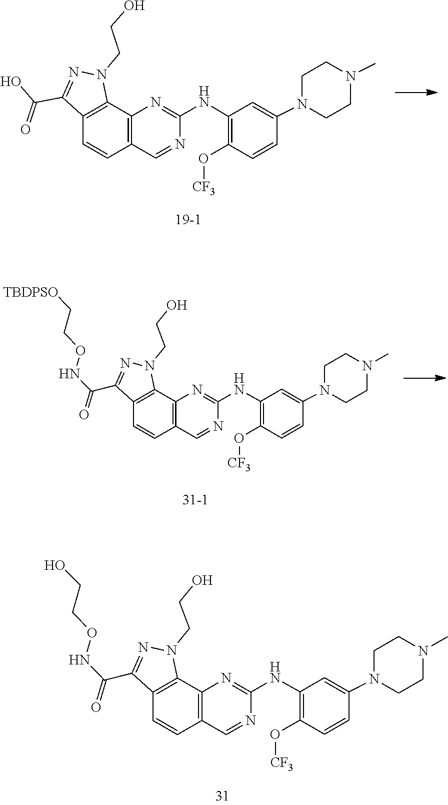

### 29.1 Synthesis of Intermediate 31-1

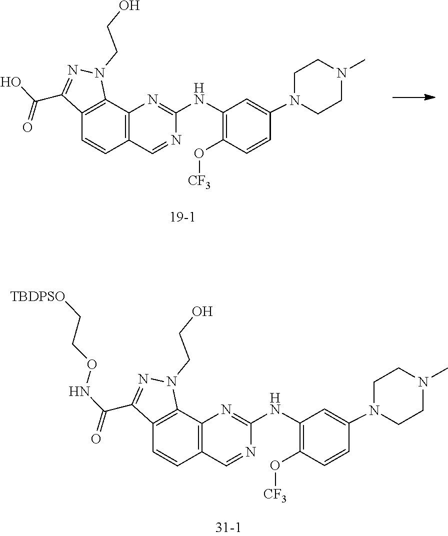

Compound 19-1 (50.0 mg, 94.1 μmol) was dissolved in DMF (3.00 mL), and HATU (39.4 mg, 103 μmol) was added. The obtained product was stirred at room temperature for 10 min, then DIEA (24.3 mg, 188 μmol) and intermediate 30-1 (59.4 mg, 188 μmol) were added. The mixture was reacted at room temperature for 12 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The intermediate 31-1 (60.0 mg, crude product) was obtained and used directly in the next step. MS (ESI) m/z=829.5 [M+H]⁺.

### 29.2 Synthesis of Compound 31

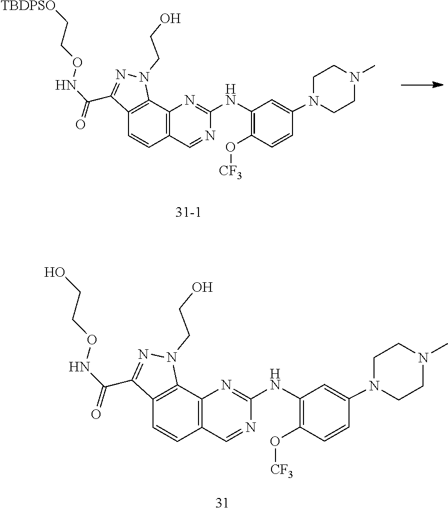

Intermediate 31-1 (50.0 mg, 60.3 μmol) was dissolved in DCM (5 mL), triethylamine trihydrofluoride (48.6 mg, 301 μmol) was added, and the mixture was reacted at room temperature for 5 h. Water (50 mL) was added to quench the reaction, and ethyl acetate (50 mL×3) was added for extraction. The organic phases were combined, dried over anhydrous sodium sulfate, filtered, and the organic phase was concentrated under reduced pressure. The residue was purified by preparative liquid chromatogram (C18 30*150 mm*7 μm; mobile phase: [water (TFA)-acetonitrile]; B %: 25%-85%, 14 min). Compound 31 (22.0 mg, yield 30.6%, yellow solid) was obtained. MS (ESI) m/z=591.3 [M+H]⁺. ¹H NMR (400 MHz, DMSO-d6) δ 9.50 (s, 1H) 9.32 (s, 1H) 7.90-8.00 (m, 1H) 7.61 (d, J=8.40 Hz, 1H) 7.19-7.31 (m, 2H) 6.88 (dd, J=9.07, 3.20 Hz, 1H) 4.88 (m, 2H) 4.76-4.84 (m, 1H) 4.57-4.63 (m, 1H) 3.88-3.96 (m, 2H) 3.55-3.72 (m, 5H) 3.15-3.21 (m, 4H) 2.42-2.46 (m, 4H) 2.18-2.22 (m, 1H) 2.22 (s, 3H).

### Example 30: Synthesis of Compound 32

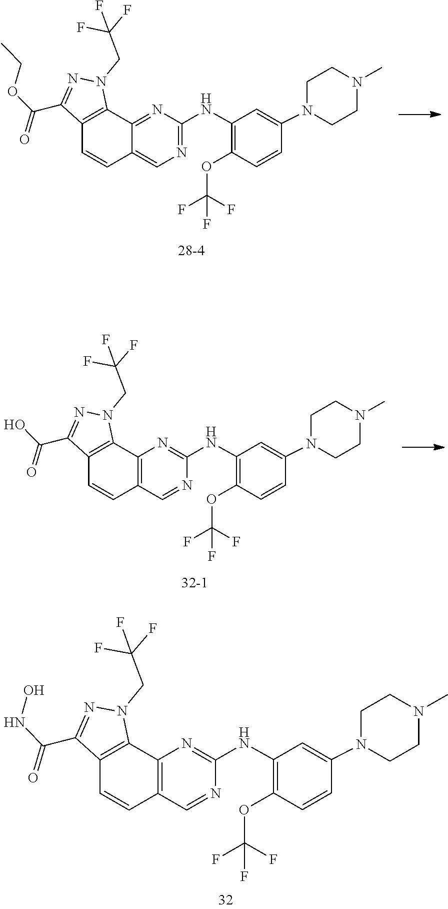 [SMILES: CCOC(=O)c1nn(CC(F)(F)F)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)nn(CC(F)(F)F)c5c4n3)c2)CC1 ; CN1CCN(c2ccc(OC(F)(F)F)c(Nc3ncc4ccc5c(C(=O)NO)nn(CC(F)(F)F)c5c4n3)c2)CC1]

### 30.1 Synthesis of Intermediate 32-1

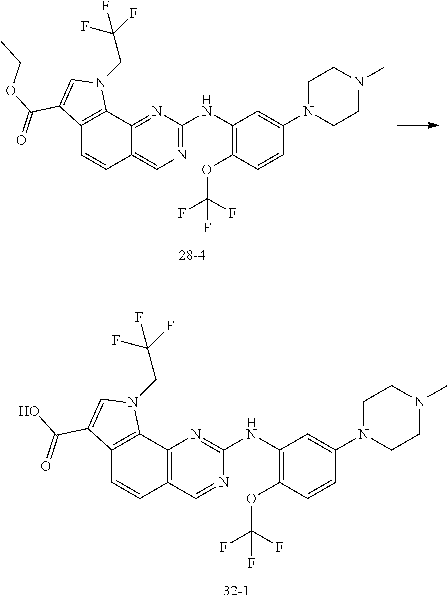 [SMILES: CCOC(=O)c1cn(CC(F)(F)F)c2c1ccc1cnc(Nc3cc(N4CCN(C)CC4)ccc3OC(F)(F)F)nc12 ; CN1CCN(c2ccc(OC(F)(F)F)c(Nc3ncc4ccc5c(C(=O)O)cn(CC(F)(F)F)c5c4n3)c2)CC1]

Intermediate 28-4 (50.0 mg, 83.7 μmol) was dissolved in methanol (5 mL), and lithium hydroxide aqueous solution (1.00 M, 418 μL) was added. The mixture was reacted at room temperature for 2 h. The reaction liquid was adjusted to acidic pH with hydrochloric acid, and concentrated under reduced pressure, and the residue was purified by preparative liquid chromatogram (C18 30*150 mm*7 μm; mobile phase: [water (TFA)-acetonitrile]; B %: 20%-50%, 10 min). The intermediate 32-1 (40.0 mg, yield 83.8%, yellow solid) was obtained. MS (ESI) m/z=570.1 [M+H]⁺.

### 30.2 Synthesis of Compound 32

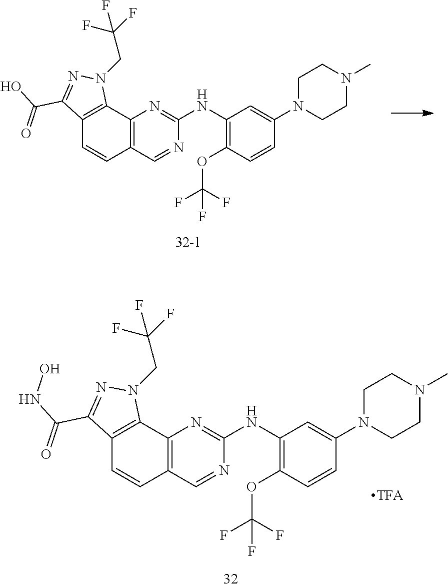 [SMILES: CN1CCN(c2ccc(OC(F)(F)F)c(Nc3ncc4ccc5c(C(=O)O)nn(CC(F)(F)F)c5c4n3)c2)CC1 ; CN1CCN(c2ccc(OC(F)(F)F)c(Nc3ncc4ccc5c(C(=O)NO)nn(CC(F)(F)F)c5c4n3)c2)CC1]

Intermediate 32-1 (40.0 mg, 70.2 μmol), HATU (29.4 mg, 77.1 μmol), DIEA (18.2 mg, 140 μmol), and NH₂OH (9.76 mg, 140 μmol) were dissolved in DMF (2 mL) and the mixture was reacted at room temperature for 2 h. The reaction liquid was concentrated under reduced pressure, and the residue was purified by preparative liquid chromatogram (C18 30*150 mm*7 μm; mobile phase: [water (TFA)-acetonitrile]; B %: 10%-50%, 12 min). Compound 32 (5.0 mg, yield 12.2%, brown solid) was obtained. MS (ESI) m/z=585.3 [M+H]⁺.

### Example 31: Synthesis of Compound 33

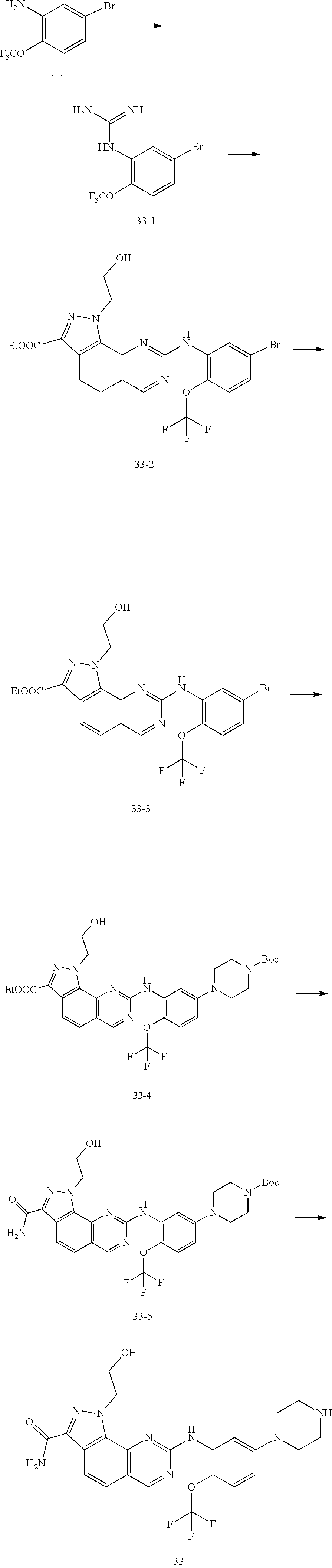

### 31.1 Synthesis of Intermediate 33-1

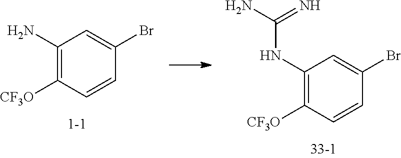

Compound 1-1 (13.0 g, 50.8 mmol) was dissolved in water (130 mL), and concentrated hydrochloric acid (12 mL) and cyanamide (10.6 g, 254 mmol) were added. The mixture was reacted at 60° C. for 16 h. The mixture was adjusted to pH=14 with 40% sodium hydroxide solution, and extracted with ethyl acetate (150 mL×3). The organic phases were combined, dried over anhydrous sodium sulfate, filtered, and the organic phase was concentrated under reduced pressure. The intermediate 33-1 (10.9 g, yield 72.2%, brown solid) was obtained. MS (ESI) m/z=297.9 [M+H]⁺.

### 31.2 Synthesis of Intermediate 33-2

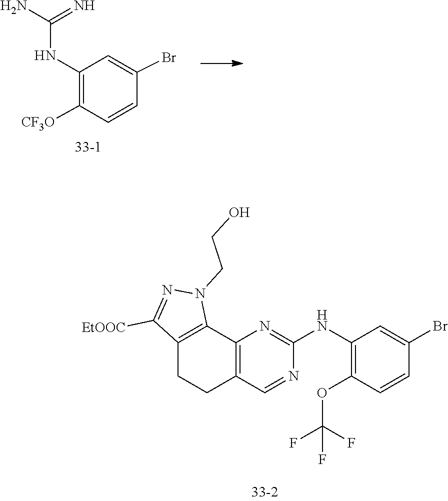

Intermediate 33-1 (5.00 g, 16.8 mmol) was dissolved in DMF (50 mL), intermediate 1-8 (5.16 g, 16.8 mmol) was added, and the mixture was reacted at 110° C. for 16 h. Water (200 mL) was added to quench the reaction, and ethyl acetate (100 mL×3) was added for extraction. The organic phases were combined, dried over anhydrous sodium sulfate, filtered, and the organic phase was concentrated under reduced pressure. The residue was subjected to silica gel column chromatography (ethyl acetate/petroleum ether=0%-100%) to obtain intermediate 33-2 (2.96 g, yield 32.5%, yellow solid). MS (ESI) m/z=542.3 [M+H]⁺.

### 31.3 Synthesis of Intermediate 33-3

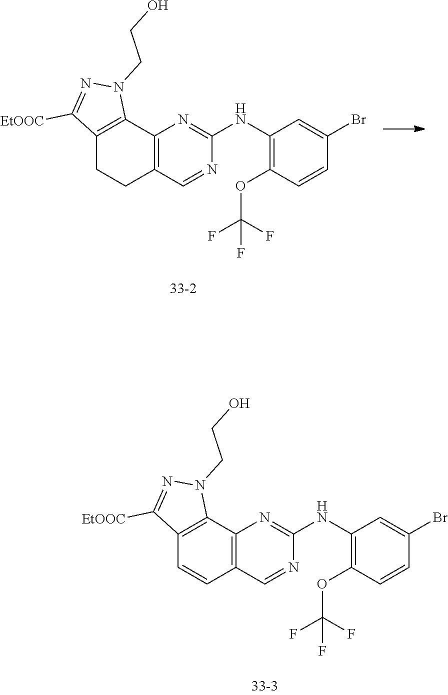 [SMILES: CCOC(=O)c1nn(CCO)c2c1CCc1cnc(Nc3cc(Br)ccc3OC(F)(F)F)nc1-2 ; CCOC(=O)c1nn(CCO)c2c1ccc1cnc(Nc3cc(Br)ccc3OC(F)(F)F)nc12]

Intermediate 33-2 (2.55 g, 4.70 mmol) and DDQ (2.35 g, 10.34 mmol) were dissolved in dioxane (60 mL) and the mixture was reacted at 100° C. for 9 h. The resulting product was filtered, the filtrate was collected, and concentrated under reduced pressure to obtain intermediate 33-3 (2 g, crude product), which was directly used in the next step. MS (ESI) m/z=540.0 [M+H]⁺.

### 31.4 Synthesis of Intermediate 33-4

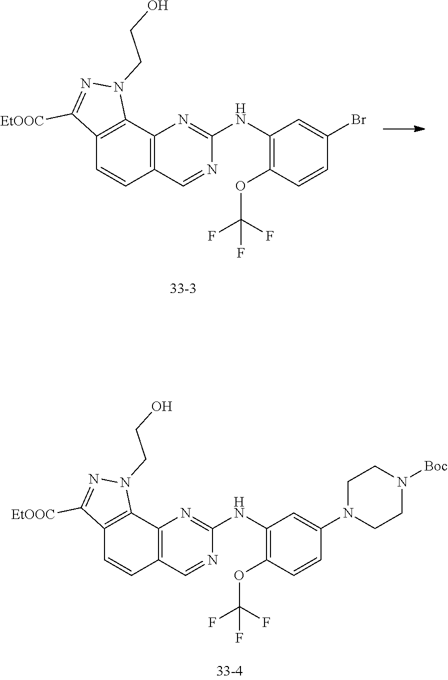 [SMILES: CCOC(=O)c1nn(CCO)c2c1ccc1cnc(Nc3cc(Br)ccc3OC(F)(F)F)nc12 ; CCOC(=O)c1nn(CCO)c2c1ccc1cnc(Nc3cc(N4CCN(C(=O)OC(C)(C)C)CC4)ccc3OC(F)(F)F)nc12]

Intermediate 33-3 (1.20 g, 1.49 mmol, purity 67%) and acetate salt of tert-butyl piperazine-1-carboxylate (1.39 g, 7.44 mmol) were dissolved in dioxane (6 mL), XPhos (354 mg, 744 μmol), KOtBu (334 mg, 2.98 mmol) and Pd₂(dba)₃ (341 mg, 372 μmol) were added. The mixture was reacted at 75° C. for 20 h. Water (100 mL) was added to quench the reaction, and ethyl acetate (100 mL×3) was added for extraction. The organic phases were combined, dried over anhydrous sodium sulfate, filtered, and the organic phase was concentrated under reduced pressure. The residue was purified by preparative liquid chromatogram (C18 30*150 mm*7 μm; mobile phase: [water (NH₃H₂O+NH₄HCO₃)-acetonitrile]; B %: 30%-70%, 20 min). The intermediate 33-4 (0.12 g, yield 12.5%, brown solid) was obtained. MS (ESI) m/z=646.2 [M+H]⁺.

### 31.5 Synthesis of Intermediate 33-5

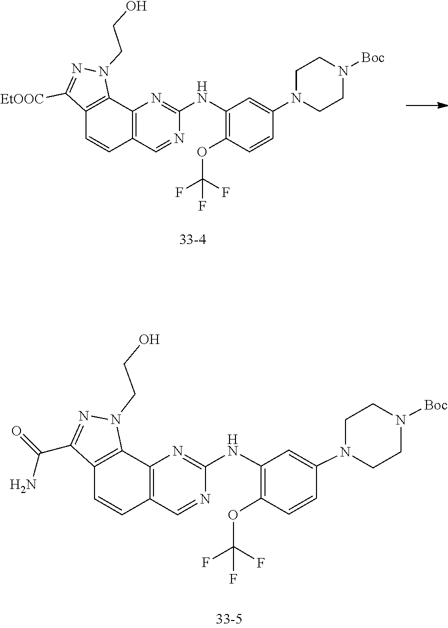 [SMILES: CCOC(=O)c1nn(CCO)c2c1ccc1cnc(Nc3cc(N4CCN(C(=O)OC(C)(C)C)CC4)ccc3OC(F)(F)F)nc12 ; CC(C)(C)OC(=O)N1CCN(c2ccc(OC(F)(F)F)c(Nc3ncc4ccc5c(C(N)=O)nn(CCO)c5c4n3)c2)CC1]

Intermediate 33-4 (90.0 mg, 139 μmol) was dissolved in NH₃/MeOH (10 mL, 7 M) and the mixture was reacted at 80° C. for 48 h. After cooling to room temperature, the reaction liquid was concentrated under reduced pressure to obtain intermediate 33-5 (100 mg, crude product), which was directly used in the next step. MS (ESI) m/z=617.4 [M+H]⁺. 31.6 Synthesis of compound 33

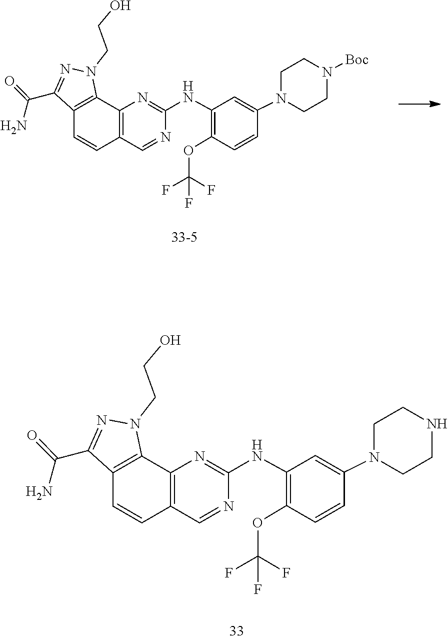 [SMILES: CC(C)(C)OC(=O)N1CCN(c2ccc(OC(F)(F)F)c(Nc3ncc4ccc5c(C(N)=O)nn(CCO)c5c4n3)c2)CC1 ; NC(=O)c1nn(CCO)c2c1ccc1cnc(Nc3cc(N4CCNCC4)ccc3OC(F)(F)F)nc12]

Intermediate 33-5 (90.0 mg, 124 μmol, 85% purity) was dissolved in dichloromethane (5 mL), TFA (212 mg, 1.86 mmol) was added, and the mixture was reacted at room temperature for 4 h. The reaction liquid was concentrated under reduced pressure, and the residue was purified by preparative liquid chromatogram (C18 30*150 mm*7 μm; mobile phase: [water (TFA)-acetonitrile]; B %: 4%-44%, 36 min). The prepared freeze-dried product was dissolved in water and acetonitrile (volume ratio 1:1), a solid was precipitated, filtered by suction, and dried. Compound 33 (30.3 mg, yield 57.2%, yellow solid) was obtained. MS (ESI) m/z=517.4 [M+H]⁺. ¹H NMR (400 MHz, DMSO-d6) δ 9.44 (s, 1H), 9.31 (s, 1H), 8.00 (d, J=8.8 Hz, 1H), 7.74 (br s, 1H), 7.58 (d, J=8.8 Hz, 1H), 7.44 (br s, 1H), 7.19-7.30 (m, 2H), 6.85 (dd, J=9.2, 2.8 Hz, 1H), 4.89 (t, J=4.8 Hz, 2H), 4.59 (br s, 1H), 3.64-3.73 (m, 2H), 3.00-3.15 (m, 4H), 2.76-2.90 (m, 4H).

### Test Example 1. Inhibitory Activity on PLK1 Kinase

1. Test materials: PLK1 kinase (Carnabio #05-157)

Detection solution PerkinElmer #TRF0218-M

Detection instrument PerkinElemer EnVision

### 2. Test Method:

a. Using the Echo machine, the compounds to be tested were diluted by 1:3 on a 384-well plate, and the highest concentration was 1 μM, with a total of 10 concentration points, and each concentration was tested in duplicate wells.

b. 5 μL/well of PLK1 kinase and peptide substrate mixture were added to the assay plate (containing controls). Each assay plate was set with GSK461364 as a positive control and DMSO solvent group as a negative control.

c. The assay plate was centrifuged at 1000 rpm for approximately 15 seconds and incubated at 23° C. for 15 min.

d. The reaction was initiated by adding 5 μL/well of ATP solution;

e. The assay plate was centrifuged at 1000 rpm for about 15 seconds, a film was used to seal the assay plate which was incubated at 23° C. for a period of time;

f. A detection solution was added to all wells of the assay plate;

g. The assay plate was centrifuged at 1000 rpm for approximately 15 seconds, a film was used to seal the assay plate which was incubated at 23° C. for 60 min.

h. The assay plate was read on EnVision. The test results were analyzed using XLFIT5 software.

3. Test results: The IC₅₀ and Iₘₐₓ (%) for the inhibitory activity of the compound of the present invention on PLK1 kinase are shown in Table 1. Iₘₐₓ is the maximum effect (S/B 8.52, Z factor 0.94) caused by the compound at the test concentration.

TABLE 1
Inhibitory test results on PLK1 kinase

| Compound No. | IC₅₀ (nM) | Iₘₐₓ (%) |
|---|---|---|
| GSK461364¹ | 0.84 | 101.08 |
| Onvansertib² | 1.08 | 100.02 |
| 1 | 1.81 | 99.35 |
| 2 | 0.99 | 99.81 |
| 3 | 0.714 | 99.57 |
| 4 | 1.64 | 99.51 |
| 5 | 73.29 | 76.81 |
| 7 | 0.79 | 100.68 |
| 8 | 14.7 | 95.96 |
| 9 | 327.07 | 72.68 |
| 10 | 1.16 | 99.92 |
| 11 | 1.80 | 99.65 |
| 12 | 2.76 | 99.53 |
| 13 | 2.49 | 99.41 |
| 16 | 4.77 | 98.76 |
| 17 | 0.814 | 99.97 |
| 18 | 0.661 | 100.19 |
| 19 | 1.06 | 99.97 |
| 20 | 1.28 | 99.29 |
| 21 | 0.756 | 99.15 |
| 22 | 1.51 | 99.99 |
| 23 | 3.66 | 98.18 |
| 24 | 1.21 | 99.48 |
| 25 | 1.12 | 100.23 |
| 27 | 0.459 | 100.24 |
| 28 | 0.670 | 100.08 |
| 29 | 0.805 | 100.08 |
| 30 | 0.776 | 99.97 |
| 31 | 1.55 | 99.54 |
| 32 | 0.463 | 99.87 |

¹The compound prepared and disclosed in Example 3 of CN 101300251 B;
²The compound prepared and disclosed in Example 38 of CN 101563351 B.

Conclusion: The IC₅₀ of most compounds of the present invention in inhibiting PLK1 kinase is at the level of 1-2 nM, which is equivalent to the control drug onvansertib. The kinase activity of compounds 3, 7, 17, 18, 21, 27-30, and 32 is higher than that of the control drug.

### Test Example 2. Inhibitory Activity on HCT116 Cell Proliferation

### 1. Test Materials:

Cell lines: HCT116 cells

Cell culture medium: RPMI1640+10% FBS (GBICO)

Cell culture plate: 96-well plate (Shanghai Jingan Biotechnology Co., Ltd.)

Detection kit: ATPlite 1step Luminescence (PerkinElmer)

Detection instrument: BioTek multifunctional microplate reader

2. Test method: HCT116 cells in the logarithmic growth phase were seeded into a white-wall clear-bottom 96-well plate at a density of 720 cells per well, and cultured overnight in a 37° C., 5% CO₂ incubator. The next day, a compound to be tested was added to the cells, wherein the test compound had a concentration of 10 M and was subjected to 3-fold gradient dilution to obtain 9 concentrations, and double replicate wells were set up for each concentration. The blank culture medium was used as a negative control, and the same concentration of Onvansertib was used as a positive control. After adding the drug, culturing was continued for 72 h in a 37° C., 5% CO₂ incubator. ATPlite 1step Luminescence reagent with the same volume as the cell fluid was added to the cells, incubated in the dark for 3 min at room temperature, and oscillated for 2 min with a micro-oscillator at 500 r, and the luminescent intensity was detected by a microplate reader to calculate the cell inhibition rate.

$$Cell inhibition rate ( \% ) = [ 100 - ( {Lum}_{sample to be tested} - {Lum}_{culture solution} ) / ( {Lum}_{negative control} - {Lum}_{culture solution} ) \times 100 ] \% .$$

GraphPad Prism 7.0 was used to process the data, the cell inhibition rate curve was obtained and IC₅₀ was calculated.

### 3. Test Results:

The half inhibitory concentration IC₅₀ of the compound of the present invention on HCT116 cell proliferation is shown in Table 2.

TABLE 2
IC₅₀ of the compound of the present invention on HCT116 cells

| Compound No. | IC₅₀ (μM) |
|---|---|
| Onvansertib¹ | 0.042 |
| 1 | 0.051 |
| 2 | 0.027 |
| 3 | 0.093 |
| 4 | 0.332 |
| 5 | 0.924 |
| 6 | 2.724 |
| 8 | 3.401 |
| 9 | >10* |
| 10 | 0.049 |
| 11 | 0.205 |
| 12 | 0.861 |
| 13 | 0.831 |
| 16 | 0.502 |
| 18 | 0.005 |
| 19 | 0.096 |
| 20 | 0.143 |
| 23 | 0.214 |
| 24 | 0.032 |
| 29 | 0.077 |

¹The compound prepared and disclosed in Example 38 of CN 101563351 B.
*indicates that the inhibitory effect of the compound is less than 50% at 10 μM.

Conclusion: The half inhibitory concentration of compound 1 of the present invention on HCT116 cells is 51 nM, and the half inhibitory concentration of compound 10 on HCT116 cells is 49 nM, which are equivalent to the control Onvansertib. The half inhibitory concentration of compound 2 on HCT116 cells is 27 nM, the half inhibitory concentration of compound 24 on HCT116 cells is 32 nM, and the half inhibitory concentration of compound 18 on HCT116 cells is 5 nM. Compared to the control drug Onvansertib, the activity is enhanced.

### Test Example 3. Evaluation of Permeability of Test Substance and Transporter Substrate

### 1. Test Materials:

In this experiment, Caco-2 cells were seeded in Transwell-96 well plates at a seeding density of 1×10⁵ cells/cm². The cells were cultured in a carbon dioxide incubator for 21-28 days before being used for transport experiments. During this period, the culture medium was changed every four to five days.

### 2. Test Method:

This project uses Hank's balanced salt buffer (pH 7.40±0.05) containing 10 mM HEPES as the transport buffer. The culture medium was removed from the culture plate and the cells were washed twice with preheated transport buffer (approximately 75 μL and 40 mL of transport buffer for each apical well and basolateral receiver plate respectively). The donor solution and receiver solution were added to the corresponding cell plate wells (75 and 250 μL for each apical and basolateral well respectively), and the bidirectional transport experiment was started (apical to basolateral (A-B) and basolateral to apical (B-A)). The test concentration of the test substance was 5.00 M, and two replicate wells were made for each dosing concentration. The test concentration of Digoxin was 10.0 M, with dosing bidirectionally; the test concentrations of nadolol and metoprolol were both 2.00 M, and they were dosed in one direction (A-B direction), and two replicate wells were made for each of the three control compounds. After adding the sample, the cell plate was incubated at 37±1° C., 5% CO₂ and saturated humidity for 120 min.

The initial donor solution is the TO sample. After adding the sample, it was mixed with the transport buffer and stop solution in a certain proportion. After incubation for 120 min, the final samples were collected from the donor side and receiver side, and were also mixed with transport buffer and stop solution in a certain proportion.

All samples were vortexed, and then centrifuged at 3220×g for 20 min at 20° C. A proper volume of supernatant was transferred to a sample assay plate, and the sample was stored at 2-8° C. if it was not analyzed immediately after the plate was sealed, and LC MS/MS method was used for analysis.

The Lucifer Yellow Rejection Assay was used to test the integrity of the Caco-2 cell layer.

### 3. Data Analysis:

The following formulas were used to calculate the apparent permeability coefficient (Pₐₚₚ, cm/s), efflux ratio (ER), and solution recovery (%) (% Solution Recovery).

$${P}_{app} = \frac{{V}_{R}}{Area \times Time} \times \frac{{[ drug ]}_{recevier}}{{[ drug ]}_{initial , donor}} = \frac{{V}_{R}}{Area \times Time} \times \frac{{C}_{R}}{{C}_{0}}$$

$$ER = \frac{{P}_{app} ( B - A )}{{P}_{app} ( A - B )}$$

$$\% SolutionRecovery = \frac{{C}_{R} \times {V}_{R} + {C}_{D} \times {V}_{D}}{{C}_{0} \times {V}_{D}} \times 100$$

V_{R} is the volume of the receiver side solution (side A is 0.075 mL, side B is 0.25 mL); Area is the relative surface area of the cell monolayer (0.0804 cm²); Time is the incubation time (7200 s); C₀ is the initial concentration (nM) of the test substance at the donor side or the peak area ratio of the control compound; V_{D} is the volume of the donor side (side A is 0.075 mL, side B is 0.25 mL); C_{D} and C_{R} are the final concentration (nM) of the test substance at the donor side and the receiver side respectively or the peak area ratio of the control compound;
Lucifer yellow transmittance rate (% Lucifer Yellow) is calculated using the following formula:

$$\% Lucifer Yellow = \frac{{V}_{Basolateral} \times {RFU}_{Basolateral}}{{V}_{Apical} \times {RFU}_{Apical} + {V}_{Basolateral} \times {RFU}_{Basolateral}} \times 100$$

RFUApical and RFUBasolateral are the relative fluorescence intensity of lucifer yellow at the apical and basolateral sides respectively. VApical and VBasolateral are the sample loading volumes at the apical and basolateral sides respectively (0.075 and 0.25 mL respectively).

### 4. Classification Criteria:

| Parameter | Classification | Standard |
|---|---|---|
| Permeability | Low | Pₐₚₚ (A − B) ≤ 0.500 |
| | | (×10⁻⁶ cm/s) |
| | Medium | 0.500 < Pₐₚₚ (A − B) < 2.50 |
| | | (×10⁻⁶ cm/s) |
| | High | Pₐₚₚ (A − B) ≥ 2.50 |
| | | (×10⁻⁶ cm/s) |
| Efflux transporter | Likely | ERₐ ≥ 2.00 |
| substrate | Very unlikely | ERₐ < 2.00 |
| | or not | |

5. Test results: The permeability results of the compounds of the present invention in Caco-2 cells are shown in Table 5.

TABLE 5
Permeability results in Caco-2 cells

| | Average Pₐₚₚ | | | Classification | |
|---|---|---|---|---|---|
| Compound | (10⁻⁶ cm/s) | | Efflux | | Efflux transporter |
| No. | A − B | B − A | ratio | Permeability | substrate |
| Onvansertib | 0.299 | 43.6 | 146 | Low | Likely |
| 1 | 0.965 | 20.8 | 21.5 | Medium | Likely |
| 2 | <0.480 | 26.1 | >54.4 | Low | Likely |
| 3 | 0.644 | 0.851 | 1.32 | Medium | Very unlikely or not |
| 4 | 1.16 | 25.5 | 22.1 | Medium | Likely |
| 7 | 0.275 | 0.542 | 1.97 | Low | Very unlikely or not |
| 8 | <0.480 | 14.5 | >30.3 | Low | Likely |
| 9 | <0.254 | 6.43 | >25.4 | Low | Likely |
| 10 | <0.480 | 24.8 | >51.7 | Low | Likely |
| 11 | 1.13 | 25.0 | 22.1 | Medium | Likely |
| 12 | <0.264 | 15.0 | >56.7 | Low | Likely |
| 13 | <0.480 | 13.7 | >28.6 | Low | Likely |
| 16 | 0.917 | 6.66 | 7.27 | Medium | Likely |
| 18 | 2.03 | 3.36 | 1.65 | Medium | Very unlikely or not |
| 20 | 1.30 | 13.4 | 10.4 | Medium | Likely |
| 23 | <0.280 | 23.1 | >82.6 | Low | Likely |
| 24 | 0.106 | 29.5 | 279 | Low | Likely |

Conclusion: The compounds of the present invention are low or moderately permeable in Caco-2 cells.

### Test Example 4. Evaluation of Metabolic Stability of Liver Microsomes

### 1. Reagents:

| Name | Content | Batch No. | Source |
|---|---|---|---|
| Tetrasodium salt | 98% | 50650124 | Rocheroche |
| of nicotinamide | | | Diagnostics GmbH |
| adenine dinucleotide | | | |
| phosphate | | | |
| (NADPH) | | | |
| Phosphate buffered | / | SLBX1022 | SIGMA |
| saline | | | |
| Magnesium chloride | ≥99.0% | BCBW0417 | SIGMA-ALDRICH |
| hexahydrate | | | |
| (MgCl₂•6H₂O) | | | |
| Formic acid (FA) | ≥96.0% | SHBJ0951 | SIGMA-ALDRICH |
| Acetonitrile | / | JA104130 | Merck KGaA |
| Methanol | / | I10099407026 | Merck KGaA |
| Ultra-pure water | / | / | Prepared from MilliQ |
| | | | water machine |

### 2. Equipment

| Name | Model | Manufacturer |
|---|---|---|
| Chromatographic column | 2.1 × 150 mm, | Agilent |
| Agilent XDB C18 | 3.5 micro | |
| Quadrupole high-resolution | Q Exactive | Thermoscientific |
| mass spectrometer | | |
| Ultra high performance | HCLass | Waters |
| liquid chromatograph | | |
| Electric heating constant | DKZ | Shanghai Yiheng |
| temperature oscillating | | Technology Co., Ltd. |
| water tank | | |
| High speed desktop | XIR | Thermo |
| centrifuge | | |
| Centrifuge | Legend Micro | Thermo |
| | 21R | |
| Ultrapure water instrument | Advantage A10 | Millipore |
| Electronic balance | XS105 | METTLER TOLEDO |

### 3. Test Method

a. Preparation of phosphate buffer: The phosphate buffer powder was dissolved in 100 mL of pure water to prepare a phosphate buffer solution with a pH of 7.4 and a concentration of 100 mM, which was stored in a 4° C. refrigerator for later use.

b. All samples as well as the reference testosterone were dissolved in DMSO to obtain a 10 mM stock solution. The 10 mM stock solution was diluted to 100 μM with acetonitrile to obtain a working solution for later use.

c. Preparation of liver microsome working solution: Each of the mouse, rat and human liver microsome stock solutions (at concentration of 20 mg/mL) was taken and diluted to 0.56 mg/mL with 100 mM phosphate buffer solution to obtain the liver microsome working solution.

d. Preparation of solution: An appropriate amount of MgCl2 was weighed and used to prepare a MgCl2 solution at a concentration of 60 mM using 100 mM phosphate buffer solution. An appropriate amount of NADPH was weighed and used to prepare a NADPH solution at a concentration of 20 mM using 100 mM phosphate buffer solution. An equal volume of 60 mM MgCl₂ solution was added to prepare an NADPH working solution containing 10 mM NADPH and 30 mM MgCl₂. An acetonitrile solution containing 20 ng/mL tolbutamide was prepared as the stop solution.

e. Liver microsome incubation: 2 μL of each compound and the positive drug testosterone working solution were added to 178 μL of liver microsome working solution (0.56 mg/mL), mixed gently, and pre-incubated in a vibrating constant temperature water bath pot at 37° C. for 10 min. After adding 20 μL of NADPH working solution, the mixture was placed in a vibrating constant temperature water bath pot at 37° C. and timed incubation was started for 5, 10, 20, 30 and 60 min.

f. Stopping reaction: After the corresponding time of incubation, 400 μL of stop solution was added to stop the reaction, and then the sample was immediately shaken for 1 min. The sample was centrifuged at 13500 rpm for 10 min at 10° C. After centrifugation, the supernatant was removed, diluted with water according to the needs of each compound, and analyzed by LC-MS. For the 0-minute sample, the stop solution was first added and mixed evenly with the liver microsomes, and then the NADPH working solution was added. For the negative control sample, 20 μL of 30 mM MgCl2 solution was added instead of the NADPH working solution.

### 4. Data Analysis:

T_{1/2} and CL_{int(mic)} were calculated by the following formula.

$${C}_{t} = {C}_{0} * {e}^{- {k}_{{e}^{\cdot t}}}$$

$$When {C}_{t} = 1 / 2 {C}_{0} , {T}_{1 / 2} = ln 2 / {k}_{e} = 0.693 / {k}_{e}$$

CL_{int(mic)}=0.693/T_{1/2}/liver microsomal protein concentration during incubation (mg/mL)
CLᵢₙₜ₍ᵥₑᵣ₎=CL_{int(mic)}*liver microsomal protein (mg)/liver weight (g)*liver weight to body weight ratio

Liver microsomal protein (mg)/liver weight (g): the value is 45 in both animal and human species.

Liver weight to body weight ratio: the parameters for mice, rats and humans are 88, 40 and 20 g/kg respectively.

5. Test results: The metabolic stability results of the compounds of the present invention in mouse, rat and human liver microsomes are shown in Table 6.

TABLE 6
Metabolic stability results in mouse, rat and human liver microsomes

| | | 60 min remaining amount | T_{1/2} | CL_{int(mic)} | CLᵢₙₜ₍ₗᵢᵥₑᵣ₎ | |
|---|---|---|---|---|---|---|
| Compound | Species | % | min | μL/min/mg protein | mL/min/kg | Clearance rate |
| Onvansertib | Mouse | 21.5 | 27.2 | 51.0 | 202 | Medium |
| | Rat | 15.7 | 22.7 | 61.0 | 110 | Medium |
| | Human | 64.8 | 81.5 | 17.0 | 15.3 | Medium |
| 1 | Mouse | 19.2 | 26.7 | 52.0 | 206 | Medium |
| | Rat | 3.06 | 12.2 | 113 | 204 | High |
| | Human | 26.0 | 30.8 | 45.0 | 40.5 | Medium |
| 2 | Mouse | 26.7 | 31.4 | 44.2 | 175 | Medium |
| | Rat | 17.0 | 24.3 | 57.0 | 103 | Medium |
| | Human | 49.9 | 55.0 | 25.2 | 22.7 | Medium |
| 3 | Mouse | 4.32 | 13.4 | 103 | 409 | High |
| | Rat | 2.74 | 4.94 | 281 | 505 | High |
| | Human | 7.83 | 15.9 | 87.4 | 78.7 | High |
| 4 | Mouse | 1.33 | 4.47 | 310 | 1229 | High |
| | Rat | 2.76 | 11.8 | 117 | 211 | High |
| | Human | 23.7 | 28.3 | 49.0 | 44.1 | Medium |
| 7 | Mouse | 2.92 | 5.84 | 237 | 940 | High |
| | Rat | 2.06 | 2.30 | 603 | 1086 | High |
| | Human | 14.1 | 20.6 | 67.4 | 60.7 | High |
| 12 | Mouse | 26.9 | 33.6 | 41.2 | 163 | Medium |
| | Rat | 7.43 | 16.3 | 85.0 | 153 | High |
| | Human | 70.8 | 119 | 11.6 | 10.4 | Medium |
| 13 | Mouse | 38.6 | 44.7 | 31.0 | 123 | Medium |
| | Rat | 27.8 | 33.5 | 41.4 | 74.5 | Medium |
| | Human | 58.5 | 71.4 | 19.4 | 17.5 | Medium |
| 16 | Mouse | 24.6 | 29.9 | 46.4 | 184 | Medium |
| | Rat | 21.6 | 27.7 | 50.5 | 90.0 | Medium |
| | Human | 23.3 | 28.3 | 49.0 | 44.1 | Medium |
| 17 | Mouse | 2.46 | 11.7 | 119 | 470 | High |
| | Rat | 1.93 | 6.08 | 228 | 410 | High |
| | Human | 26.0 | 29.9 | 46.4 | 41.8 | Medium |
| 18 | Mouse | 4.32 | 13.4 | 103 | 409 | High |
| | Rat | 2.74 | 4.94 | 281 | 505 | High |
| | Human | 7.83 | 15.9 | 87.4 | 78.7 | High |
| 19 | Mouse | 22.0 | 28.5 | 48.6 | 192 | Medium |
| | Rat | 11.9 | 19.6 | 70.8 | 127 | Medium |
| | Human | 51.9 | 63.6 | 21.8 | 19.6 | Medium |
| 20 | Mouse | 15.9 | 23.1 | 60.0 | 238 | High |
| | Rat | 21.6 | 26.2 | 53.0 | 95.4 | Medium |
| | Human | 26.5 | 31.9 | 43.4 | 39.1 | Medium |
| 24 | Mouse | 58.2 | 73.7 | 18.8 | 74.4 | Medium |
| | Rat | 47.9 | 53.7 | 25.8 | 46.4 | Medium |
| | Human | 40.1 | 43.6 | 31.8 | 28.6 | Medium |

### Test Example 5. In Vivo Pharmacodynamic Study of Compound 1 in Mice

### 1. Test Materials:

Balb/C Nude mice, SPF grade, male, weighing 18-20 g, were purchased from Jiangsu Gempharmatech Co., Ltd.

HCT116 cells were purchased from ATCC.

Matrigel Matrix, Cat. No. 354248, was purchased from Becton, Dickinson and Company.

Kolliphor® HS 15, Cat. No. 42966, was purchased from Sigma-Aldrich.

### 2. Test Method:

a. Establishment of tumor-bearing mouse model: HCT116 cells in the logarithmic growth phase were taken and cultured, the cells were collected and counted, and then subcutaneously inoculated into Balb/C Nude mice at 1×106/mouse. The tumor volume and body weight of the mice were measured regularly. 7 days after inoculation, mice with tumor volume of 150 mm3 were randomly divided into groups and treated with corresponding drugs.

b. Animal grouping and administration: All mice were randomly divided into groups: 6 in the negative control group (sham group, 20% Solutol HS15), 6 in the onvansertib alone administration group (ONV-20 mg/kg group), and 6 in the compound 1 alone administration group (T1-20 mg/kg group). The administration was performed once a day by gavage for 21 consecutive days, and the tumor volume and body weight of the mice were measured regularly.

3. Data processing: All data were organized in EXCEL, and One-way ANOVA analysis and drawing were performed using GraphPad Prism 8.0 software. Statistical significance was achieved when p<0.05.

$$Tumor volume ( V ) = 0.5 \times Major axis \times Minor {axis}^{2}$$

$$Tumor inhibition rate ( TGI , \% ) = \frac{\begin{matrix}Average tumor volume in negative control group - \\ Average tumor volume in drug treatment group\end{matrix}}{Average tumor volume in negative control group} \times 100 \%$$

### 4. Test Results:

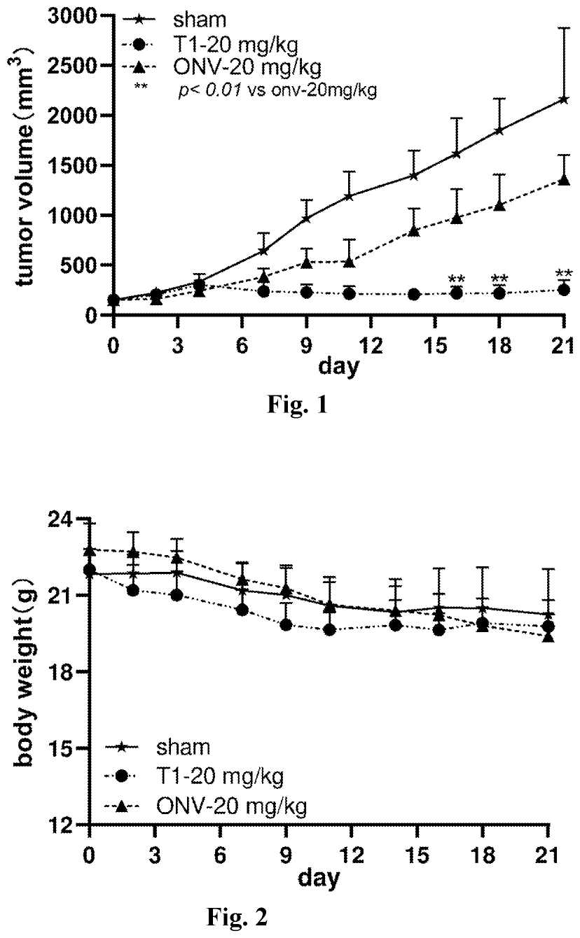

As shown in FIG. 1, when mice are treated with drugs for 11 days, compared to the sham group, the tumor inhibition rate of the T1-20 mg/kg group reaches 82.0% (p<0.0001), and the tumor inhibition rate in the ONV-20 mg/kg group is 54.9% (p<0.05). After continuous administration for 16 days, compared to the sham group, the tumor inhibition rate of the T1-20 mg/kg group reaches 86.5% (p<0.001), and the tumor inhibition rate in the ONV-20 mg/kg group is 39.6% (p<0.05), and there is a significant difference for the data of the T1-20 mg/kg and ONV-20 mg/kg group (p<0.01). After continuous administration for 21 days, compared to the sham group, the tumor inhibition rate of the T1-20 mg/kg group reaches 88.3% (p<0.0001), and the tumor inhibition rate in the ONV-20 mg/kg group is 37.0% (p<0.05), and there is a significant difference for the data of the T1-20 mg/kg and ONV-20 mg/kg group (p<0.01).

As shown in FIG. 2, during the test period, there is no significant difference in the body weight of animals in each group at each measurement.

5. Test conclusion: Compound 1 of the present invention has a significant inhibitory effect on the volume growth of colorectal tumors, and its efficacy at the same dose is better than that of the control drug onvansertib.

### Test Example 6. In Vivo Pharmacodynamic Study of Compound 18 in Mice

1. Experiment materials: Same as Test example 5.

### 2. Experimental Method:

a. Establishment of tumor-bearing mouse model: Same as Test example 5.

b. Animal grouping and administration: All mice were randomly divided into groups: 7 in the negative control group (control group, 20% Solutol HS15), 7 in the onvansertib alone administration group (onv-60 mg/kg group), and 7 in the compound 18 alone administration group (T18-10 mg/kg group). The administration was performed once a day by gavage for 7 consecutive days, and the tumor volume and body weight of the mice were measured regularly.

3. Data processing: Same as Test example 5.

### 4. Test Results:

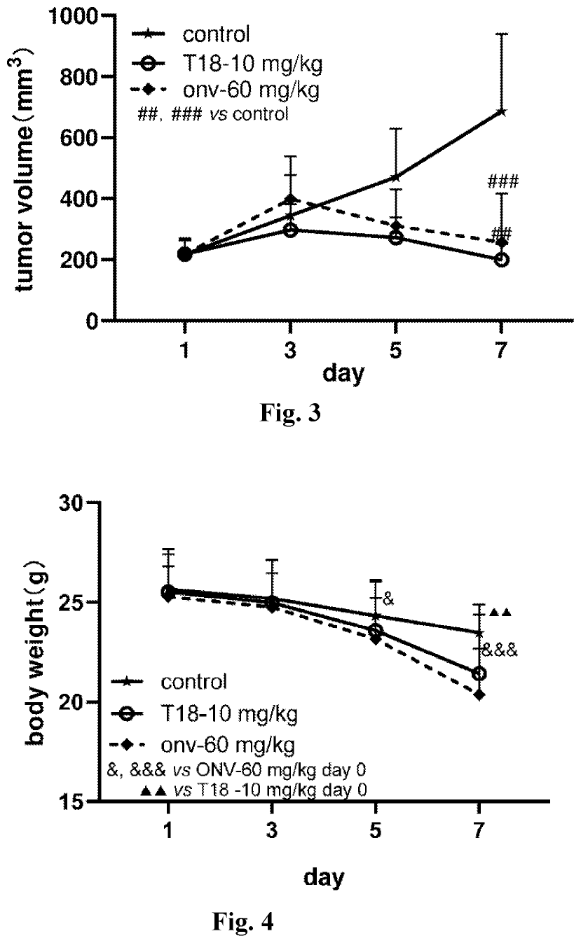

As shown in FIG. 3, when mice are treated with drugs for 7 days, compared to the control group, the T18-10 mg/kg group can significantly inhibit mouse tumor growth, with an inhibition rate of 70.9% (p<0.001), the tumor inhibition rate in the onv-60 mg/kg group is 62.7% (p<0.01).

As shown in FIG. 4, after continuous administration for 5 days, the body weight of the mice in the T18-10 mg/kg group decreased by 7.6% compared to that before the administration, and the body weight of the mice in the onv-60 mg/kg group decreased by 8.4% (p<0.05) compared to that before the administration. After continuous administration for 7 days, the body weight of the mice in the T18-10 mg/kg group decreased by 16.1% (p<0.01) compared to that before the administration, and the body weight of the mice in the onv-60 mg/kg group decreased by 19.5% (p<0.001) compared to that before the administration.

5. Test conclusion: Compound 18 of the present invention has a significant inhibitory effect on tumor volume growth in mice when administered continuously for 7 days at a dose of 10 mg/kg. To achieve the same efficacy, the required dose of compound 18 is approximately ⅙ of that of the control drug onvansertib, and its impact on animal body weight is less than that of the control drug.

1.-14. (canceled)

15. A compound of formula (I), or a pharmaceutically acceptable salt or stereoisomer thereof, 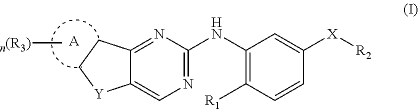
wherein X is independently selected from: divalent group of piperidine, piperazine, 3,8-diazabicyclo[3.2.1]octane, —CH₂-piperazine-, —NR-pyrrolidine-, —NR-azetidine-, or —NR—CH₂-pyrrolidine-; the R is independently selected from: H, D, or C₁-C₆ alkyl;
Y is independently selected from: divalent group of —CR₄═CR₄—, —CR₄═N— or —N═CR₄—, each of the R₄ is independently selected from: H, D, or C₁-C₆ alkyl;
ring A is selected from a structure represented by formula (A): 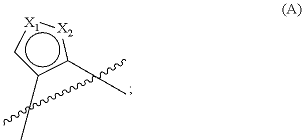
X₁ is independently selected from: C, N, O or S atom;
X₂ is independently selected from: C, N, O or S atoms;
R₁ is independently selected from: H, D, C₁-C₆ alkyl, C₁-C₆ haloalkyl, C₁-C₆ alkoxy, or C₁-C₆ haloalkoxy;
R₂ is independently selected from: H, D, or C₁-C₆ alkyl, wherein the C₁-C₆ alkyl is optionally substituted with one or more halogen, OH, NH₂, or CN groups;
each R₃ is independently selected from: H, D, C₁-C₆ alkyl, C₃-C₆ cycloalkyl, or —C(O)NHR₅, wherein the C₁-C₆ alkyl is optionally substituted with one or more halogen, OH, NH₂, CN, or C₁-C₆ alkoxy, the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, and the R₆ is independently selected from: C₁-C₆ alkyl, C₁-C₆ haloalkyl, or C₃-C₆ cycloalkyl.

16. The compound, or a pharmaceutically acceptable salt or stereoisomer thereof according to claim 15, wherein the compound is selected from: 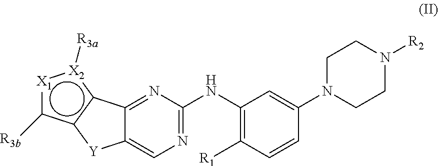
wherein:
Y is independently selected from: divalent group of —CR₄═CR₄—, —CR₄═N— or —N═CR₄—, each of the R₄ is independently selected from: H, D, or C₁-C₆ alkyl;
X₁ is independently selected from: C, N, O or S atoms;
X₂ is independently selected from: C, N, O or S atoms;
R₁ is independently selected from: H, D, C₁-C₆ alkyl, C₁-C₆ haloalkyl, C₁-C₆ alkoxy, or C₁-C₆ haloalkoxy;
R₂ is independently selected from: H, D, or C₁-C₆ alkyl, wherein the C₁-C₆ alkyl is optionally substituted with one or more halogen, OH, NH₂, or CN group;
R₃ₐ is independently selected from: H, D, C₁-C₆ alkyl, or C₃-C₆ cycloalkyl, wherein the C₁-C₆ alkyl is optionally substituted with one or more halogen, OH, NH₂, CN, or C₁-C₆ alkoxy;
R_{3b} is independently selected from: —C(O)NHR₅,
the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

17. The compound, or a pharmaceutically acceptable salt or stereoisomer thereof according to 16, wherein the compound is selected from: 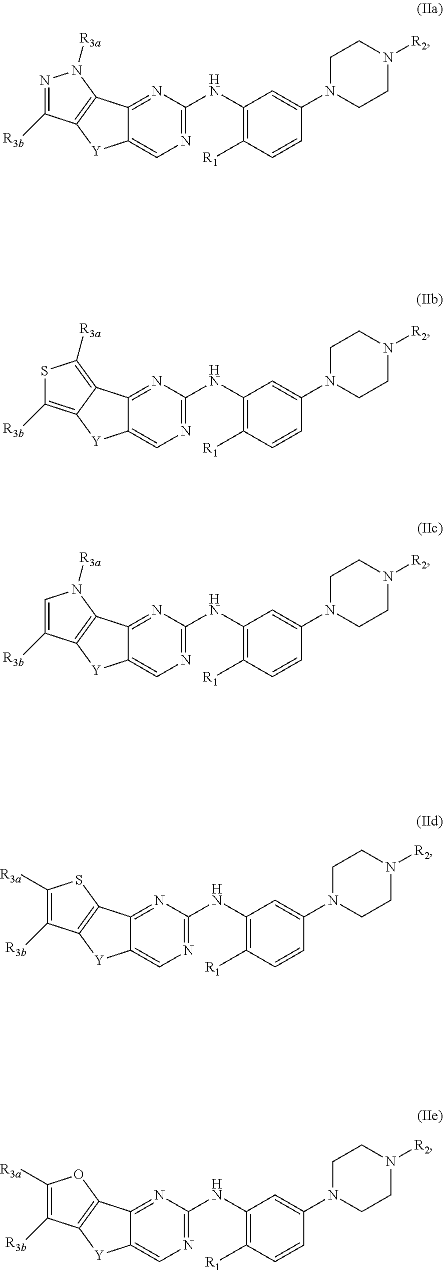
wherein:
Y is independently selected from: divalent group of —CR₄═CR₄—, —CR₄═N— or —N═CR₄—, each of the R₄ is independently selected from: H, D, or C₁-C₆ alkyl;
R₁ is independently selected from: H, D, C₁-C₆ alkyl, C₁-C₆ haloalkyl, C₁-C₆ alkoxy, or C₁-C₆ haloalkoxy;
R₂ is independently selected from: H, D, C₁-C₆ alkyl, wherein the C₁-C₆ alkyl is optionally substituted with one or more halogen, OH, NH₂, or CN group;
R₃ₐ is independently selected from: H, D, C₁-C₆ alkyl, C₃-C₆ cycloalkyl, wherein the C₁-C₆ alkyl is optionally substituted with one or more halogen, OH, NH₂, CN, or C₁-C₆ alkoxy,
R_{3b} is independently selected from: —C(O)NHR₅,
the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

18. The compound, or the pharmaceutically acceptable salt or stereoisomer thereof according to claim 15, wherein
X is preferably independently selected from: 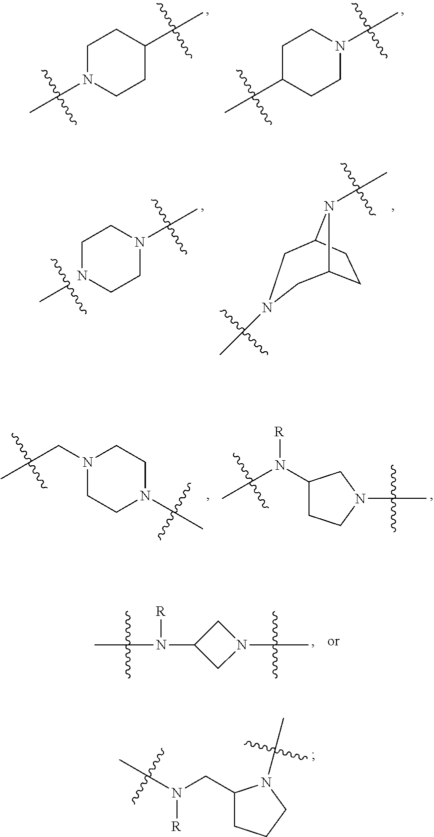
the R is independently selected from: H, D, methyl, ethyl, n-propyl, or isopropyl;
ring A is preferably selected from a structure represented by formula (A-1), formula (A-2), formula (A-3), formula (A-4) or formula (A-5): 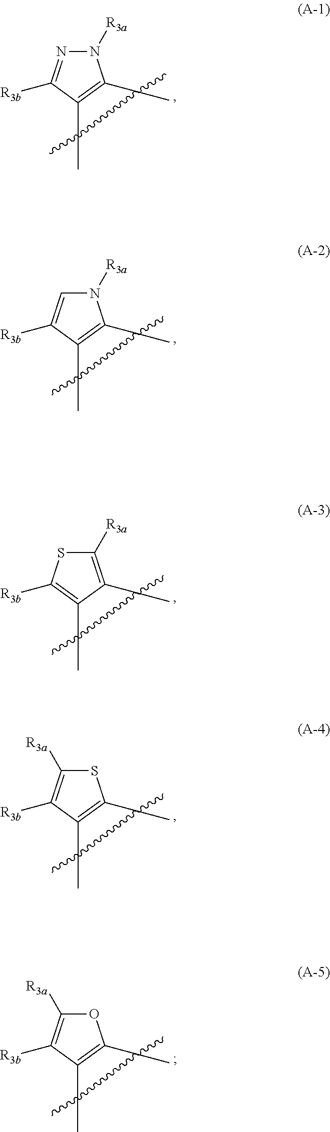
R₃ₐ is independently selected from: H, D, C₁-C₆ alkyl, C₃-C₆ cycloalkyl, wherein the C₁-C₆ alkyl is optionally substituted with one or more halogen, OH, NH₂, CN, or C₁-C₆ alkoxy;
R_{3b} is independently selected from: —C(O)NHR₅, the R₅ is independently selected from: H, D, C₁-C₆ alkyl, C₁-C₆ alkoxy, HO—C₁-C₆ alkoxy, OH, NH₂, —NHC(O)NH₂, or R₆—S(O)₂—, the R₆ is independently selected from: C₁-C₆ alkyl, C₁-C₆ haloalkyl, or C₃-C₆ cycloalkyl.

19. The compound, or the pharmaceutically acceptable salt or stereoisomer thereof according to claim 16, wherein, Y is preferably independently selected from: divalent group of —CH₂—CH₂—, —CH═CH—, —CH₂—NH—, —NH—CH₂—, —CH₂—N(CH₃)—, —N(CH₃)—CH₂—, —CH═N— or —N═CH—.

20. The compound or the pharmaceutically acceptable salt or stereoisomer thereof according to claim 16, wherein, R₁ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, methoxy, ethoxy, n-propoxy, isopropoxy, n-butoxy, sec-butoxy, tert-butoxy, n-pentyloxy, S-pentyloxy, hexyloxy, 2-ethylbutoxy, fluoromethoxy, chloromethoxy, difluoromethoxy, dichloromethoxy, trifluoromethoxy, trichloromethoxy, 2,2-difluoroethoxy, 2,2-dichloroethoxy, 3,3,3-trifluoroethoxy, 3,3,3-trichloroethoxy, pentafluoroethoxy, or pentachloroethoxy.

21. The compound or the pharmaceutically acceptable salt or stereoisomer thereof according to claim 16, wherein, R₂ is preferably independently selected from: H, D, methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl, wherein the terminal position of the methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, or 2-ethylbutyl is optionally substituted with one or more F, Cl, Br, OH, NH₂, or CN groups.

22. The compound or the pharmaceutically acceptable salt or stereoisomer thereof according to claim 16, wherein, R₃ₐ is preferably selected from: methyl, ethyl, n-propyl, n-butyl, n-pentyl, n-hexyl, cyclopropyl, cyclobutyl, cyclopentyl or cyclohexyl, wherein the terminal position of the methyl, ethyl, n-propyl, n-butyl, n-pentyl or n-hexyl is optionally substituted with one or more F, Cl, Br, OH, NH₂, CN, methoxy or ethoxy.

23. The compound or the pharmaceutically acceptable salt or stereoisomer thereof according to claim 16, wherein, R_{3b} is independently selected from: —C(O)NHR₅, the R₅ is particularly preferably selected from: H, D, methyl, ethyl, n-propyl, n-butyl, n-pentyl, n-hexyl, methoxy, ethoxy, n-propoxy, n-butoxy, n-pentyloxy, n-hexyloxy, HO-methoxy, HO-ethoxy, HO-n-propoxy, HO-n-butoxy, HO-n-pentyloxy, HO-n-hexyloxy, OH, NH₂, —NHC(O)NH₂, or R₆—S(O)₂—, and the R₆ is independently selected from: methyl, ethyl, n-propyl, isopropyl, n-butyl, isobutyl, s-butyl, t-butyl, n-pentyl, isopentyl, neopentyl, 1-ethylpropyl, n-hexyl, isohexyl, 1,1-dimethylbutyl, 2,2-dimethylbutyl, 3,3-dimethylbutyl, 2-ethylbutyl, fluoromethyl, chloromethyl, difluoromethyl, dichloromethyl, trifluoromethyl, trichloromethyl, 2,2-difluoroethyl, 2,2-dichloroethyl, 3,3,3-trifluoroethyl, 3,3,3-trichloroethyl, pentafluoroethyl, pentachloroethyl, cyclopropyl, cyclobutyl, cyclopentyl, or cyclohexyl.

24. A compound, or a pharmaceutically acceptable salt or stereoisomer thereof according to claim 16, selected from the following:

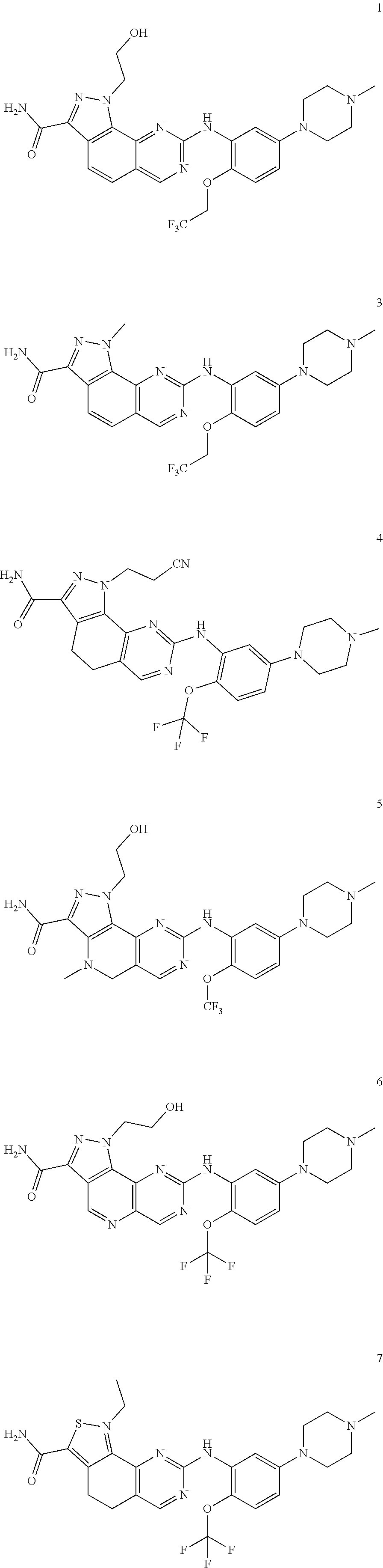

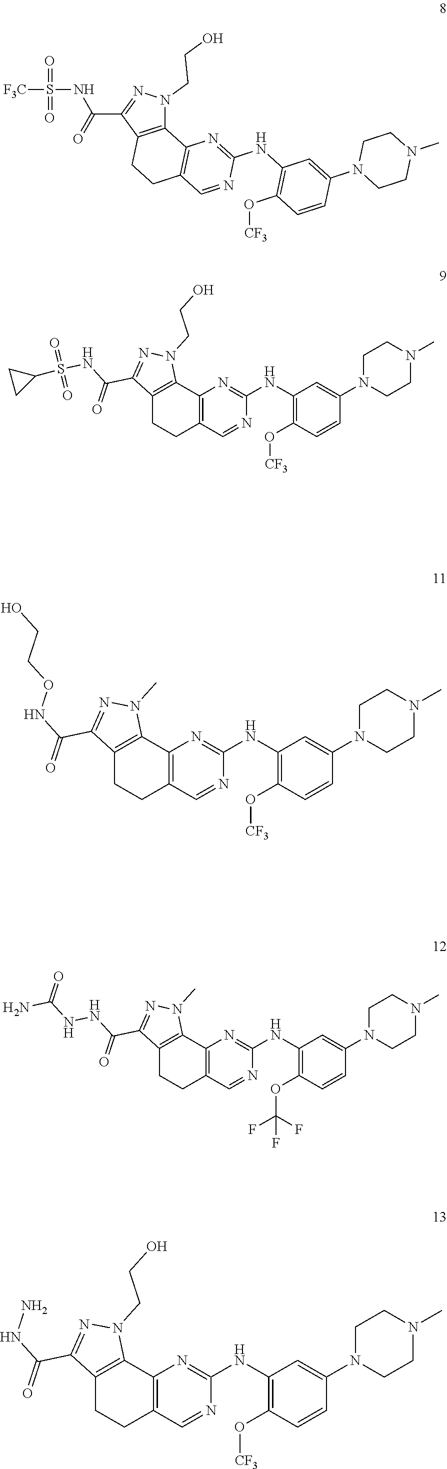 [SMILES: CN1CCN(c2ccc(OC(F)(F)F)c(Nc3ncc4c(n3)-c3c(c(C(=O)NS(=O)(=O)C(F)(F)F)nn3CCO)CC4)c2)CC1 ; CN1CCN(c2ccc(OC(F)(F)F)c(Nc3ncc4c(n3)-c3c(c(C(=O)NS(=O)(=O)C5CC5)nn3CCO)CC4)c2)CC1 ; CN1CCN(c2ccc(OC(F)(F)F)c(Nc3ncc4c(n3)-c3c(c(C(=O)NOCCO)nn3C)CC4)c2)CC1 ; CN1CCN(c2ccc(OC(F)(F)F)c(Nc3ncc4c(n3)-c3c(c(C(=O)NNC(N)=O)nn3C)CC4)c2)CC1 ; CN1CCN(c2ccc(OC(F)(F)F)c(Nc3ncc4c(n3)-c3c(c(C(=O)NN)nn3CCO)CC4)c2)CC1]

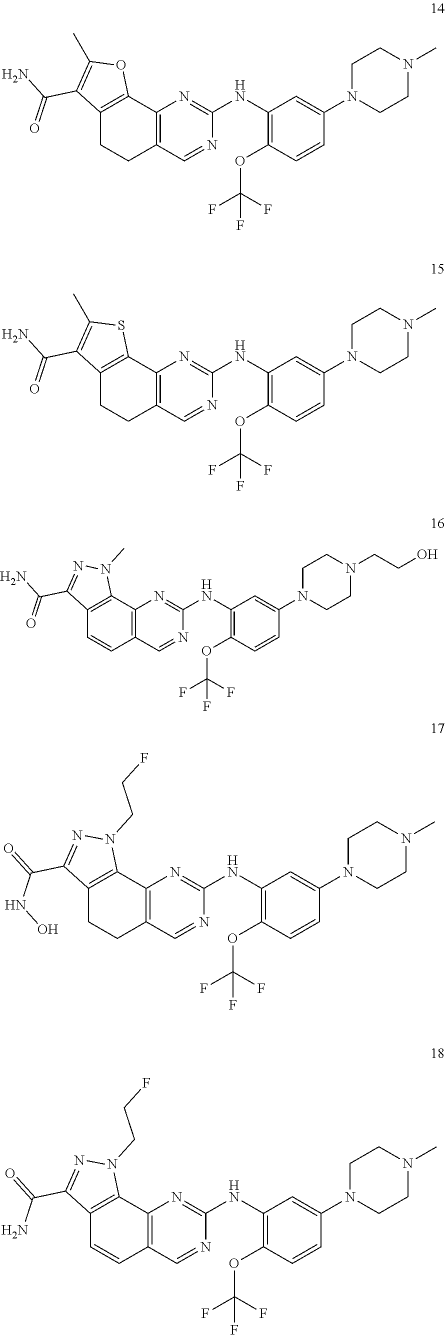 [SMILES: Cc1oc2c(c1C(N)=O)CCc1cnc(Nc3cc(N4CCN(C)CC4)ccc3OC(F)(F)F)nc1-2 ; Cc1sc2c(c1C(N)=O)CCc1cnc(Nc3cc(N4CCN(C)CC4)ccc3OC(F)(F)F)nc1-2 ; Cn1nc(C(N)=O)c2ccc3cnc(Nc4cc(N5CCN(CCO)CC5)ccc4OC(F)(F)F)nc3c21 ; CN1CCN(c2ccc(OC(F)(F)F)c(Nc3ncc4c(n3)-c3c(c(C(=O)NO)nn3CCF)CC4)c2)CC1 ; CN1CCN(c2ccc(OC(F)(F)F)c(Nc3ncc4ccc5c(C(N)=O)nn(CCF)c5c4n3)c2)CC1]

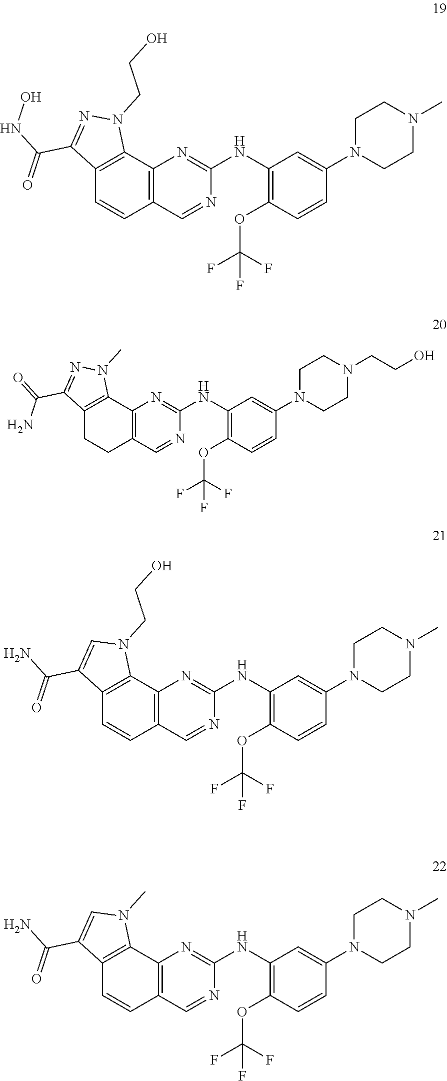 [SMILES: CN1CCN(c2ccc(OC(F)(F)F)c(Nc3ncc4ccc5c(C(=O)NO)nn(CCO)c5c4n3)c2)CC1 ; Cn1nc(C(N)=O)c2c1-c1nc(Nc3cc(N4CCN(CCO)CC4)ccc3OC(F)(F)F)ncc1CC2 ; CN1CCN(c2ccc(OC(F)(F)F)c(Nc3ncc4ccc5c(C(N)=O)cn(CCO)c5c4n3)c2)CC1 ; CN1CCN(c2ccc(OC(F)(F)F)c(Nc3ncc4ccc5c(C(N)=O)cn(C)c5c4n3)c2)CC1]

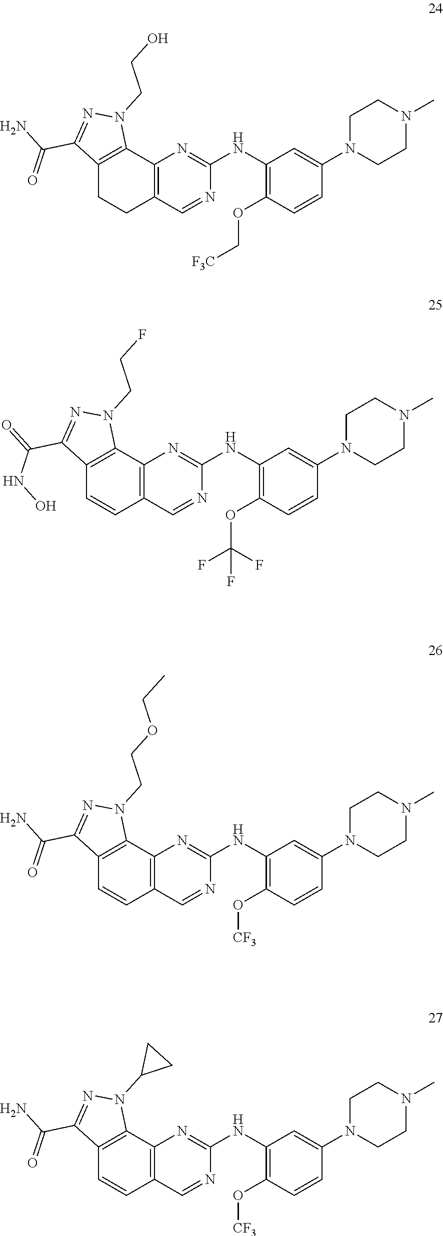 [SMILES: CN1CCN(c2ccc(OCC(F)(F)F)c(Nc3ncc4c(n3)-c3c(c(C(N)=O)nn3CCO)CC4)c2)CC1 ; CN1CCN(c2ccc(OC(F)(F)F)c(Nc3ncc4ccc5c(C(=O)NO)nn(CCF)c5c4n3)c2)CC1 ; CCOCCn1nc(C(N)=O)c2ccc3cnc(Nc4cc(N5CCN(C)CC5)ccc4OC(F)(F)F)nc3c21 ; CN1CCN(c2ccc(OC(F)(F)F)c(Nc3ncc4ccc5c(C(N)=O)nn(C6CC6)c5c4n3)c2)CC1]

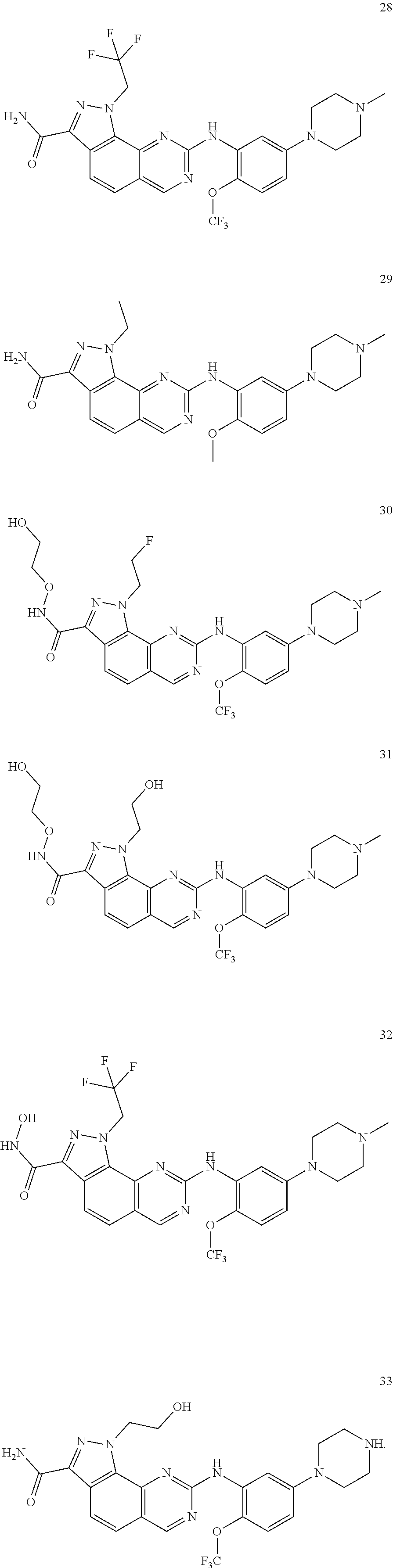 [SMILES: CN1CCN(c2ccc(OC(F)(F)F)c(Nc3ncc4ccc5c(C(N)=O)nn(CC(F)(F)F)c5c4n3)c2)CC1 ; CCn1nc(C(N)=O)c2ccc3cnc(Nc4cc(N5CCN(C)CC5)ccc4OC)nc3c21 ; CN1CCN(c2ccc(OC(F)(F)F)c(Nc3ncc4ccc5c(C(=O)NOCCO)nn(CCF)c5c4n3)c2)CC1 ; CN1CCN(c2ccc(OC(F)(F)F)c(Nc3ncc4ccc5c(C(=O)NOCCO)nn(CCO)c5c4n3)c2)CC1 ; CN1CCN(c2ccc(OC(F)(F)F)c(Nc3ncc4ccc5c(C(=O)NO)nn(CC(F)(F)F)c5c4n3)c2)CC1 ; NC(=O)c1nn(CCO)c2c1ccc1cnc(Nc3cc(N4CCNCC4)ccc3OC(F)(F)F)nc12]

25. A pharmaceutical composition, comprising the compound, or the pharmaceutically acceptable salt or stereoisomer thereof according to claim 15 and a pharmaceutically acceptable carrier.

26. A method for preventing or treating a disease caused by and/or related to dysregulation of protein kinase activity in a subject in need thereof, the method comprising administering to the subject a therapeutically effective amount of the compound, or the pharmaceutically acceptable salt or stereoisomer thereof according claim 15.

27. A method for preventing or treating a disease caused by and/or related to dysregulation of PLK1 kinase activity in a subject in need thereof, the method comprising administering to the subject a therapeutically effective amount of the compound, or the pharmaceutically acceptable salt or stereoisomer thereof according claim 15.

28. The method according to claim 27, wherein the disease is a cancer, a cell proliferative disease, a viral infection, an autoimmune disease and a neurodegenerative disease.
